(19) United States
(12) Patent Application Publication
(10) Pub. No.: US 2008/0267947 A1
(43) Pub. Date: Oct. 30, 2008

(54) Cancer therapy with cantharidin and cantharidin analogs

(76) Inventors: Thomas P. Cirrito, New York, NY (US); Ivan Bergstein, New York, NY (US)

Correspondence Address: JONES DAY, 222 EAST 41ST ST, NEW YORK, NY, 10017, US

(21) Appl. No.: 11/899,687

(22) Filed: Sep. 7, 2007

Related U.S. Application Data
(60) Provisional application No. 60/843,474, filed on Sep. 7, 2006.

(51) Int. Cl. A61K 39/395; A61K 31/351

(52) U.S. Cl. 424/130.1; 514/454

(57) ABSTRACT

Provided are methods for treating cancer in a patient, comprising administration of a therapeutically effective regimen of cantharidin or cantharidin analog of formula of formula I, II or III

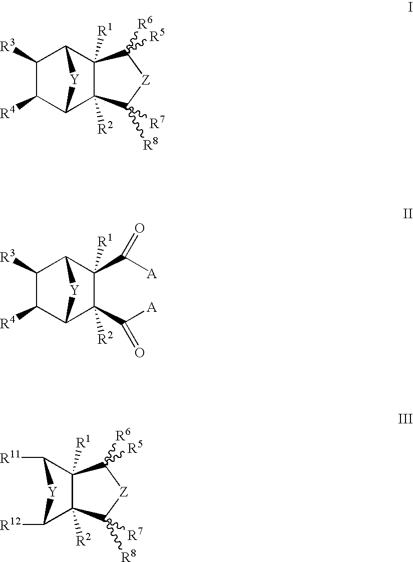

wherein R¹, R², R³, R⁴, R⁵, R⁶, R⁷, R⁸, R¹¹, R¹², A, Y and Z are as set forth herein, or a pharmaceutically acceptable salt thereof, to a patient in need thereof. In some embodiments of the methods, the therapeutically effective regimen stabilizes, reduces or eliminates cancer stem cells. In some embodiments of the methods, the therapeutically effective regimen reduces or eliminates cancer cells.

This application claims and is entitled to priority benefit of U.S. provisional application Ser. No. 60/843,474, filed Sep. 7, 2006, which is incorporated herein by reference in its entirety.

### 1. FIELD OF THE INVENTION

The present invention generally relates to methods for preventing, treating, and/or managing cancer, comprising administration of a prophylactically or therapeutically effective regimen of cantharidin, cantharidin analogs, and pharmaceutically acceptable salts thereof (e.g., norcantharidin and disodium cantharidate). In some embodiments of the methods, the prophylactically or therapeutically effective regimen stabilizes, reduces or eliminates cancer stem cells. In some embodiments of the methods, the therapeutically effective regimen stabilizes, reduces or eliminates cancer cells. The prophylactically or therapeutically effective regimen of the invention, in some embodiments, includes monitoring cancer stem cells in, or from, a patient receiving cantharidin, a cantharidin analog, or pharmaceutically acceptable salts thereof, and possibly altering the therapeutic regimen based on the results of such monitoring.

### 2. BACKGROUND OF THE INVENTION

2.1 Cancer Therapy

Cancer is one of the most significant health conditions. The American Cancer Society's Cancer Facts and Figures, 2003, predicts over 1.3 million Americans will receive a cancer diagnosis this year. In the United States, cancer is second only to heart disease in mortality accounting for one of four deaths. In 2002, the National Institutes of Health estimated total costs of cancer totaled $171.6 billion, with $61 billion in direct expenditures. The incidence of cancer is widely expected to increase as the US population ages, further augmenting the impact of this condition. The current treatment regimens for cancer, established in the 1970s and 1980s, have not changed dramatically. These treatments, which include chemotherapy, radiation and other modalities including newer targeted therapies, have shown limited overall survival benefit when utilized in most advanced stage common cancers since, among other things, these therapies primarily target tumor bulk rather than cancer stem cells.

More specifically, conventional cancer diagnosis and therapies to date have attempted to selectively detect and eradicate neoplastic cells that are largely fast-growing (i.e., cells that form the tumor bulk). Standard oncology regimens have often been largely designed to administer the highest dose of irradiation or a chemotherapeutic agent without undue toxicity, i.e., often referred to as the “maximum tolerated dose” (MTD) or “no observed adverse effect level” (NOAEL). Many conventional cancer chemotherapies (e.g., alkylating agents such as cyclophosphamide, antimetabolites such as 5-Fluorouracil, plant alkaloids such as vincristine) and conventional irradiation therapies exert their toxic effects on cancer cells largely by interfering with cellular mechanisms involved in cell growth and DNA replication. Chemotherapy protocols also often involve administration of a combination of chemotherapeutic agents in an attempt to increase the efficacy of treatment. Despite the availability of a large variety of chemotherapeutic agents, these therapies have many drawbacks (see, e.g., Stockdale, 1998, “Principles Of Cancer Patient Management” in Scientific American Medicine, vol. 3, Rubenstein and Federman, eds., ch. 12, sect. X). For example, chemotherapeutic agents are notoriously toxic due to non-specific side effects on fast-growing cells whether normal or malignant; e.g. chemotherapeutic agents cause significant, and often dangerous, side effects, including bone marrow depression, immunosuppression, gastrointestinal distress, etc.

Other types of traditional cancer therapies include surgery, hormonal therapy, immunotherapy, anti-angiogenesis therapy, targeted therapy (e.g. therapy directed to a cancer target such as Gleevec® and other tyrosine kinase inhibitors, Velcade®, Sutent®, et al.), and radiation treatment to eradicate neoplastic cells in a patient (see, e.g., Stockdale, 1998, “Principles of Cancer Patient Management,” in Scientific American: Medicine, vol. 3, Rubenstein and Federman, eds., ch. 12, sect. IV). All of these approaches can pose significant drawbacks for the patient including a lack of efficacy (in terms of long-term outcome (e.g. due to failure to target cancer stem cells) and toxicity (e.g. due to non-specific effects on normal tissues)). Accordingly, new therapies and/or regimens for improving the long-term prospect of cancer patients are needed.

2.2 Cancer Stem Cells

Cancer stem cells comprise a unique subpopulation (often 0.1-10% or so) of a tumor that, relative to the remaining 90% or so of the tumor (i.e., the tumor bulk), are more tumorigenic, relatively more slow-growing or quiescent, and often relatively more chemoresistant than the tumor bulk. Given that conventional therapies and regimens have, in large part, been designed to attack rapidly proliferating cells (i.e. those cancer cells that comprise the tumor bulk), cancer stem cells which are often slow-growing may be relatively more resistant than faster growing tumor bulk to conventional therapies and regimens. Cancer stem cells can express other features which make them relatively chemoresistant such as multi-drug resistance and anti-apoptotic pathways. The aforementioned would constitute a key reason for the failure of standard oncology treatment regimens to ensure long-term benefit in most patients with advanced stage cancers—i.e. the failure to adequately target and eradicate cancer stem cells. In some instances, a cancer stem cell(s) is the founder cell of a tumor (i.e., it is the progenitor of the cancer cells that comprise the tumor bulk).

Cancer stem cells have been identified in a large variety of cancer types. For instance, Bonnet et al., using flow cytometry were able to isolate the leukemia cells bearing the specific phenotype, CD34+CD38−, and subsequently demonstrate that it is these cells (comprising <1% of a given leukemia), unlike the remaining 99+% of the leukemia bulk, that are able to recapitulate the leukemia from whenst it was derived when transferred into immunodeficient mice. See, e.g., “Human acute myeloid leukemia is organized as a hierarchy that originates from a primitive hematopoietic cell,” Nat Med 3:730-737 (1997). That is, these cancer stem cells were found as <1 in 10,000 leukemia cells yet this low frequency population was able to initiate and serially transfer a human leukemia into severe combined immunodeficiency/non-obese diabetic (NOD/SCID) mice with the same histologic phenotype as in the original tumor.

Cox et al. identified small subfractions of human acute lymphoblastic leukemia (ALL) cells which had the phenotypes CD34⁺/CD 10⁻ and CD34⁺/CD19⁻, and were capable of engrafting ALL tumors in immunocompromised mice—i.e., the cancer stem cells. In contrast, no engraftment of the mice was observed using the ALL bulk, despite, in some cases, injecting 10-fold more cells. See Cox et al., “Characterization of acute lymphoblastic leukemia progenitor cells,” Blood 104(19): 2919-2925 (2004).

Multiple myeloma was found to contain small subpopulations of cells that were CD138⁻ and, relative to the large bulk population of CD138+myeloma cells had greater clonogenic and tumorigenic potential. See Matsui et al., “Characterization of clonogenic multiple myeloma cells,” Blood 103(6): 2332. The authors concluded that the CD138⁻ subpopulation of multiple myeloma was the cancer stem cell population.

Kondo et al. isolated a small population of cells from a C6-glioma cell line, which was identified as the cancer stem cell population by virtue of its ability to self-renew and recapitulate gliomas in immunocompromised mice. See Kondo et al., “Persistence of a small population of cancer stem-like cells in the C6 glioma cell line,” Proc. Natl. Acad. Sci. USA 101:781-786 (2004). In this study, Kondo et al. determined that cancer cell lines contain a population of cancer stem cells that confer the ability of the line to engraft immunodeficient mice.

Breast cancers were shown to contain a small population of cells with stem cell characteristics (bearing surface markers CD44+CD24^{low lin-}). See Al-Hajj et al., “Prospective identification of tumorigenic breast cancer cells,” Proc. Natl. Acad. Sci. USA 100:3983-3988 (2003). As few as 200 of these cells, corresponding to 1-10% of the total tumor cell population, are able to form tumors in NOD/SCID mice. In contrast, implantation of 20,000 cells that lacked this phenotype (i.e. the tumor bulk) was unable to re-grow the tumor.

A subpopulation of cells derived from human prostate tumors was found to self-renew and to recapitulate the phenotype of the prostate tumor from which they were derived thereby constituting the prostate cancer stem cell population. See Collins et al., “Prospective Identification of Tumorigenic Prostate Cancer Stem Cells,” Cancer Res 65(23): 10946-10951 (2005).

Fang et al. isolated a subpopulation of cells from melanoma with cancer stem cell properties. In particular, this subpopulation of cells could differentiate and self-renew. In culture, the subpopulation formed spheres whereas the more differentiated cell fraction from the lesions were more adherent. Moreover, the subpopulation containing sphere-like cells were more tumorigenic than the adherent cells when grafted into mice. See Fang et al., “A Tumorigenic Subpopulation with Stem Cell Properties in Melanomas,” Cancer Res 65(20): 9328-9337 (2005).

Singh et al. identified brain tumor stem cells. When isolated and transplanted into nude mice, the CD133+ cancer stem cells, unlike the CD133− tumor bulk cells, form tumors that can then be serially transplanted. See Singh et al., “Identification of human brain tumor initiating cells,” Nature 432:396-401 (2004); Singh et al., “Cancer stem cells in nervous system tumors,” Oncogene 23:7267-7273 (2004); Singh et al., “Identification of a cancer stem cell in human brain tumors,” Cancer Res. 63:5821-5828 (2003).

Since conventional cancer therapies target rapidly proliferating cells (i.e., cells that form the tumor bulk) these treatments are believed to be relatively ineffective at targeting and impairing cancer stem cells. In fact, cancer stem cells, including leukemia stem cells, have indeed been shown to be relatively resistant to conventional chemotherapeutic therapies (e.g. Ara-C, daunorubicin) as well as newer targeted therapies (e.g. Gleevec®, Velcade®). Examples of cancer stem cells from various tumors that are resistant to chemotherapy, and the mechanism by which they are resistant, are described in Table 1 below.

TABLE 1

| CSC Type | Resistance | Mechanism | Reference |
|---|---|---|---|
| AML | Ara-C | Quiescence | Guzman. Blood ′01 |
| AML | Daunorubicin | Drug Efflux, Anti- | Costello. Cancer |
| | | apoptosis | Res ′00 |
| AML | Daunorubicin, | Drug Efflux | Wulf. Blood ′01 |
| | mitoxantrone | | |
| AML | | Quiescence | Guan. Blood ′03 |
| AML, MDS | | Anti-apoptosis | Suarez. Clin Cancer |
| | | | Res ′04 |
| CML | | Quiescence | Holyoake. Blood ′99 |
| CML | Gleevec ® | Quiescence | Graham. Blood ′02 |
| Myeloma | Velcade ® | | Matsui. ASH 04 |

For example, leukemic stem cells are relatively slow-growing or quiescent, express multi-drug resistance genes, and utilize other anti-apoptotic mechanisms-features which contribute to their chemoresistance. See Jordan et al., “Targeting the most critical cells: approaching leukemia therapy as a problem in stem cell biology”, Nat Clin Pract Oncol. 2: 224-225 (2005). Further, cancer stem cells by virtue of their chemoresistance may contribute to treatment failure, and may also persist in a patient after clinical remission and these remaining cancer stem cells may therefore contribute to relapse at a later date. See Behbood et al., “Will cancer stem cells provide new therapeutic targets?” Carcinogenesis 26(4): 703-711 (2004). Therefore, targeting cancer stem cells is expected to provide for improved long-term outcomes for cancer patients. Accordingly, new therapeutic agents and/or regimens designed to target cancer stem cells are needed to reach this goal.

### 3. SUMMARY OF THE INVENTION

The invention provides a method of preventing, treating, and/or managing cancer in a patient, the method comprising administering to a patient in need thereof a prophylactically effective regimen or a therapeutically effective regimen, the regimen comprising administering cantharidin or a cantharidin analog of formula I, II or III.

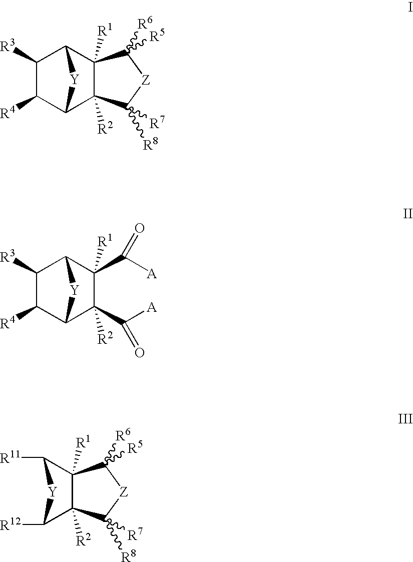

wherein

R¹ and R² are independently H or CH₃;

R³ and R⁴ are independently H, C₁-C₆ alkyl, aryl, or ara (C₁-C₁₀)alkyl; or together R³ and R⁴ form a bond (i.e., to form a cyclohexenyl ring);

R¹, R⁶, R⁷, and R⁸ are independently H or OH, or R⁵ and R⁶, or R⁷ and R⁸ together with the carbon to which they are attached, form C═O;

R¹¹ and R¹² are independently H, C₁-C₁₀ alkyl, aryl, or ara (C₁-C₁₀)alkyl;

Y is O, N, or S;

A is OH or OR¹⁰, wherein R¹⁰ is C₁-C₆ alkyl;

Z is O, S, SR¹⁴, N—R⁹, CH₂OR¹², CHQ, or an amino acid;

R¹⁴ is C₁-C₁₀ alkyl, aryl, or ara(C₁-C₁₀)alkyl;

R⁹ is C₁-C₁₀ alkyl, H, OH, or Q;

R¹² is H or C₁-C₁₀ alkyl, aryl, or ara(C₁-C₁₀)alkyl;

wherein when Z is an amino acid, the α-amino group is a ring atom in a five-membered ring of formula I or III;

wherein Q is H or a moiety having the formula

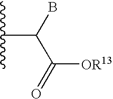

wherein R¹³ is C₁-C₁₀ alkyl or H; and B is (CH₂)ₙW, CH═CH—W, or CH₂OW, wherein W is an ionisable residue; or

a pharmaceutically acceptable salt thereof, to the patient.

The invention also provides compounds represented by formula II, wherein A is OH or NR¹¹R¹².

R¹¹ or R¹² are independently H, substituted or unsubstituted C₁₋₈cycloalkyl, R¹¹R¹²N—C₁₋₈alkyl, NR¹¹R¹², substituted or unsubstituted aryl-C₁₋₈-alkyl, or substituted or unsubstituted heteroaryl-C₁₋₈alkyl. Furthermore the R¹¹ or R¹² are independently H or substituted or unsubstituted aryl.
R¹¹ and R¹² together with the nitrogen to which they are attached may form a substituted or unsubstituted saturated heterocycle, wherein one CH₂ group in the saturated heterocycle may be replaced by O, NR¹⁰, S, S(═O) or S(═O)₂

In certain embodiments compounds of the invention are represented by formula I, wherein:

### Z is NR¹³;

R¹³ is a substituted or unsubstituted aryl or substituted or unsubstituted heteroaryl; and
R¹, R², R³, R⁴, R⁵, R⁶, R⁷, R⁸ and Y are as defined above.

In an additional embodiment, compounds of the invention are represented by formula I, wherein:

### Z is NR¹³;

R¹³ is a substituted or unsubstituted 8 to 10 membered unsaturated heterobicyclic system with one or more heteroatoms independently selected from N, O and S; and
R¹, R², R³, R⁴, R⁵, R⁶, R⁷, R⁸ and Y are as defined above.

R¹ and R² are independently H or CH₃;

R³ and R⁴ are independently H, C₁-C₆ alkyl, or aryl; or together R³ and R⁴ form a bond (i.e., to form a cyclohexenyl ring);

R⁵, R⁶, R⁷, and R⁸ are independently H or OH, or R⁵ and R⁶, or R⁷ and R⁸ together with the carbon to which they are attached, form C═O;

Y is O, N, or S;

Z is PtL₂, wherein each L is ammonia or together form a bidentate bis amino ligand such as a substituted or unsubstituted cyclohexane-1,2-diamine; or

a pharmaceutically acceptable salt thereof.

In one aspect, the invention provides a method of preventing, treating, and/or managing cancer in a patient in need thereof, the method comprising administering a prophylactically effective regimen or a therapeutically effective regimen, the regimen comprising administering to the patient a compound of formula I, II or III (as described above), or a pharmaceutically acceptable salt thereof, wherein the patient has been diagnosed with cancer, and wherein said cancer is a hematologic cancer. In some embodiments, the patient received a therapy for the treatment and/or management of the cancer before the administration of the therapeutically effective regimen of the compound of formula I, II or III. Non-limiting examples of such a therapy include chemotherapy, radioimmunotherapy, hormonal therapy, small molecule therapy, toxin therapy, prodrug-activating enzyme therapy, biologic therapy, antibody therapy, surgical therapy, including immunotherapy, anti-angiogenic therapy, targeted therapy, epigenetic therapy, demethylation therapy, histone deacetylase inhibitor therapy, differentiation therapy, radiation therapy, and/or any combination thereof. In some embodiments, the patient has not previously received a therapy for the treatment and/or management of the cancer. In a specific embodiment, the hematologic cancer is leukemia, lymphoma, myeloma or myelodysplastic syndrome.

In another aspect, the invention provides a method of preventing, treating, and/or managing a solid tumor in a patient, the method comprising administering to a patient in need thereof a prophylactically effective regimen or a therapeutically effective regimen, the regimen comprising administering to the patient a compound of formula I, II or III (as described above), or a pharmaceutically acceptable salt thereof wherein the patient has been diagnosed with a solid tumor, and wherein the patient has undergone primary therapy to reduce the bulk of the tumor. In some embodiments, the primary therapy is, for example, chemotherapy, radioimmunotherapy, hormonal therapy, small molecule therapy, biologic therapy, toxin therapy, prodrug-activating enzyme therapy, antibody therapy, surgical therapy, immunotherapy, anti-angiogenic therapy, targeted therapy, differentiation therapy, epigenetic therapy, demethylation therapy, histone deacetylase inhibitor therapy, radiation therapy, or any combination thereof.

In particular embodiments of this aspect, the solid tumor is fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, melanoma, neuroblastoma, or retinoblastoma.

In another aspect, the invention provides a method of preventing, treating, and/or managing cancer, the method comprising administering to a patient in need thereof a prophylactically effective regimen or a therapeutically effective regimen, the regimen comprising administering to the patient a compound of formula I, II or III (as described above), or a pharmaceutically acceptable salt thereof wherein the patient received another therapy. In some embodiments, the prior therapy is, for example, chemotherapy, radioimmunotherapy, hormonal therapy, small molecule therapy, toxin therapy, prodrug-activating enzyme therapy, antibody therapy, surgical therapy, biologic therapy including immunotherapy, anti-angiogenic therapy, targeted therapy, epigenetic therapy, demethylation therapy, histone deacetylase inhibitor therapy, protein therapy, differentiation therapy, radiation therapy or any combination thereof.

In another aspect, the invention provides a method of treating cancer in a patient, the method comprising administering to a patient in need thereof a prophylactically effective regimen, the regimen comprising administering to the patient a compound of formula I, II or III (as described above), or a pharmaceutically acceptable salt thereof, wherein the patient is in remission for the cancer.

In a specific aspect, the invention provides a method of preventing, treating, and/or managing cancer in a patient, the method comprising administering to a patient in need thereof, a prophylactically effective regimen or a therapeutically effective regimen, the regimen comprising administering to the patient a compound of formula I, II or III (as described above), or a pharmaceutically acceptable salt thereof, wherein the compound of formula I, II or III is administered at a dose lower than the maximum tolerated dose (MTD) over a period of 1 to 3 months, 3 to 6 months, 1 to 12 months, or 6 to 12 months. In another embodiment the compound of formula I, II, or III is administered at a dose lower than the MTD over a longer period of time such as 9, 12, 24, 36, 48 months, or for the remainder of the patient's life. In one embodiment, the dose of the compound of formula I, II or III administered to the patient is from 0.1 to 50 mg/m².

In a specific aspect, the invention provides a method of preventing, treating, and/or managing cancer in a patient, the method comprising administering to a patient in need thereof, a prophylactically effective regimen or a therapeutically effective regimen, the regimen comprising administering to the patient a compound of formula I, II or III (as described above), or a pharmaceutically acceptable salt thereof, wherein the compound of formula I, II or III is administered at a dose lower than the human equivalent dose (HED) of the no observed adverse effect level (NOAEL) over a period of 1 to 3 months, 3 to 6 months, 1 to 12 months, or 6 to 12 months. In another embodiment, the compound of formula I, II, or III is administered at a dose lower than the HED of the NOAEL over a longer period of time, such as 9, 12, 24, 36, 48 months, or for the remainder of the patient's life. In one embodiment, the dose of the compound of formula I, II or III administered to the patient is from 0.1 to 50 mg/m².

In a specific aspect, the invention provides a method of preventing, treating, and/or managing kidney cancer in a patient, the method comprising administering to a patient in need thereof, a prophylactically effective regimen or a therapeutically effective regimen, the regimen comprising administering to the patient a compound of formula I, II or III (as described above), or a pharmaceutically acceptable salt thereof, wherein the patient has been diagnosed with kidney cancer.

In a specific aspect, the invention provides a method of preventing, treating, and/or managing pancreatic cancer in a patient, the method comprising administering to a patient in need thereof, a prophylactically effective regimen or a therapeutically effective regimen, the regimen comprising administering to the patient a compound of formula I, II or III (as described above), or a pharmaceutically acceptable salt thereof, wherein the patient has been diagnosed with pancreatic cancer.

In a specific aspect, the invention provides a method of preventing, treating, and/or managing bone cancer in a patient, the method comprising administering to a patient in need thereof, a prophylactically effective regimen or a therapeutically effective regimen, the regimen comprising administering to the patient a compound of formula I, II or III (as described above), or a pharmaceutically acceptable salt thereof, wherein the patient has been diagnosed with bone cancer.

In a specific aspect, the invention provides a method of preventing, treating, and/or managing breast cancer in a patient, the method comprising administering to a patient in need thereof, a prophylactically effective regimen or a therapeutically effective regimen, the regimen comprising administering to the patient a compound of formula I, II or III (as described above), or a pharmaceutically acceptable salt thereof, wherein the patient has been diagnosed with breast cancer.

In a specific aspect, the invention provides a method of preventing, treating, and/or managing ovarian cancer in a patient, the method comprising administering to a patient in need thereof, a prophylactically effective regimen or a therapeutically effective regimen, the regimen comprising administering to the patient a compound of formula I, II or III (as described above), or a pharmaceutically acceptable salt thereof, wherein the patient has been diagnosed with ovarian cancer.

In a specific aspect, the invention provides a method of preventing, treating, and/or managing cervical cancer in a patient, the method comprising administering to a patient in need thereof, a prophylactically effective regimen or a therapeutically effective regimen, the regimen comprising administering to the patient a compound of formula I, II or III (as described above), or a pharmaceutically acceptable salt thereof, wherein the patient has been diagnosed with cervical cancer.

In a specific aspect, the invention provides a method of preventing, treating, and/or managing uterine cancer in a patient, the method comprising administering to a patient in need thereof, a prophylactically effective regimen or a therapeutically effective regimen, the regimen comprising administering to the patient a compound of formula I, II or III (as described above), or a pharmaceutically acceptable salt thereof, wherein the patient has been diagnosed with uterine cancer.

In a specific aspect, the invention provides a method of preventing, treating, and/or managing testicular cancer in a patient, the method comprising administering to a patient in need thereof, a prophylactically effective regimen or a therapeutically effective regimen, the regimen comprising administering to the patient a compound of formula I, II or III (as described above), or a pharmaceutically acceptable salt thereof, wherein the patient has been diagnosed with testicular cancer.

In a specific aspect, the invention provides a method of preventing, treating, and/or managing bladder cancer in a patient, the method comprising administering to a patient in need thereof, a prophylactically effective regimen or a therapeutically effective regimen, the regimen comprising administering to the patient a compound of formula I, II or III (as described above), or a pharmaceutically acceptable salt thereof, wherein the patient has been diagnosed with bladder cancer.

In a specific aspect, the invention provides a method of preventing, treating, and/or managing skin cancer in a patient, the method comprising administering to a patient in need thereof, a prophylactically effective regimen or a therapeutically effective regimen, the regimen comprising administering to the patient a compound of formula I, II or III (as described above), or a pharmaceutically acceptable salt thereof, wherein the patient has been diagnosed with skin cancer.

In a specific aspect, the invention provides a method of preventing, treating, and/or managing melanoma in a patient, the method comprising administering to a patient in need thereof, a prophylactically effective regimen or a therapeutically effective regimen, the regimen comprising administering to the patient a compound of formula I, II or III (as described above), or a pharmaceutically acceptable salt thereof, wherein the patient has been diagnosed with melanoma.

In a specific aspect, the invention provides a method of preventing, treating, and/or managing neuroblastoma in a patient, the method comprising administering to a patient in need thereof, a prophylactically effective regimen or a therapeutically effective regimen, the regimen comprising administering to the patient a compound of formula I, II or III (as described above), or a pharmaceutically acceptable salt thereof, wherein the patient has been diagnosed with neuroblastoma.

In a specific aspect, the invention provides a method of preventing, treating, and/or managing retinoblastoma in a patient, the method comprising administering to a patient in need thereof, a prophylactically effective regimen or a therapeutically effective regimen, the regimen comprising administering to the patient a compound of formula I, II or III (as described above), or a pharmaceutically acceptable salt thereof, wherein the patient has been diagnosed with retinoblastoma. In some embodiments of the above-described aspects, the regimens comprise the administration of the compound of formula I, II or III over a period of 1 to 3 months, 3 to 6 months, 1 to 12 months, or 6 to 12 months. In some other embodiments the regimens comprise the administration of the compound of formula I, II, or III over a longer period of time such as 9, 12, 24, 36, 48 months or for the remainder of the patient's life. In some embodiments of the above-described aspects, the regimens result in a reduction in the cancer cell population. In a specific embodiment, the regimens result in a 5% to 40%, preferably a 10% to 60%, and more preferably, a 20% to 98% reduction in the cancer cell population.

In certain embodiments of the above-described aspects, the regimens further comprise monitoring the cancer cell population in the patient. In specific embodiments, the monitoring comprises detecting in a specimen from said patient the amount of cancer cells in said specimen. In some embodiments, the specimen is a blood specimen, bone marrow sample, a tissue biopsy, or a tumor biopsy. The regimens, in specific embodiments, comprise administering a second effective amount of the compound of formula I, II or III to the patient.

In some embodiments of the above-described aspects, the regimens result in a reduction in the cancer stem cell population. In a specific embodiment, the regimens result in a 5% to 40%, preferably a 10% to 60%, and more preferably, a 20% to 98% reduction in the cancer stem cell population.

In certain embodiments of the above-described aspects, the regimens further comprise monitoring the cancer stem cell population in the patient. In specific embodiments, the monitoring comprises detecting in a specimen from said patient the amount of cancer stem cells in said specimen. In some embodiments, the specimen is a blood specimen, bone marrow sample, a tissue biopsy, or a tumor biopsy. The regimens, in specific embodiments, comprise administering a second effective amount of the compound of formula I, II or III to the patient. In some embodiments of the above-described aspects, the regimens comprise intravenous or subcutaneous administration of the compound of formula I, II or III. In one embodiment, the regimen comprises intravenous administration of the compound of formula I, II or III in a dose of 50 mg/kg or less. In another embodiment, the regimens comprise subcutaneous administration of the compound of formula I, II or III in a dose of 50 mg/kg or less.

In certain embodiments of the above-described aspects, the regimens further comprise the administration of an additional therapy to the patient, wherein the compound of formula I, II or III and the additional therapy are administered separately, concurrently, or sequentially. The additional therapy can be, for example, chemotherapy, radioimmunotherapy, hormonal therapy, small molecule therapy, biologic therapy, toxin therapy, prodrug-activating enzyme therapy, surgical therapy, immunotherapy, anti-angiogenic therapy, radiation therapy, epigenetic therapy, demethylation therapy, histone deacetylase inhibitor therapy, differentiation therapy, targeted therapy or any combination thereof. In a specific embodiment, the additional therapy is chemotherapy, wherein the regimen comprises the administration of the compound of formula I, II or III in combination with an additional therapy. The compound of formula I, II or III and the additional therapy can be administered separately, concurrently, or sequentially. In certain embodiments of the above-described aspects, the regimens comprise administration of a compound of formula I or II

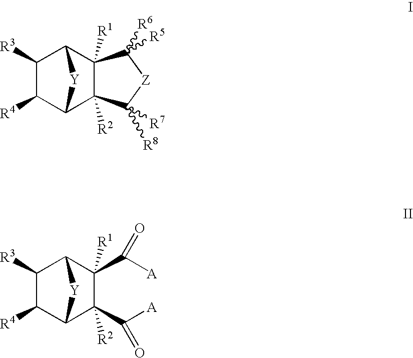

wherein

R¹ and R² are independently, H or CH₃;

R³ and R⁴ are independently, H, C₁-C₆ alkyl, or aryl; or together R³ and R⁴ form a bond;

R⁵, R⁶, R⁷, and R⁸ are independently H or OH, or R⁵ and R⁶, or R⁷ and R⁸ together with the carbon to which they are attached, form C═O;

Y is O, N, or S;

Z is O, S, NOH, or N—R⁹, wherein R⁹ is C₁-C₆ alkyl or H; and

A is OH or OR¹⁰, wherein R¹⁰ is C₁-C₆ alkyl; or

a pharmaceutically acceptable salt thereof, to the patient.

In certain embodiments of the above-described aspects, the regimens comprise administration of a compound of formula I to the patient, wherein

R¹ and R² are both CH₃, R³ and R⁴ are both H,

R⁵ and R⁶ together with the carbon to which they are attached form C═O,

R⁷ and R⁸ together with the carbon to which they are attached form C═O, and

Y and Z are both O.

In such embodiments, the compound is typically administered to the patient at a dose ranging from 0.1 to 25 mg/kg or 25 to 50 mg/kg.

In other embodiments of the above-described aspects, the regimens comprise administration of a compound of formula I to the patient, wherein
R¹, R², R³, and R⁴ are H,
R⁵ and R⁶ together with the carbon to which they are attached form C═O,
R⁷ and R⁸ together with the carbon to which they are attached form C═O, and Y and Z are both O.

In such embodiments, the compound is typically administered to the patient at a dose ranging from 0.1 to 25 mg/kg or 25 to 50 mg/kg.

In other embodiments of the above-described aspects, the regimens comprise administration of a compound of formula II to the patient, wherein

R¹ and R² are both CH₃, R³ and R⁴ are both H,

Y is O, and

A is OH;

or a pharmaceutically acceptable salt thereof.
In specific embodiments, the compound of the formula II is disodium cantharidate. In such embodiments, the compound is typically administered to the patient at a dose ranging from 0.1 to 25 mg/kg or 25 to 50 mg/kg.

### 3.1 DEFINITIONS

As used herein, the term “agent” refers to any molecule, compound, and/or substance for use in the prevention, treatment, management and/or diagnosis of cancer.

As used herein, the terms “about” or “approximately”, unless otherwise indicated, refer to a value that is no more than 10% above or below the value being modified by the term.

As used herein, the term “significantly,” as used in the context of purging of the bone marrow or peripheral blood of cancer stem cells refers to a decrease in cancer stem cells by least 50%, 60%, 75%, 80%, 90%, 95%, or by at least 99%.

As used herein, the term “refractory” is most often determined by failure to reach clinical endpoint, e.g., response, extended duration of response, extended disease free survival, relapse free survival, progression free survival, and overall survival. Another way to define being refractory to a therapy is that a patient has failed to achieve a response to a therapy such that the therapy is determined to not be therapeutically effective.

As used herein, the term, “alkenyl” means a linear or branched aliphatic hydrocarbon group containing a carbon-carbon double bond having a single radical and 2-10 carbon atoms. A “branched” alkenyl means that one or more alkyl groups such as methyl, ethyl or propyl replace one or both hydrogens in a —CH₂— or —CH═ linear alkenyl chain. Exemplary alkenyl groups include ethenyl, 1- and 2-propenyl, 1-, 2- and 3-butenyl, 3-methylbut-2-enyl, 2-propenyl, heptenyl, octenyl and decenyl.

As used herein, the term “alkyl” means a linear or branched saturated aliphatic hydrocarbon group having a single radical and 1-10 carbon atoms. Examples of alkyl groups include methyl, propyl, isopropyl, butyl, n-butyl, isobutyl, sec-butyl, tert-butyl, and pentyl. A branched alkyl means that one or more alkyl groups such as methyl, ethyl or propyl, replace one or both hydrogens in a —CH₂— group of a linear alkyl chain. The term “lower alkyl” means an alkyl of 1-3 carbon atoms.

As used herein, the term “antibodies” refer to molecules that contain an antigen binding site, e.g., immunoglobulins. Immunoglobulin molecules can be of any type (e.g., IgG, IgE, IgM, IgD, IgA and IgY), class (e.g., IgG1, IgG2, IgG3, IgG4, IgA1 and IgA2) or subclass. Antibodies include, but are not limited to, monoclonal antibodies, multispecific antibodies, human antibodies, humanized antibodies, camelized antibodies, chimeric antibodies, single domain antibodies, single chain Fvs (scFv), single chain antibodies, Fab fragments, F(ab′) fragments, disulfide-linked Fvs (sdFv), and anti-idiotopic (anti-Id) antibodies (including, e.g., anti-Id antibodies to antibodies of the invention), and epitope-binding fragments of any of the above.

As used herein, the terms “antibody conjugate(s)” and “antibody fragment conjugate(s)” refer to a conjugate(s) of an antibody or antibody fragment that is prepared by way of a synthetic chemical reaction(s) or as a recombinant fusion protein(s).

As used herein, the term “aryl” means a carbocyclic aromatic ring system containing one, two or three rings which may be attached together in a pendent manner or fused, and containing a single radical. Exemplary aryl groups include phenyl and naphthyl.

A “heteroaryl” group is an aryl ring system having one to four heteroatoms as ring atoms in a heteroaromatic ring system, wherein the remainder of the atoms are carbon atoms. Suitable heteroatoms include oxygen, sulfur and nitrogen. In certain embodiments, the heteroaryl ring system is monocyclic or bicyclic. Non-limiting examples include the following:

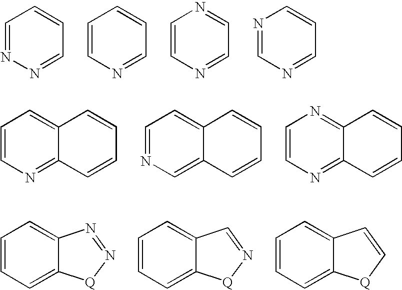

wherein Q is CH2, C═CH2, O, S or NH. A heteroaryl group can be substituted or unsubstituted. Representative examples of a heteroaryl group include, but are not limited to, furyl, benzofuranyl, benzothiazolyl, benzothienyl, indolyl, benzopyrazolyl, coumarinyl, pyrimidinyl, isoquinolinyl, quinolinyl, pyridinyl and pyrazinyl. In one embodiment, the heteroaryl group is a C3-10heteroaryl group.

As used herein, the term “cancer” refers to a neoplasm or tumor resulting from abnormal uncontrolled growth of cells. Non-limiting examples include those cancers described in Section 5.3.3, infra. The term “cancer” encompasses a disease involving both pre-malignant and malignant cancer cells. In some embodiments, cancer refers to a localized overgrowth of cells that has not spread to other parts of a subject, i.e., a benign tumor. In other embodiments, cancer refers to a malignant tumor, which has invaded and destroyed neighboring body structures and spread to distant sites. In yet other embodiments, the cancer is associated with a specific cancer antigen.

As used herein, the term “administer continuously,” in the context of administration of a therapy to a subject, refers to the administration of a therapy to a subject at a frequency that is expected to maintain a specific plasma concentration of the therapy. For instance, in some embodiments of the therapies that are administered continuously, the administration to the subject is at a frequency that is expected to maintain less than a 50% change in the plasma concentration of the therapy, e.g., a 20-50% change, a 10-30% change, a 5-25% change, or a 1-20% change in plasma concentration of the therapy.

As used herein, the term “amount,” as used in the context of the amount of a particular cell population or cells, refers to the frequency, quantity, percentage, relative amount, or number of the particular cell population or cells.

As used herein, the term “cancer cells” refer to cells that acquire a characteristic set of functional capabilities during their development, including the ability to evade apoptosis, self-sufficiency in growth signals, insensitivity to anti-growth signals, tissue invasion/metastasis, significant growth potential, and/or sustained angiogenesis. The term “cancer cell” is meant to encompass both pre-malignant and malignant cancer cells.

As used herein, the term “cancer stem cell(s)” refers to a cell that can be a progenitor of a highly proliferative cancer cell. A cancer stem cell has the ability to re-grow a tumor as demonstrated by its ability to form tumors in immunocompromised mice, and typically to form tumors upon subsequent serial transplantation in immunocompromised mice. Cancer stem cells are also typically slow-growing relative to the bulk of a tumor; that is, cancer stem cells are generally quiescent. In certain embodiments, but not all, the cancer stem cell may represent approximately 0.1 to 10% of a tumor.

As used herein, the term “chiral center” refers to a carbon atom to which four different groups are attached.

As used herein, the term “cycloalkenyl” means a non-aromatic monocyclic or multicyclic hydrocarbon ring system containing a carbon-carbon double bond having a single radical and 3 to 12 carbon atoms. Exemplary monocyclic cycloalkenyl rings include cyclopropenyl, cyclopentenyl, cyclohexenyl or cycloheptenyl. An exemplary multicyclic cycloalkenyl ring is norbornenyl.

As used herein, the term “derivative” in the context of proteinaceous agent (e.g., proteins, polypeptides, peptides, and antibodies) refers to a proteinaceous agent that comprises an amino acid sequence which has been altered by the introduction of amino acid residue substitutions, deletions, and/or additions. The term “derivative” as used herein also refers to a proteinaceous agent which has been modified, i.e., by the covalent attachment of any type of molecule to the proteinaceous agent. For example, but not by way of limitation, an antibody may be modified, e.g., by glycosylation, acetylation, pegylation, phosphorylation, amidation, derivatization by known protecting/blocking groups, proteolytic cleavage, linkage to a cellular ligand or other protein, etc. A derivative of a proteinaceous agent may be produced by chemical modifications using techniques known to those of skill in the art, including, but not limited to specific chemical cleavage, acetylation, formylation, metabolic synthesis in the presence of tunicamycin, etc. Further, a derivative of a proteinaceous agent may contain one or more non-classical amino acids. A derivative of a proteinaceous agent possesses a similar or identical function as the proteinaceous agent from which it was derived. The term “derivative” in the context of a proteinaceous agent also refers to a proteinaceous agent that possesses a similar or identical function as a second proteinaceous agent (i.e., the proteinaceous agent from which the derivative was derived) but does not necessarily comprise a similar or identical amino acid sequence of the second proteinaceous agent, or possess a similar or identical structure of the second proteinaceous agent. A proteinaceous agent that has a similar amino acid sequence refers to a second proteinaceous agent that satisfies at least one of the following: (a) a proteinaceous agent having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a second proteinaceous agent; (b) a proteinaceous agent encoded by a nucleotide sequence that hybridizes under stringent conditions to a nucleotide sequence encoding a second proteinaceous agent of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, or at least 150 contiguous amino acid residues; and (c) a proteinaceous agent encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding a second proteinaceous agent. A proteinaceous agent with similar structure to a second proteinaceous agent refers to a proteinaceous agent that has a similar secondary, tertiary or quaternary structure to the second proteinaceous agent. The structure of a proteinaceous agent can be determined by methods known to those skilled in the art, including but not limited to, peptide sequencing, X-ray crystallography, nuclear magnetic resonance, circular dichroism, and crystallographic electron microscopy. In a specific embodiment, a derivative is a functionally active derivative.

As used herein, the phrase “diagnostic agent” refers to any molecule, compound, and/or substance that is used for the purpose of diagnosing cancer. Non-limiting examples of diagnostic agents include antibodies, antibody fragments, or other proteins, including those conjugated to a detectable agent. As used herein, the term “detectable agents” refer to any molecule, compound and/or substance that is detectable by any methodology available to one of skill in the art. Non-limiting examples of detectable agents include dyes, gases, metals, or radioisotopes.

As used herein, the term “effective amount” refers to the amount of a therapy that is sufficient to result in the prevention of the development, recurrence, or onset of cancer and one or more symptoms thereof, to enhance or improve the prophylactic effect(s) of another therapy, reduce the severity, the duration of cancer, ameliorate one or more symptoms of cancer, prevent the advancement of cancer, cause regression of cancer, and/or enhance or improve the therapeutic effect(s) of another therapy. In an embodiment of the invention, the amount of a therapy is effective to achieve one, two or three or more results following the administration of one, two, three or more therapies: (1) a stabilization, reduction or elimination of the cancer stem cell population; (2) a stabilization, reduction or elimination in the cancer cell population; (3) a stabilization or reduction in the growth of a tumor or neoplasm; (4) an impairment in the formation of a tumor; (5) eradication, removal, or control of primary, regional and/or metastatic cancer; (6) a reduction in mortality; (7) an increase in disease-free, relapse-free, progression-free, and/or overall survival, duration, or rate; (8) an increase in the response rate, the durability of response, or number of patients who respond or are in remission; (9) a decrease in hospitalization rate, (10) a decrease in hospitalization lengths, (11) the size of the tumor is maintained and does not increase or increases by less than 10%, preferably less than 5%, preferably less than 4%, preferably less than 2%, (12) an increase in the number of patients in remission, (13) an increase in the length or duration of remission, (14) a decrease in the recurrence rate of cancer, (15) an increase in the time to recurrence of cancer, and (16) an amelioration of cancer-related symptoms and/or quality of life.

As used herein, the phrase “elderly human” refers to a human between 65 years old or older, preferably 70 years old or older.

As used herein, the term “enantiomer” or “enantiomeric” refers to a molecule that is non-superimposable on its mirror image and hence, is optically active wherein the enantiomer rotates the plane of polarized light in one direction and its mirror image rotates the plane of polarized light in the opposite direction.

As used herein, the phrase “human adult” refers to a human 18 years of age or older.

As used herein, the phrase “human child” refers to a human between 24 months of age and 18 years of age.

As used herein, the phrase “human infant” refers to a human less than 24 months of age, preferably less than 12 months of age, less than 6 months of age, less than 3 months of age, less than 2 months of age, or less than 1 month of age.

As used herein, the phrase “human patient” refers to any human, whether elderly, an adult, child or infant.

As used herein, the term “specifically binds to an antigen” and analogous terms refer to peptides, polypeptides, proteins, fusion proteins and antibodies or fragments thereof that specifically bind to an antigen or a fragment and do not specifically bind to other antigens. A peptide, polypeptide, protein, or antibody that specifically binds to an antigen may bind to other peptides, polypeptides, or proteins with lower affinity as determined by, e.g., immunoassays, BIAcore, or other assays known in the art. Antibodies or fragments that specifically bind to an antigen may be cross-reactive with related antigens. Preferably, antibodies or fragments that specifically bind to an antigen do not cross-react with other antigens. An antibody binds specifically to an antigen when it binds to the antigen with higher affinity than to any cross-reactive antigen as determined using experimental techniques, such as radioimmunoassays (RIAs) and enzyme-linked immunosorbent assays (ELISAs). See, e.g., Paul, ed., 1989, Fundamental Immunology, 2^{nd} ed., Raven Press, New York at pages 332-336 for a discussion regarding antibody specificity.

As used herein, the term “in combination” in the context of the administration of a therapy to a subject refers to the use of more than one therapy (e.g., prophylactic and/or therapeutic). The use of the term “in combination” does not restrict the order in which the therapies (e.g., a first and second therapy) are administered to a subject. A therapy can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, I week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, I week, 2 weeks, 3 weeks, 4 weeks, 5 weeks, 6 weeks, 8 weeks, or 12 weeks after) the administration of a second therapy to a subject which had, has, or is susceptible to cancer. The therapies are administered to a subject in a sequence and within a time interval such that the therapies can act together. In a particular embodiment, the therapies are administered to a subject in a sequence and within a time interval such that they provide an increased benefit than if they were administered otherwise. Any additional therapy can be administered in any order with the other additional therapy.

As used herein, the terms “manage,” “managing,” and “management” in the context of the administration of a therapy to a subject refer to the beneficial effects that a subject derives from a therapy (e.g., a prophylactic or therapeutic agent) or a combination of therapies, while not resulting in a cure of cancer. In certain embodiments, a subject is administered one or more therapies (e.g., one or more prophylactic or therapeutic agents) to “manage” cancer so as to prevent the progression or worsening of the condition.

As used herein, the term “marker” in the context of a cell or tissue (e.g. a normal or cancer cell or tumor) means any antigen, molecule or other chemical or biological entity that is specifically found in or on a tissue that it is desired to identify a particular tissue affected by a disease or disorder. In specific embodiments, the marker is a cell surface antigen that is differentially or preferentially expressed by specific cell types. For example, a leukemia cancer stem cell differentially expresses CD123 relative to a normal hematopoietic stem cell.

As used herein, the term “marker phenotype” in the context of a tissue (e.g., a normal or cancer cell or a tumor) means any combination of antigens (e.g., receptors and ligands), molecules or other chemical or biological entities that are specifically found in or on a tissue that it is desired to identify a particular tissue affected by a disease or disorder. In specific embodiments, the marker phenotype is a cell surface phenotype. In accordance with this embodiment, the cell surface phenotype may be determined by detecting the expression of a combination of cell surface antigens. Non-limiting examples of cell surface phenotypes of cancer stem cells of certain tumor types include CD34⁺/CD38⁻, CD34⁺/CD38⁻/CD123⁺, CD44⁺/CD24⁻, CD133⁺, CD34⁺/CD10^{-/CD}19⁻, CD138⁻/CD34⁻/CD19⁺, CD133⁺/RC2⁺, CD44⁺/α₂β₁^{hi}/CD133⁺, CLL-1, SLAMs, and other cancer stem cell surface phenotypes mentioned herein, as well as those that are known in the art.

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the terms “prevent”, “preventing” and “prevention” in the context of the administration of a therapy to a subject refer to the prevention or inhibition of the recurrence, onset, and/or development of a cancer or a symptom thereof in a subject resulting from the administration of a therapy (e.g., a prophylactic or therapeutic agent), or a combination of therapies (e.g., a combination of prophylactic or therapeutic agents). In some embodiments, such terms refer to one, two, three or more results following the administration of one or more therapies: (1) a stabilization, reduction or elimination of the cancer stem cell population, (2) a stabilization, reduction or elimination in the cancer cell population, (3) an increase in response rate, (4) an increase in the length or duration of remission, (5) a decrease in the recurrence rate of cancer, (6) an increase in the time to recurrence of cancer, and (7) an increase in the disease-free, relapse-free, progression-free, and/or overall survival of the patient. In specific embodiments, such terms refer to a stabilization, reduction or elimination of the cancer stem cell population.

As used herein, the term “predetermined reference range” refers to a reference range for the particular biological entity e.g., cancer stem cell, for a subject or a population of subjects. Each laboratory may establish its own reference range for each particular assay, or a standard reference range for each assay may be made available and used locally, regionally, nationally, or worldwide or may be patient-specific. In one specific embodiment, the term refers to a reference range for the amount of cancer stem cells in a patient (e.g., as determined by in vivo imaging) or a specimen from a patient. In another specific embodiment, the term refers to a reference range for the amount of cancer cells in a patient (e.g. as described by in vivo imaging) or a specimen from a patient.

As used herein, the phrase “prophylactic agent” refers to any molecule, compound, and/or substance that is used for the purpose of preventing cancer. Examples of prophylactic agents include, but are not limited to, proteins, immunoglobulins (e.g., multi-specific Igs, single chain Igs, Ig fragments, polyclonal antibodies and their fragments, monoclonal antibodies and their fragments), antibody conjugates or antibody fragment conjugates, peptides (e.g., peptide receptors, selectins), binding proteins, chemospecific agents, chemotoxic agents (e.g., anti-cancer agents), and small molecule drugs.

As used herein, the term “prophylactically effective regimen” refers to an effective regimen for dosing, timing, frequency and duration of the administration of one or more therapies for the prevention of cancer or a symptom thereof. In a specific embodiment, the regimen achieves one, two, or three or more of the following results: (1) a stabilization, reduction or elimination of the cancer stem cell population, (2) a stabilization, reduction or elimination in the cancer cell population, (3) an increase in response rate, (4) an increase in the length or duration of remission, (5) a decrease in the recurrence rate of cancer, (6) an increase in the time to recurrence of cancer, (7) an increase in the disease-free, relapse-free, progression-free, and/or overall survival of the patient, and (8) an amelioration of cancer-related symptoms and/or quality of life. As used herein, the term “racemic” refers to a mixture of equal parts of enantiomers and which is optically inactive.

As used herein, the term “resolution” refers to the separation or concentration or depletion of one of the two enantiomeric forms of a molecule.

As used herein, the term “small reduction”, in the context of a particular cell population (e.g., circulating endothelial cells and/or circulating endothelial progenitors) refers to less than a 30% reduction in the cell population (e.g., the circulating endothelial cell population and/or the circulating endothelial progenitor population).

As used herein, the term “stabilizing” and analogous terms, when used in the context of a cancer stem cell population or cancer cell population, refer to the prevention of an increase in the cancer stem cell population or cancer cell population, respectively. In other words, the amount of cancer stem cells or the amount of cancer cells that a cancer is composed of is maintained, and does not increase, or increases by less than 10%, preferably less than 5%.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

As used herein, the term “synergistic” refers to a combination of therapies which is more effective than the additive effects of any two or more single therapies. A synergistic effect of a combination of therapies permits the use of lower dosages of one or more of therapies and/or less frequent administration of said therapies to a subject. The ability to utilize lower dosages of therapies and/or to administer said therapies less frequently reduces the toxicity associated with the administration of said therapies to a subject without reducing the efficacy of said therapies in the prevention, treatment, and/or management of cancer. In addition, a synergistic effect can result in improved efficacy of therapeutic modalities in the prevention or treatment of cancer. Finally, the synergistic effect of a combination of therapies may avoid or reduce adverse or unwanted side effects associated with the use of any single therapy.

As used herein, the terms “subject” and “patient” are used interchangeably. As used herein, the term “subject” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is an elderly human. In another embodiment, the subject is a human adult. In another embodiment, the subject is a human child. In yet another embodiment, the subject is a human infant.

As used herein, the term “therapeutically effective regimen” refers to a regimen for dosing, timing, frequency, and duration of the administration of one or more therapies for the treatment and/or management of cancer or a symptom thereof. In a specific embodiment, the regimen achieves one, two, three, or more of the following results: (1) a stabilization, reduction or elimination of the cancer stem cell population; (2) a stabilization, reduction or elimination in the cancer cell population; (3) a stabilization or reduction in the growth of a tumor or neoplasm; (4) an impairment in the formation of a tumor; (5) eradication, removal, or control of primary, regional and/or metastatic cancer; (6) a reduction in mortality; (7) an increase in disease-free, relapse-free, progression-free, and/or overall survival, duration, or rate; (8) an increase in the response rate, the durability of response, or number of patients who respond or are in remission; (9) a decrease in hospitalization rate, (10) a decrease in hospitalization lengths, (11) the size of the tumor is maintained and does not increase or increases by less than 10%, preferably less than 5%, preferably less than 4%, preferably less than 2%, and (12) a increase in the number of patients in remission.

As used herein, the term “therapeutic agent” refers to any molecule, compound, and/or substance that is used for the purpose of treating and/or managing cancer. Examples of therapeutic agents include, but are not limited to, proteins, immunoglobulins (e.g., multi-specific Igs, single chain Igs, Ig fragments, polyclonal antibodies and their fragments, monoclonal antibodies and their fragments), antibody conjugates or antibody fragment conjugates, peptides (e.g., peptide receptors, selectins), binding proteins, chemospecific agents, chemotoxic agents (e.g., anti-cancer agents), radiation, chemotherapy, anti-angiogenic agents, and small molecule drugs. Therapeutic agents may be a(n) anti-angiogenesis therapy, targeted therapy, radioimmunotherapy, small molecule therapy, biologic therapy, epigenetic therapy, toxin therapy, differentiation therapy, pro-drug activating enzyme therapy, antibody therapy, chemotherapy, radiation therapy, hormonal therapy, immunotherapy, or protein therapy.

As used herein, the terms “therapies” and “therapy” can refer to any method(s), composition(s), and/or agent(s) that can be used in the prevention, treatment and/or management of a cancer or one or more symptoms thereof. In certain embodiments, the terms “therapy” and “therapies” refer to chemotherapy, radiation therapy, radioimmunotherapy, hormonal therapy, targeted therapy, toxin therapy, pro-drug activating enzyme therapy, protein therapy, antibody therapy, small molecule therapy, epigenetic therapy, demethylation therapy, histone deacetylase inhibitor therapy, differentiation therapy, antiangiogenic therapy, biological therapy including immunotherapy and/or other therapies useful in the prevention, management and/or treatment of a cancer or one or more symptoms thereof.

As used herein, the terms “treat”, “treatment”, and “treating” in the context of the administration of a therapy to a subject refer to the reduction or inhibition of the progression and/or duration of cancer, the reduction or amelioration of the severity of cancer, and/or the amelioration of one or more symptoms thereof resulting from the administration of one or more therapies. In specific embodiments, such terms refer to one, two or three or more results following the administration of one, two, three or more therapies: (1) a stabilization, reduction or elimination of the cancer stem cell population; (2) a stabilization, reduction or elimination in the cancer cell population; (3) a stabilization or reduction in the growth of a tumor or neoplasm; (4) a reduction in the formation of a tumor; (5) eradication, removal, or control of primary, regional and/or metastatic cancer; (6) a reduction in mortality; (7) an increase in disease-free, relapse-free, progression-free, or overall survival, duration, or rate; (8) an increase in the response rate, the durability of response, or number of patients who respond or are in remission; (9) a decrease in hospitalization rate, (10) a decrease in hospitalization lengths, and (11) the size of the tumor is maintained and does not increase or increases by less than 10%, preferably less than 5%, preferably less than 4%, preferably less than 2%. In certain embodiments, such terms refer to a stabilization or reduction in the cancer stem cell population. In some embodiments, such terms refer to a stabilization or reduction in the growth of cancer cells. In some embodiments, such terms refer to a stabilization or reduction in the cancer stem cell population and a reduction in the cancer cell population. In some embodiments, such terms refer to a stabilization or reduction in the growth and/or formation of a tumor. In some embodiments, such terms refer to the eradication, removal, or control of primary, regional, or metastatic cancer (e.g., the minimization or delay of the spread of cancer). In some embodiments, such terms refer to a reduction in mortality and/or an increase in survival rate of a patient population. In further embodiments, such terms refer to an increase in the response rate, the durability of response, or number of patients who respond or are in remission. In some embodiments, such terms refer to a decrease in hospitalization rate of a patient population and/or a decrease in hospitalization length for a patient population.

Concentrations, amounts, cell counts, percentages and other numerical values may be presented herein in a range format. It is to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

### 4. FIGURES

FIG. 1 shows a dose response curve of cord blood derived CD34⁺ cells in the presence of cantharidin and norcantharidin as measured by the XTT assay.

FIG. 2 shows a bar graph with cobblestone area counts of CD34⁺ cells obtained from a leukemia patient and normal cord blood in the presence of no drug (control) versus cantharidin (10 μM and 75 μM).

FIG. 3 shows a bar graph with cobblestone area counts of CD34⁺ cells obtained from a leukemia patient and normal cord blood in the presence of no drug (control) versus norcantharidin (10 μM and 75 μM).

FIG. 4 shows time course of MV4;11 leukemia cell viability in the presence of different concentrations of cantharidin.

FIG. 5 shows time course of MV4;11 leukemia cell viability in the presence of different concentrations of norcantharidin.

FIG. 6 shows a dose response curve of leukemia stem cells treated with cantharidin and standard chemotherapy (Ara-C and Daunorubicin) as measured by the Cobblestone Area Forming Cell Assay (CAFC).

### 5. DETAILED DESCRIPTION OF THE INVENTION

The present invention generally relates to methods for preventing, treating, and/or managing cancer, the methods comprising administering to a patient in need thereof a prophylactically effective regimen or a therapeutically effective regimen, the regimen comprising administering to the patient a compound of formula I, II, or III, or a pharmaceutically acceptable salt thereof (as described in Section 5.1 infra). In some embodiments, the regimen results in a reduction of the cancer stem cell population. While not being bound by any specific theory, a reduction in the amount of cancer stem cells or the elimination of cancer stem cells in the patient ultimately improves prospects for relapse-free cancer survival. In specific embodiments, the compounds of the invention demonstrate cytotoxicity against cancer stem cells in comparison with stem cells obtained from healthy subjects. In some embodiments the compounds of the invention demonstrate cytotoxicity against cancer stem cells at tolerable doses.

For clarity of disclosure, and not by way of limitation, the detailed description of the invention is divided into the subsections which follow.

5.1 Compounds of the Invention

The compounds of the invention are cantharidin and cantharidin analogs represented by compounds of formula I, II, or III,

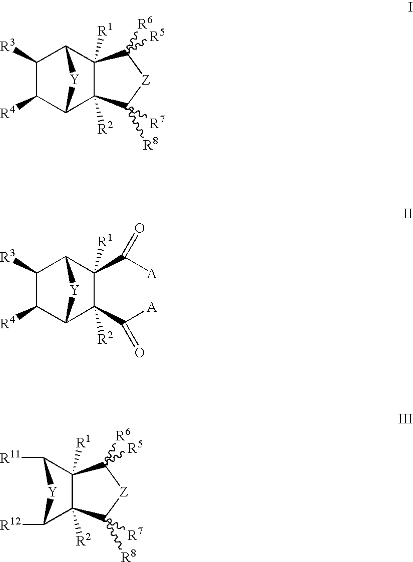

wherein

R¹ and R² are independently H or CH₃;

R³ and R⁴ are independently H, C₁-C₆ alkyl, aryl, or ara (C₁-C₁₀)alkyl; or together R³ and R⁴ form a bond (i.e., to form a cyclohexenyl ring);

R⁵, R⁶, R⁷, and R⁸ are independently H or OH, or R⁵ and R⁶, or R⁷ and R⁸ together with the carbon to which they are attached, form C═O;

R¹¹ and R¹² are independently H, C₁-C₁₀ alkyl, aryl, or ara(C₁-C₁₀)alkyl;

Y is O, N, or S;

A is OH or OR¹⁰, wherein R¹⁰ is C₁-C₆ alkyl;

Z is O, S, SR¹⁴, N—R⁹, CH₂OR¹², CHQ, or an amino acid;
R¹⁴ is C₁-C₁₀ alkyl, aryl, or ara(C₁-C₁₀)alkyl;
R⁹ is C₁-C₁₀ alkyl, H, OH, or Q;
R¹² is H or C₁-C₁₀ alkyl, aryl, or ara(C₁-C₁₀)alkyl;
wherein when Z is an amino acid, the α-amino group is a ring atom in a five-membered ring of formula I or III;
wherein Q is H or a moiety having the formula

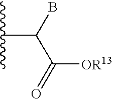

wherein R¹³ is C₁-C₁₀ alkyl or H; and B is (CH₂)ₙW, CH═CH—W, or CH₂OW, wherein W is an ionisable residue; or

a pharmaceutically acceptable salt thereof.

In certain embodiments, the compounds of the invention are represented by the compound of formula I or II,

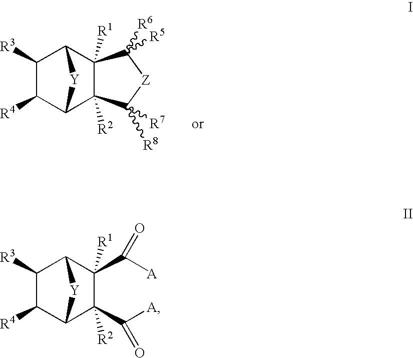

wherein

R¹ and R² are independently H or CH₃;

R³ and R⁴ are independently H, C₁-C₆ alkyl, or aryl; or together R³ and R⁴ form a bond (i.e., to form a cyclohexenyl ring);

R⁵, R⁶, R⁷, and R⁸ are independently H or OH, or R⁵ and R⁶, or R⁷ and R⁸ together with the carbon to which they are attached, form C═O;

Y is O, N, or S;

Z is O, S, NOH, or N—R⁹, wherein R⁹ is C₁-C₆ alkyl or H; and

A is OH or OR¹⁰, wherein R¹⁰ is C₁-C₆ alkyl; or

a pharmaceutically acceptable salt thereof.

In some embodiments, the compounds of the invention are represented by the compound of formula I or II,

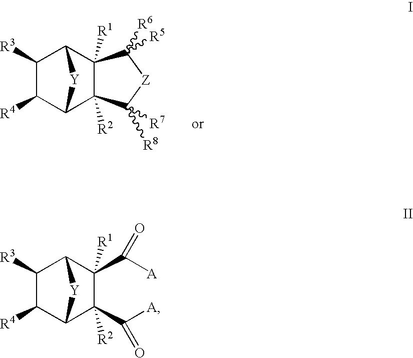

wherein

R¹ and R² are independently H or CH₃;

R³ and R⁴ are independently H, C₁-C₆ alkyl, or aryl; or together R³ and R⁴ form a bond (i.e., to form a cyclohexenyl ring);

R⁵, R⁶, R⁷, and R⁸ are independently H or OH, or R⁵ and R⁶, or R⁷ and R⁸ together with the carbon to which they are attached, form C═O;

Y is O, N, or S;

Z is O or S; and

A is OH or OR¹⁰, wherein R¹⁰ is C₁-C₆ alkyl; or

a pharmaceutically acceptable salt thereof.

In some embodiments, the compounds of the invention have the formula I. In certain embodiments of the compounds of the formula I, Y and Z are both O. In specific embodiments of the compounds of the formula I, Y and Z are both O, R¹ and R² are H or CH₃, R³ and R⁴ are both H, R⁵ and R⁶ together with the carbon to which they are attached are C═O, and R⁷ and R⁸ together with the carbon to which they are attached are C═O, so as to form a compound of the formula IA

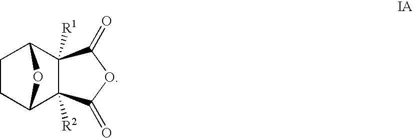

In one embodiment of the compound of the formula IA, R¹ and R² are both CH₃. In another embodiment of the compound of the formula IA, R¹ and R² are both H.

In some embodiments of the compounds of the invention having the formula I, Y is O, Z is NOH, R¹ and R² are CH₃, R³ and R⁴ are both H, R⁵ and R⁶ together with the carbon to which they are attached are C═O, and R⁷ and R⁸ together with the carbon to which they are attached are C═O, so as to form hydrocantharidimide.

In some embodiments, the compounds of the invention have the formula II. In certain embodiments of the compound of the formula II, Y is O. In specific embodiments of the compound of the formula II, Y is O, R¹ and R² are H or CH₃ so as to form a compound of the formula IIA

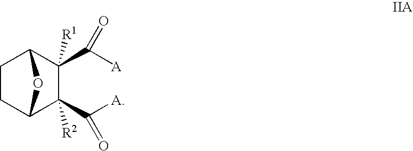

In some embodiments of the compounds of the formula IIA, R¹ and R² are both CH₃. In other embodiments of the compounds of the formula IIA, R¹ and R² are both H.

In some embodiments of the compounds of the formula IIA, A is OH. In other embodiments of the compounds of the formula IIA, A is OR¹⁰, wherein R¹⁰ is C₁-C₆ alkyl. In a specific embodiment of the compounds of the formula IIA, R¹ and R² are both CH₃, and A is OH. In a specific embodiment of the compounds of the formula IIA, R¹ and R² are both CH₃, and A is O⁻, wherein the compound is in the form of a pharmacologically acceptable salt having a counterion, e.g., Na⁺.

In another specific embodiment of the compounds of the formula IIA, R¹ and R² are both H, and A is OH. In another specific embodiment of the compounds of the formula IIA, R¹ and R² are both H, and A is O⁻, wherein the compound is in the form of a pharmacologically acceptable salt having a counterion, e.g., Na⁺.

In certain embodiments, compounds of the invention are represented by formula II, wherein A is independently OH or NR¹¹R¹².

R¹¹ or R¹² are independently H, substituted or unsubstituted C₁₋₈cycloalkyl, R¹¹R¹²N—C₁₋₈alkyl, NR¹¹R¹², substituted or unsubstituted aryl-C₁₋₈-alkyl, or substituted or unsubstituted heteroaryl-C₁₋₈alkyl. Furthermore the R¹¹ or R¹² are independently H or substituted or unsubstituted aryl.
R¹¹ and R¹² together with the nitrogen to which they are attached may form a substituted or unsubstituted saturated heterocycle, wherein one CH₂ group in the saturated heterocycle may be replaced by O, NR¹⁰, S, S(═O) or S(═O)₂. R¹, R², R³ and R⁴ are defined as in previous embodiments.

In an additional embodiment compounds of the invention are represented by formula II, wherein R³ and R⁴ are H, and A, R¹, R², R¹⁰, R¹¹ and R¹² are as described above.

In an additional embodiment compounds of the invention are represented by formula II, wherein R¹, R², R³, and R⁴ are H, and A, R¹⁰, R¹¹ and R¹² are as described above.

In a specific embodiment the compound of the invention is represented by formula II, wherein A is independently OH or NR¹¹R¹²; R¹ and R² are methyl; R³ and R⁴ are H; and R¹¹ and R¹² together with the nitrogen to which they are attached form a morphine ring.

In a specific embodiment compounds of the invention are represented by formula II, wherein:

A is independently OH or NR¹¹R¹²; R¹, R², R³, and R⁴ are H and R¹¹ and R¹² together with the nitrogen to which they are attached form moieties represented by the following list:

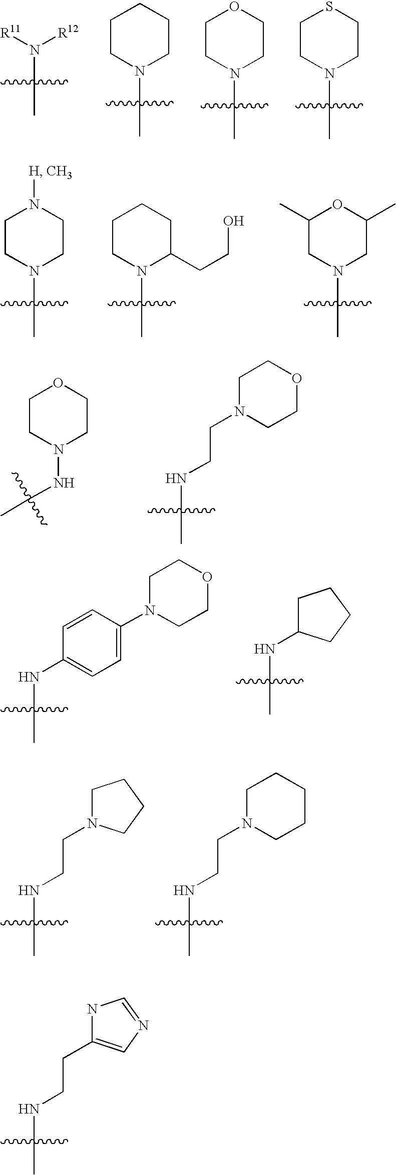

In certain embodiments compounds of the invention are represented by formula I, wherein:

### Z is NR¹³;

R¹³ is a substituted or unsubstituted aryl or substituted or unsubstituted heteroaryl; and
R¹, R², R³, R⁴, R⁶, R⁷, R⁸ and Y are as defined above.

In an additional embodiment, compounds of the invention are represented by formula I, wherein:

### Z is NR¹³;

R¹³ is a substituted or unsubstituted 8, 9, or 10 membered unsaturated heterobicyclic system with one or more heteroatoms independently selected from N, O and S; and
R¹, R², R³, R⁴, R⁵, R⁶, R⁷, R⁸ and Y are as defined above.

In an additional embodiment, compounds of the invention are represented by formula I, wherein:

### Z is NR¹³.

R¹ and R² are methyl;

### R³ and R⁴ are H;

R⁵ and R⁶ together are C═O as well as R⁷ and R⁸ together are C═O;

### Y is O; and

R¹³ is a substituted or unsubstituted 8, 9 or 10 membered unsaturated heterobicyclic system with one or more heteroatoms independently selected from N, O and S. For instance R¹³ may be benzothiazol-2-yl, 6-OMe-benzothiazol-2-yl, 6-Me-benzothiazol-2-yl, or 6-trifluoromethoxy-benzothiazol-2-yl.

In an additional embodiment, compounds of the invention are represented by formula I, wherein:

### Z is NR¹³

R¹ and R² are methyl;

### R³ and R⁴ are H;

R⁵ and R⁶ together are C═O as well as R⁷ and R⁸ together are C═O;

### Y is O; and

R¹³ is a substituted or unsubstituted 5 to 6 membered unsaturated heterocyclic system with one to three heteroatoms independently selected from N, O and S. For instance R¹³ may be 2-mercapto-1,3,4-thiadiazole-2-yl.

In a specific embodiment, compounds of the invention are represented by formula I, wherein:

### Z is NR¹³

R¹ and R² are methyl;

### R³ and R⁴ are H;

R⁵ and R⁶ together are C═O as well as R⁷ and R⁸ together are C═O;

### Y is O; and

R¹³ is as follows:

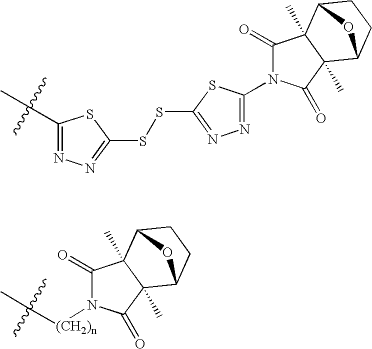

wherein n=2, 3, 4 or 5.

In a specific embodiment, compounds of the invention are represented by formula I, wherein:

R¹ is CH₂OH or methyl and R² is methyl;

### R³ and R⁴ are H;

R⁵ and R⁶ as well as R⁷ and R⁸ are C═O;

### Y is O; and

### Z=NH.

In other embodiments compounds of the invention are represented by formula I, wherein:

R¹ and R² are independently H or CH₃;
R³ and R⁴ are independently H, C₁-C₆ alkyl, or aryl; or together R³ and R⁴ form a bond (i.e., to form a cyclohexenyl ring);
R⁵, R⁶, R⁷, and R⁸ are independently H or OH, or R⁵ and R⁶, or R⁷ and R⁸ together with the carbon to which they are attached, form C═O;

### Y is O, N, or S;

Z is PtL₂, wherein each L is ammonia or together form a bidentate bis amino ligand such as a substituted or unsubstituted cyclohexane-1,2-diamine; or
a pharmaceutically acceptable salt thereof.

In a specific embodiment compounds of the invention are represented by formula IV, wherein:

### Z is PtL₂;

### R¹ and R² are CH₃;

### R³ and R⁴ are H;

R⁵ and R⁶, or R⁷ and R⁸ together with the carbon to which they are attached, form C═O;

### Y is O; and

L and L together represent a bidentate bis amino ligand selected from (1S,2S)-cyclohexane-1,2-diamine, (1R,2R)-cyclohexane-1,2-diamine, or cis-cyclohexane-1,2-diamine.

Any of the above-described compounds of the formulas I (including IA), formula II (including IIA), and formula III may be used in the prophylactic and therapeutic methods described in Section 5.3.

Some of the compounds disclosed herein may contain one or more asymmetric centers, and may thus give rise to enantiomers, diastereomers, and other stereoisomeric forms. The present invention is meant to encompass all such possible forms as well as their racemic and resolved forms and mixtures thereof (e.g., an enantiomeric mixture enriched for one enantiomer). A compound of the invention having one or more asymmetric centers can be in the form of an optical isomer or a diastereomer. Purified optical isomers can be isolated by known techniques such as chiral chromatography, or by formation of diastereomeric salts from an optically active acid or base. In other embodiments, optically pure isomers can be obtained by synthesis from optically pure starting materials.

The invention disclosed herein is meant to encompass all pharmaceutically acceptable salts thereof of the disclosed compounds. The pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, sulfate, phosphate and the like; organic acid salts such as formate, acetate, trifluoroacetate, maleate, fumarate, tartrate and the like; sulfonates such as methanesulfonate, benzenesulfonate, p-toluenesulfonate, and the like; amino acid salts such as arginate, asparaginate, glutamate and the like.

The invention disclosed herein is also meant to encompass all prodrugs of the disclosed compounds. It will be appreciated by those skilled in the art that certain protected derivatives of the compounds of the invention, such as formula I, II or III, which may be made prior to a final deprotection stage, may not possess pharmacological activity as such, but may, in certain instances, be administered orally or parenterally and thereafter metabolised in the body to form compounds of the invention which are pharmacologically active. Such derivatives may therefore be described as “prodrugs”. All such prodrugs of compounds of the invention are included within the scope of the invention. Examples of suitable pro-drugs for the compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter I (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within compounds of the invention.

In a specific embodiment prodrugs are considered to be any covalently bonded carriers which release the active drug in vivo.

The invention disclosed herein is also meant to encompass the in vivo metabolic products of the disclosed compounds. Such products may result, for example, from the oxidation, reduction, hydrolysis, amidation, esterification and the like of the administered compound, primarily due to enzymatic processes. Accordingly, the invention includes compounds produced by a process comprising contacting a compound of this invention with a mammal for a period of time sufficient to yield a metabolic product thereof. Such products typically are identified by preparing a radiolabeled compound of the invention, administering it parenterally in a detectable dose to an animal such as rat, mouse, guinea pig, monkey, or to man, allowing sufficient time for metabolism to occur and isolating its conversion products from the urine, blood or other biological samples.

The invention disclosed herein is also meant to encompass the disclosed compounds being isotopically-labeled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

In certain embodiments, the compounds of the invention may be covalently bound to antibodies or other agents that target cancer stem cells. For instance, the compounds of the invention can be covalently bound to an antibody that binds to a cell surface antigen present on a cancer stem cell such as CD123. Other non limiting examples of cell surface antigens present on a cancer stem cell include CD44, CD34, CD133, CD19, CD38, CD20, CD123, CLL-1, RC2 and α₂β₁.

The compounds of the invention can be covalently bound to an agent, e.g., an antibody, using known conjugation techniques. For example, one end of a cross-linking agent can be bound to a chemical moiety (e.g., a carboxylic acid moiety) on the compound of the invention, while the other end of the cross-linking agent is bound to a site on an antibody. Such bifunctional cross-linking agents and their use in preparing conjugated antibodies are known and discussed in Hermanson, G. T. Bioconjugate Techniques, Academic, San Diego, Calif., 1996; pp. 494-527.

5.1.1 Syntheses of the Compounds of the Invention

The compounds of the invention may be obtained from natural sources, from chemical syntheses and from semi-synthetic processes.

Cantharidin, itself, can be extracted from mylabris (dried body of the Chinese blister beetle). Moreover, total syntheses of cantharidin have been completed. See Stork et al., J. Am. Chem. Soc. 1953, 75, 384-392, and Dauben et al., J. Am. Chem. Soc. 1980, 102, 6892-6894.

Compounds of the invention can be synthesized from known starting materials using known synthetic reactions. In the case of compounds of the formula I, certain of the compounds of the invention can be obtained using Diels-Alder reactions of maleic acid derivatives. For instance, Scheme I exemplifies one embodiment of the invention wherein certain compounds of the invention D-1, D-2, and D-3 can be formed (wherein R³ and R⁴ are as described above). Maleic anhydride B-1 and a substituted/unsubstituted furan A-1 are combined using a Diels-Alder reaction to give intermediate C-1. In preparing certain C-1 intermediates, elevated pressures may be applied to enhance conversion of the starting materials.

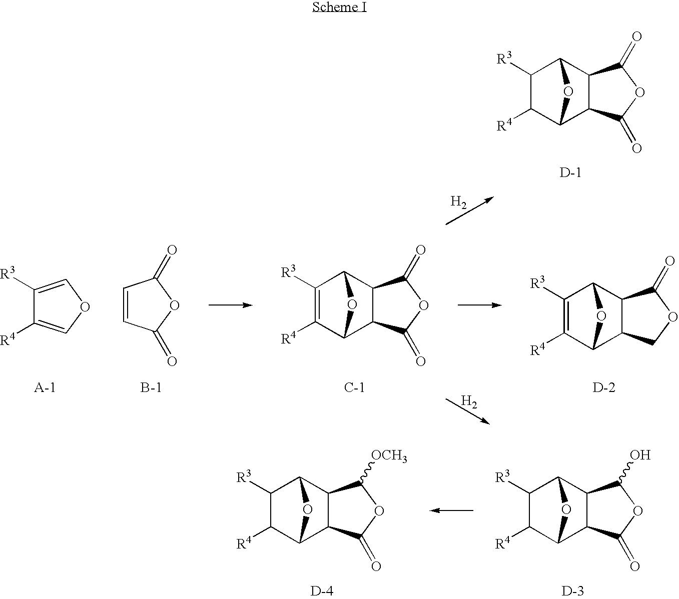

Catalytic reduction (e.g., 10% palladium on carbon) of intermediate C-1 in acetone affords a compound of the formula D-1. Reduction of intermediate C-1 using sodium borohydride in the presence of hydrochloric acid affords intermediate D-2. Catalytic reduction (e.g., 10% palladium on carbon) of intermediate C-1 in ethanol affords a compound of the formula D-3. Reaction of D-3 in methanol in the presence of acid (e.g., p-toluenesulfonic acid) provides compounds of the formula D-4. Price et al. also describes the synthesis of certain compounds of the formula I or II in Price et al., “Radiosensitization of tumour cells by cantharidin and some analogues,” Int. J. Radiat. Biol. 80:(4) 269-279 (2004).

Furthermore, the synthesis of certain of the compounds of the formulas I, II and III are described in McCluskey et al., U.S. Patent Application Publication Nos. 2004/0209934 A1 and 2004/0110822 A1; McCluskey et al. Bioorg. Med. Chem. Lett. 17 (2007), 3392-2297; On Tang et al. Int. J. Mol. Med. 17 (2006), 151-157; On Tang et al. Bioorg. Med. Chem. Lett. 17 (2007), 1155-1159; On Tang et al. Int. J. Mol. Med. 18 (2006), 1217-1221; On Tang et al. Int. J. Mol. Med. 18 (2006), 375-379; Noda et al. Chem. Pharm. Bull 55 (2007), 92-94; Ho et al. Bioorg. Med. Chem. Lett. 16 (2006), 1686-1691; the disclosures of all of which are hereby incorporated by reference in their entireties.

Other compounds can be obtained by semi-synthetic procedures where a naturally occurring intermediate, e.g., cantharidin, is modified synthetically. For instance, as shown in Scheme II, cantharidin, obtained from a natural source, can be modified by reaction with either sodium hydroxide, hydroxylamine, or methylamine, respectively, to provide certain compounds of the invention. See, Wang et al., “Medical uses of Mylabris in ancient China and recent studies,” J. of Ethnopharmacology, 26: 147-162 (1989).

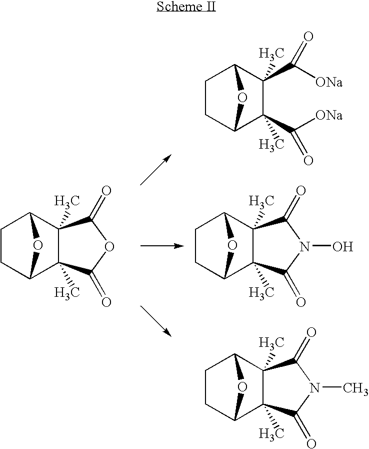

Finally, certain of the compounds, such as cantharidin and norcantharidin can be purchased from commercial sources, such as from Sigma-Aldrich Co., St. Louis, Mo.

5.2 Pharmaceutical Compositions and Routes of Administration

The present invention provides composition comprising a compound of the invention. In particular, the invention provides a pharmaceutical composition comprising an effective amount of a compound of the invention and a pharmaceutically acceptable carrier or vehicle. In a specific embodiment, a pharmaceutical composition comprises an effective amount of a compound of the invention and a pharmaceutically acceptable carrier or vehicle. The pharmaceutical compositions are suitable for veterinary and/or human administration.

The pharmaceutical compositions of the present invention can be in any form that allows for the composition to be administered to a subject, said subject preferably being an animal, including, but not limited to a human, mammal, or non-human animal, such as a cow, horse, sheep, pig, fowl, cat, dog, mouse, rat, rabbit, guinea pig, etc., and is more preferably a mammal, and most preferably a human.

The compositions of the invention can be in the form of a solid, liquid or gas (aerosol). Typical routes of administration may include, without limitation, oral, topical, parenteral, sublingual, rectal, vaginal, ocular, intradermal, intratumoral, intracerebral, intrathecal, and intranasal. Parenteral administration includes subcutaneous injections, intravenous, intramuscular, intraperitoneal, intrapleural, intrasternal injection or infusion techniques. In a specific embodiment, the compositions are administered parenterally. In a more specific embodiment, the compositions are administered intravenously. Pharmaceutical compositions of the invention can be formulated so as to allow a compound of the invention to be bioavailable upon administration of the composition to a subject. Compositions can take the form of one or more dosage units, where, for example, a tablet can be a single dosage unit, and a container of a compound of the invention in aerosol form can hold a plurality of dosage units.

Materials used in preparing the pharmaceutical compositions can be non-toxic in the amounts used. It will be evident to those of ordinary skill in the art that the optimal dosage of the active ingredient(s) in the pharmaceutical composition will depend on a variety of factors. Relevant factors include, without limitation, the type of subject (e.g., human), the overall health of the subject, the type of cancer the subject is in need of treatment of, the use of the composition as part of a multi-drug regimen, the particular form of the compound of the invention, the manner of administration, and the composition employed.

The pharmaceutically acceptable carrier or vehicle may be particulate, so that the compositions are, for example, in tablet or powder form. The carrier(s) can be liquid, with the compositions being, for example, an oral syrup or injectable liquid. In addition, the carrier(s) can be gaseous, so as to provide an aerosol composition useful in, e.g., inhalatory administration.

The term “carrier” refers to a diluent, adjuvant or excipient, with which a compound of the invention is administered. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. In one embodiment, when administered to a subject, the compounds of the invention and pharmaceutically acceptable carriers are sterile. Water is a preferred carrier when the compound of the invention is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The composition may be intended for oral administration, and if so, the composition is preferably in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more of the following can be present: binders such as ethyl cellulose, carboxymethylcellulose, microcrystalline cellulose, or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin, a flavoring agent such as peppermint, methyl salicylate or orange flavoring, and a coloring agent.

When the pharmaceutical composition is in the form of a capsule, e.g., a gelatin capsule, it can contain, in addition to materials of the above type, a liquid carrier such as polyethylene glycol, cyclodextrin or a fatty oil.

The pharmaceutical composition can be in the form of a liquid, e.g., an elixir, syrup, solution, emulsion or suspension. The liquid can be useful for oral administration or for delivery by injection. When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included.

The liquid compositions of the invention, whether they are solutions, suspensions or other like form, can also include one or more of the following: sterile diluents such as water for injection, saline solution, preferably physiological saline, Ringer's solution, isotonic sodium chloride, fixed oils such as synthetic mono or diglycerides which can serve as the solvent or suspending medium, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. A parenteral composition can be enclosed in an ampoule, a disposable syringe or a multiple-dose vial made of glass, plastic or other material. Physiological saline is a preferred adjuvant. An injectable composition is preferably sterile.

The pharmaceutical compositions comprise an effective amount of a compound of the invention such that a suitable dosage will be obtained (see Section 5.3.1, infra, for suitable dosages). Typically, this amount is at least 0.01% of a compound of the invention by weight of the composition. When intended for oral administration, this amount can be varied to be between 0.1% and 80% by weight of the composition. Preferred oral compositions can comprise from between 4% and 50% of the compound of the invention by weight of the composition. Preferred compositions of the present invention are prepared so that a parenteral dosage unit contains from between 0.01% and 2% by weight of the compound of the invention.

The compounds of the invention can be administered by any convenient route, for example, by infusion or bolus injection, by absorption through epithelial or mucocutaneous linings (e.g., oral mucosa, rectal and intestinal mucosa, etc.). Administration can be systemic or local. Various delivery systems are known, e.g., microparticles, microcapsules, capsules, etc., and may be useful for administering a compound of the invention. In certain embodiments, more than one compound of the invention is administered to a subject. Methods of administration may include, but are not limited to, oral administration and parenteral administration; parenteral administration including, but not limited to, intradermal, intramuscular, intraperitoneal, intravenous, subcutaneous; intranasal, epidural, sublingual, intranasal, intracerebral, intraventricular, intrathecal, intravaginal, transdermal, rectally, by inhalation, or topically such as to the ears, nose, eyes, or skin. The preferred mode of administration is left to the discretion of the practitioner, and will depend in-part upon the site of the medical condition (such as the site of cancer, a cancerous tumor or a pre-cancerous condition).

In one embodiment, the compounds of the invention are administered parenterally. In a specific embodiment, the compounds of the invention are administered intravenously.

In specific embodiments, it can be desirable to administer one or more compounds of the invention locally to the area in need of treatment. This can be achieved, for example, and not by way of limitation, by local infusion during surgery; topical application, e.g., in conjunction with a wound dressing after surgery; by injection; by means of a catheter; by means of a suppository; or by means of an implant, the implant being of a porous, non-porous, or gelatinous material, including membranes, such as silastic membranes, or fibers. In one embodiment, administration can be by direct injection at the site (or former site) of a cancer, tumor, or precancerous tissue. In certain embodiments, it can be desirable to introduce one or more compounds of the invention into the central nervous system by any suitable route, including intraventricular and intrathecal injection. Intraventricular injection can be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the compounds of the invention can be formulated as a suppository, with traditional binders and carriers such as triglycerides.

In yet another embodiment, the compounds of the invention can be delivered in a controlled release system. In one embodiment, a pump can be used (see Sefton, CRC Crit. Ref: Biomed. Eng. 1987, 14, 201; Buchwald et al., Surgery 1980, 88: 507; Saudek et al., N. Engl. J. Med. 1989, 321: 574). In a specific embodiment aforementioned pump may be, but is not limited to, an insulin-like pump. In another embodiment, polymeric materials can be used (see Medical Applications of Controlled Release, Langer and Wise (eds.), CRC Pres., Boca Raton, Fla., 1974; Controlled Drug Bioavailability, Drug Product Design and Performance, Smolen and Ball (eds.), Wiley, New York, 1984; Ranger and Peppas, J Macromol. Sci. Rev. Macromol. Chem. 1983, 23, 61; see also Levy et al., Science 1985, 228, 190; During et al., Ann. Neurol., 1989, 25, 351; Howard et al., J. Neurosurg., 1989, 71, 105). In yet another embodiment, a controlled-release system can be placed in proximity of the target of the compounds of the invention, e.g., the brain, thus requiring only a fraction of the systemic dose (see, e.g., Goodson, in Medical Applications of Controlled Release, supra, vol. 2, 1984, pp. 115-138). Other controlled-release systems discussed in the review by Langer (Science 1990, 249, 1527-1533) can be used.

In another embodiment, polymeric materials can be used to achieve controlled or sustained release of the compounds of the invention (see, e.g., U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable carrier is a capsule (see e.g., U.S. Pat. No. 5,698,155). Other examples of suitable pharmaceutical carriers are described in Remington's Pharmaceutical Sciences by E. W. Martin.

Sustained or directed release compositions that can be formulated include, but are not limited to, compounds of the invention protected with differentially degradable coatings, e.g., by microencapsulation, multiple coatings, etc. It is also possible to freeze-dry the compositions and use the lyophilizates obtained, for example, for the preparation of products for injection.

In a preferred embodiment, the compounds of the invention are formulated in accordance with routine procedures as a pharmaceutical composition adapted for intravenous administration to animals, particularly human beings. Typically, the carriers or vehicles for intravenous administration are sterile isotonic aqueous buffer solutions. Where necessary, the compositions can also include a solubilizing agent. Compositions for intravenous administration can optionally comprise a local anaesthetics such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachet indicating the quantity of active agent. Where a compound of the invention is to be administered by infusion, it can be dispensed, for example, with an infusion bottle containing sterile pharmaceutical grade water or saline. Where the compound of the invention is administered by injection, an ampoule of sterile water for injection or saline can be provided so that the ingredients can be mixed prior to administration.

Compositions for oral delivery can be in the form of tablets, lozenges, aqueous or oily suspensions, granules, powders, emulsions, capsules, syrups, or elixirs, for example. Orally administered compositions can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving complex are also suitable for orally administered compositions of the invention. In these later platforms, fluid from the environment surrounding the capsule is imbibed by the driving complex, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard carriers such as mannitol, lactose, starch, magnesium stearate, sodium saccharine, cellulose, magnesium carbonate, etc. Such carriers are preferably of pharmaceutical grade.

The pharmaceutical compositions of the invention can be intended for topical administration, in which case the carrier can be in the form of a solution, emulsion, ointment or gel base. The base, for example, can comprise one or more of the following: petrolatum, lanolin, polyethylene glycols, beeswax, mineral oil, diluents such as water and alcohol, and emulsifiers and stabilizers. Thickening agents can be present in a composition for topical administration. If intended for transdermal administration, the composition can be in the form of a transdermal patch or an iontophoresis device. Topical formulations can comprise a concentration of a compound of the invention of from between 0.01% and 10% w/v (weight per unit volume of composition).

The compositions can include various materials that modify the physical form of a solid or liquid dosage unit. For example, the composition can include materials that form a coating shell around the active ingredients. The materials that form the coating shell are typically inert, and can be selected from, for example, sugar, shellac, and other enteric coating agents. Alternatively, the active ingredients can be encased in a gelatin capsule.

The compositions can consist of gaseous dosage units, e.g., it can be in the form of an aerosol. The term aerosol is used to denote a variety of systems ranging from those of colloidal nature to systems consisting of pressurized packages. Delivery can be by a liquefied or compressed gas or by a suitable pump system that dispenses the active ingredients. Aerosols of the compositions can be delivered in single phase, bi-phasic, or tri-phasic systems in order to deliver the composition. Delivery of the aerosol includes the necessary container, activators, valves, subcontainers, spacers and the like, which together can form a kit. Preferred aerosols can be determined by one skilled in the art, without undue experimentation.

Whether in solid, liquid or gaseous form, the compositions of the present invention can comprise an additional active agent selected from among those including, but not limited to, an additional prophylactic agent, an additional therapeutic agent, an antiemetics agent, a hematopoietic colony stimulating factor, an adjuvant therapy, a vaccine or other immune stimulating agent, an antibody/antibody fragment-based agent, an anti-depressant and an analgesic agent. For instance in a particular embodiment, the pharmaceutical composition comprises a compound of the invention, an additional agent, and a pharmaceutically acceptable carrier or vehicle.

The pharmaceutical compositions can be prepared using methodology well known in the pharmaceutical art. For example, a composition intended to be administered by injection can be prepared by combining a compound of the invention with water so as to form a solution. A surfactant can be added to facilitate the formation of a homogeneous solution or suspension. Surfactants are complexes that can non-covalently interact with a compound of the invention so as to facilitate dissolution or homogeneous suspension of the compound of the invention in the aqueous delivery system.

In one embodiment, the pharmaceutical compositions of the present invention may comprise one or more known therapeutically active agents.

5.3 Prophylactic and Therapeutic Uses

Cancer or a neoplastic disease, including, but not limited to, neoplasms, tumors, metastases, or any disease or disorder characterized by uncontrolled cell growth, can be treated, suppressed, delayed, managed, inhibited or prevented by administering to a subject in need thereof a prophylactically effective regimen or a therapeutically effective regimen, the regimen comprising administering to the patient a compound of the invention. The invention as it applies to cancer encompasses the treatment, suppression, delaying, management, inhibiting of growth and/or progression, and prevention of cancer or neoplastic disease as described herein.

In one embodiment, the compounds of the invention are administered as monotherapy for the prevention, treatment, and/or management of cancer.

One aspect of the invention relates to a method of preventing, treating, and/or managing cancer in a patient (e.g., a human patient), the method comprising administering to the patient a prophylactically effective regimen or a therapeutically effective regimen, the regimen comprising administering to the patient a compound of the invention or a composition of the invention, wherein the patient has been diagnosed with cancer.

In one embodiment, the cancer is a hematologic cancer. For instance, the cancer can be leukemia, lymphoma or myeloma or myelodysplastic syndrome. In another embodiment, the cancer is a solid tumor.

In one embodiment of this aspect, the patient has received or is receiving another therapy. In another embodiment of this aspect, the patient has not previously received a therapy for the prevention, treatment, and/or management of the cancer.

The medical practitioner can diagnose the patient using any of the conventional cancer screening methods including, but not limited to physical examination (e.g., prostate examination, breast examination, lymph nodes examination, abdominal examination, skin surveillance), visual methods (e.g., colonoscopy, bronchoscopy, endoscopy), PAP smear analyses (cervical cancer), stool guaiac analyses, blood tests (e.g., complete blood count (CBC) test, prostate specific antigen (PSA) test, carcinoembryonic antigen (CEA) test, cancer antigen (CA)-125 test, alpha-fetoprotein (AFP)), karyotyping analyses, bone marrow analyses (e.g., in cases of hematological malignancies), histology, cytology, a sputum analysis and imaging methods (e.g., computed tomography (CT), magnetic resonance imaging (MRI), ultrasound, X-ray imaging, mammography, bone scans).

Another aspect of the invention relates to a method of preventing, treating, and/or managing a solid tumor in a patient (e.g., a human patient), the method comprising administering to a patient in need thereof a prophylactically effective regimen or a therapeutically effective regimen, the regimen comprising administering to the patient a compound or composition of the invention wherein the patient has been diagnosed with a solid tumor, and wherein the patient has undergone a primary therapy that may reduce the bulk of the tumor. In this case the primary therapy that may reduce the tumor bulk size is preferably a therapy other than a compound or composition of the invention. In specific embodiment of this aspect, the solid tumor is fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, melanoma, neuroblastoma, or retinoblastoma.

Another aspect of the invention relates to a method of preventing, treating, and/or managing cancer, the method comprising administering to a patient in need thereof a prophylactically effective regimen or a therapeutically effective regimen, the regimen comprising administering to the patient a compound of formula I, II or III (as described above), or a pharmaceutically acceptable salt thereof wherein the patient received another therapy. In some embodiments, the prior therapy is, for example, chemotherapy, radioimmunotherapy, hormonal therapy, small molecule therapy, toxin therapy, prodrug-activating enzyme therapy, protein therapy, antibody therapy, surgical therapy, biologic therapy, immunotherapy, anti-angiogenic therapy, epigenetic therapy, demethylation therapy, histone deacetylase inhibitor therapy, differentiation therapy, radiation therapy, or any combination thereof.

In some embodiments, the prior therapy has failed in the patient. In some embodiments, the therapeutically effective regimen comprising administration of a compound of formula I, II or III is administered to the patient immediately after patient has undergone the prior therapy. For instance, in certain embodiments, the outcome of the prior therapy may be unknown before the patient is administered a compound of the formula I, II or III.

In certain embodiments of the invention, conventional chemotherapy and the therapeutically effective regimen comprising administration of a compound of formula I, II, and III of the invention may be used sequentially. In a specific aspect of this embodiment, the patient's leukemia blasts are first reduced by use of conventional chemotherapy, followed by a regimen comprising administration of an effective amount of a compound of the invention for a time sufficient to significantly impair or eradicate cancer stem cells.

Another aspect of the invention relates to a method of preventing cancer in a patient (e.g., a human patient), the method comprising administering to a patient in need thereof a prophylactically effective regimen or a therapeutically effective regimen, the regimen comprising administering to the patient a compound or composition of the invention, wherein the cancer in the patient has entered remission. In some embodiments of this aspect, through administration of a prophylactically effective regimen or a therapeutically effective regimen, the medical practitioner can effectively cure the cancer, or prevent its reoccurrence.

Another aspect of the invention relates to a method of preventing, treating, and/or managing a solid tumor in a patient (e.g., a human patient), the method comprising administering to a patient in need thereof a prophylactically effective regimen or a therapeutically effective regimen, the regimen comprising administering to the patient a compound or composition of the invention, wherein the compound or composition of the invention is administered at a dose that is lower than the maximum tolerated dose (MTD) over a period of three months, four months, six months, nine months, 1 year, 2 years, 3 years, 4 years or more.

Another aspect of the invention relates to a method of preventing, treating, and/or managing a solid tumor in a patient (e.g., a human patient), the method comprising administering to a patient in need thereof a prophylactically effective regimen or a therapeutically effective regimen, the regimen comprising administering to the patient a compound or composition of the invention, wherein the compound or composition of the invention is administered at a dose that is lower than the human equivalent dosage (HED) of the no observed adverse effect level (NOAEL) over a period of three months, four months, six months, nine months, 1 year, 2 years, 3 years, 4 years or more. The NOAEL, as determined in animal studies, is useful in determining the maximum recommended starting dose for human clinical trials. For instance, the NOAELs can be extrapolated to determine human equivalent dosages. Typically, such extrapolations between species are conducted based on the doses that are normalized to body surface area (i.e., mg/m²). In specific embodiments, the NOAELs are determined in mice, hamsters, rats, ferrets, guinea pigs, rabbits, dogs, primates, primates (monkeys, marmosets, squirrel monkeys, baboons), micropigs or minipigs. For a discussion on the use of NOAELs and their extrapolation to determine human equivalent doses, see Guidance for Industry Estimating the Maximum Safe Starting Dose in Initial Clinical Trials for Therapeutics in Adult Healthy Volunteers, U.S. Department of Health and Human Services Food and Drug Administration Center for Drug Evaluation and Research (CDER), Pharmacology and Toxicology, July 2005.

While not being bound by any specific theory, by the administration of the prophylactically and/or therapeutically effective regimens, the amount of cancer stem cells in a tumor is stabilized or reduced, so as to limit or prevent the potential repopulation of the tumor. In some embodiments since the prophylactically and/or therapeutically effective regimens typically comprise administration of relatively low doses of the compounds, the potential for toxic side effects is likely diminished.

In an additional embodiment, another aspect of the invention is that the administration of the prophylactically and/or therapeutically effective regimen occurs for longer periods of time and/or more frequently relative and/or at lower doses to what is known or used by one skilled in the art. The dosage regimens are further described in section 5.3.1.

In certain embodiments of these aspects, the regimens comprise administering a prophylactically effective regimen and/or a therapeutically effective regimen, wherein the regimen results in a reduction in the amount of cancer stem cells in the patient. In one embodiment, the patient undergoing the regimen is monitored to determine whether the regimen has resulted in a reduction in the amount of cancer stem cells.

Typically, the monitoring of cancer stem cells is conducted by detecting the amount of cancer stem cells in a specimen extracted from the patient. Methods of detecting the amount of cancer stem cells in a specimen are described infra in Section 5.4. This monitoring step is typically performed at least 1, 2, 4, 6, 8, 10, 12, 14, 15, 16, 18, 20, 30, 45, 60, 90, 120, 240 days after the patient begins receiving the regimen.

In some embodiments, the specimen may be a blood or bone marrow specimen, wherein the amount of cancer stem cells per unit of volume (e.g., 1 mL) or other measured unit (e.g., per unit field in the case of a histological analysis) is quantitated. In certain embodiments, the amount of cancer stem cells is determined as a portion (e.g., a percentage) of the cancer cells present in the blood or bone marrow specimen, as a subset of the cancer cells present in the blood or bone marrow specimen, or as a subset of a subset of the cancer cells present in the blood or bone marrow specimen. The amount of cancer stem cells, in other embodiments, can be determined as a percentage of the total blood cells.

In other embodiments, the specimen extracted from the patient is a tissue specimen (e.g., a biopsy extracted from suspected cancerous tissue), where the amount of cancer stem cells can be measured, for example, on the basis of the amount of cancer stem cells per unit weight of the tissue. In certain embodiments, the cancer stem cell population is determined as a portion (e.g., a percentage) of the cancer cells present in the tissue, as a subset of the cancer cells present in the tissue, or as a subset of a subset of the cancer cells present in the tissue.

The amount of cancer stem cells in the extracted specimen can be compared with the amounts of cancer stem cells measured in reference samples to assess the efficacy of the regimen, and the amelioration of the cancer under therapy. In one embodiment, the reference sample is a specimen extracted from the patient undergoing therapy, wherein the specimen is extracted from the patient at an earlier time point (e.g., prior to receiving the regimen, as a baseline reference sample, or at an earlier time point while receiving the therapy). In another embodiment, the reference sample is extracted from a healthy, noncancer-afflicted patient.

In other embodiments the amount of cancer stem cells in the extracted specimen can be compared with a predetermined reference range. In a specific embodiment, the predetermined reference range is based on the amount of cancer stem cells obtained from a population(s) of patients suffering from the same type of cancer as the patient undergoing the therapy.

If the reduction in the amount of cancer stem cells is determined to be too small upon comparing the cancer stem cell population in the specimen extracted from the patient undergoing the regimen with the reference specimen, then the medical practitioner has a number of options to adjust the regimen. For instance, the medical practitioner can then increase either the dosage of the compound or composition of the invention administered, the frequency of the administration, the duration of administration, or any combination thereof. In a specific embodiment, after the determination is made, an additional effective amount of a compound or composition of the invention can be administered to the patient.

In other embodiments, the regimens comprise administering a prophylactically effective regimen and/or a therapeutically effective regimen, wherein the regimen results in a reduction in the cancer cell population in the patient. In one embodiment, the patient undergoing the regimen is monitored to determine whether the regimen has resulted in a reduction in the cancer cell population in the patient.

Typically, the monitoring of the cancer cell population is conducted by detecting the amount of cancer cells in a specimen extracted from the patient. Methods of detecting the amount of cancer cells in a specimen are described infra in Section 5.5. This monitoring step is typically performed at least 1, 2, 4, 6, 8, 10, 12, 14, 15, 16, 18, 20, 30, 45, 60, 90, 120, 240 days after the patient begins receiving the regimen.

In some embodiments, the specimen may be a blood or bone marrow specimen, wherein the amount of cancer cells per unit of volume (e.g., 1 mL) or other measured unit (e.g., per unit field in the case of a histological analysis) is quantitated. The cancer cell population, in certain embodiments, can be determined as a percentage of the total blood cells.

In other embodiments, the specimen extracted from the patient is a tissue specimen (e.g., a biopsy extracted from suspected cancerous tissue), where the amount of cancer cells can be measured, for example, on the basis of the amount of cancer cells per unit weight of the tissue.

The amount of cancer cells in the extracted specimen can be compared with the amount of cancer cells measured in reference samples to assess the efficacy of the regimen and amelioration of the cancer under therapy. In one embodiment, the reference sample is a specimen extracted from the patient undergoing therapy, wherein the specimen from the patient is extracted at an earlier time point (e.g., prior to receiving the regimen, as a baseline reference sample, or at an earlier time point while receiving the therapy). In another embodiment, the reference sample is extracted from a healthy, noncancer-afflicted patient.

In other embodiments the cancer cell population in the extracted specimen can be compared with a predetermined reference range. In a specific embodiment, the predetermined reference range is based on the amount of cancer cells obtained from a population(s) of patients suffering from the same type of cancer as the patient undergoing the therapy.

If the reduction in the cancer cell population is judged too small upon comparing the amount of cancer cells in the specimen extracted from the patients undergoing therapy with the reference specimen, then the medical practitioner has a number of options to adjust the therapeutic regimen. For instance, the medical practitioner can then either increase the dosage of the compound or composition of the invention administered, the frequency of the administration, the duration of administration, or any combination thereof. In a specific embodiment, after the determination is made, a second effective amount of a compound or composition of the invention can be administered to the patient.

In other embodiments, the regimens comprise administering a compound or composition of the invention, wherein the regimen results in a reduction in the amount of cancer stem cells and a reduction in the amount of cancer cells in the patient.

The present invention is also directed to a method for purging bone marrow or peripheral blood prior to autologous stem cell transplant, comprising contacting ex vivo bone marrow or peripheral blood obtained from a human with a composition comprising an amount of a compound of the invention for a time sufficient to significantly purge the cancer stem cells from the bone marrow or peripheral. In an aspect of this embodiment, the amount of cancer stem cells after contacting with a compound of the invention can be decreased by at least 50%, 60%, 75%, 80%, 90%, 95%, or by at least 99%. The present invention is also directed to a method for performing an autologous bone marrow or peripheral blood stem cell transplant, comprising administering to a human an amount of significantly purged bone marrow or peripheral blood effective to reconstitute hematopoietic function in said human, wherein said purged bone marrow or peripheral blood is bone marrow or peripheral blood obtained from said human previously contacted with an amount of a compound of the invention for a time sufficient to significantly purge the bone marrow or peripheral blood of cancer stem cells. Further, the present invention is directed to a composition comprising purged bone marrow or peripheral blood, wherein said purged bone marrow or peripheral blood is bone marrow or peripheral blood obtained from a human and contacted ex vivo with an amount of a compound of the invention for a time sufficient to significantly purge the bone marrow or peripheral blood of cancer stem cells. In one aspect, the composition can further comprise a pharmaceutically acceptable carrier.

5.3.1 Dosage Regimens

The amount of a compound or pharmaceutical composition of the invention used in the prophylactic and/or therapeutic regimens which will be effective in the prevention, treatment, and/or management of cancer can be determined by methods disclosed herein. The frequency and dosage will vary also according to factors specific for each patient depending on the specific compounds administered, the severity of the cancerous condition, the route of administration, as well as age, body, weight, response, and the past medical history of the patient. For example, the dosage of a compound of the invention which will be effective in the treatment, prevention, and/or management of cancer can be determined by administering the compound to an animal model such as, e.g., the animal models disclosed herein or known in to those skilled in the art. See Section 5.7.2, infra. In addition, in vitro assays may optionally be employed to help identify optimal dosage ranges. See Section 5.7.1, infra.

In some embodiments, the prophylactic and/or therapeutic regimens comprise titrating the dosages administered to the patient so as to achieve a specified measure of therapeutic efficacy. Such measures include a reduction in the amount of cancer stem cells in the patient and/or a reduction in the cancer cells in the patient.

In some embodiments, the prophylactic and/or therapeutic regimens comprise administering dosages of a compound or pharmaceutical composition of the invention that are effective to cause a reduction of cancer stem cells. Methods that can be used to determine the reduction of the cancer stem cells in a patient undergoing therapy are discussed infra in Section 5.4.

In certain embodiments, the dosage of the compound of the invention in the prophylactic and/or therapeutic regimen is adjusted so as to achieve a reduction in the amount of cancer stem cells found in a test specimen extracted from a patient after undergoing the therapeutic regimen, as compared with a reference sample. Here, the specimen is a sample extracted from the patient undergoing therapy, wherein the reference sample is extracted from the patient at an earlier time point. In one embodiment, the reference sample is a specimen extracted from the same patient, prior to receiving the prophylactic or therapeutic regimen. In specific embodiments, the amount of cancer stem cells in the test specimen is at least 1%, 2%, 5%, 10%, 15%, 20%, 30%, 40%, 50%, 60%, 70%, 80%, 90%, 95% or 99% lower than in the reference sample.

In other embodiments, the dosage of the compound of the invention in the prophylactic and/or therapeutic regimen is adjusted so as to achieve a reduction in the amount of cancer stem cells found in a test specimen extracted from a patient after undergoing the prophylactic and/or therapeutic regimen, as compared with a reference sample, wherein the reference sample specimen is extracted from a healthy, noncancer-afflicted patient. In specific embodiments, the amount of cancer stem cells in the test specimen is at least within 60%, 50%, 40%, 30%, 20%, 15%, 10%, 5%, or 1%, 2% of the amount of cancer stem cells in the reference sample.

In some embodiments, the dosage of the compound of the invention in the prophylactic and/or therapeutic regimen is adjusted so as to achieve an amount of cancer stem cells that falls within a predetermined reference range. In these embodiments, the amount of cancer stem cells in a test specimen is compared with a predetermined reference range. In a specific embodiment, the predetermined reference range is based on the amount of cancer stem cells obtained from a population(s) of patients suffering from the same type of cancer as the patient undergoing the therapy.

In certain embodiments, the dosage of the compound of the invention in the prophylactic and/or therapeutic regimen is adjusted depending on the amount of cancer stem cells from a sample specimen in comparison with the amount of cancer stem cells in a specimen taken from the same patient either prior to or at an earlier time point in therapy.

In some embodiments, the prophylactic and/or therapeutic regimens comprise administering dosages of a compound or pharmaceutical composition of the invention that are effective to reduce the cancer cell population. Methods that can be used to determine the cancer cell population in a patient undergoing treatment are discussed infra in Section 5.5.

In certain embodiments, the dosage of the compound of the invention in the prophylactic and/or therapeutic regimen is adjusted so as to achieve a reduction in the amount of cancer cells found in a test specimen extracted from a patient after undergoing the prophylactic and/or therapeutic regimen, as compared with a reference sample. Here, the reference sample is a specimen extracted from the patient undergoing therapy, wherein the specimen is extracted from the patient at an earlier time point. In one embodiment, the reference sample is a specimen extracted from the same patient, prior to receiving the prophylactic and/or therapeutic regimen. In specific embodiments, the amount of cancer cells in the test specimen is at least 1%, 2%, 5%, 10%, 15%, 20%, 30%, 40%, 50%, 60%, 70%, 80%, 90%, 95% or 99% lower than in the reference sample.

In some embodiments, the dosage of the compound of the invention in the prophylactic and/or therapeutic regimen is adjusted so as to achieve a amount of cancer cells that falls within a predetermined reference range. In these embodiments, the amount of cancer cells in a test specimen is compared with a predetermined reference range.

In certain embodiments, the dosage of the compound of the invention in the prophylactic and/or therapeutic regimen is adjusted depending on the amount of cancer cells from a sample specimen in comparison with the amount of cancer cells in a specimen taken from the same patient either prior to or at an earlier time point in therapy.

In other embodiments, the dosage of the compound of the invention in prophylactic and/or therapeutic regimen is adjusted so as to achieve a reduction in the amount of cancer cells found in a test specimen extracted from a patient after undergoing the prophylactic and/or therapeutic regimen, as compared with a reference sample, wherein the reference sample is a specimen is extracted from a healthy, noncancer-afflicted patient. In specific embodiments, the amount of cancer cells in the test specimen is at least within 60%, 50%, 40%, 30%, 20%, 15%, 10%, 5%, or 2% of amount of cancer cells in the reference sample.

In treating certain human patients having solid tumors, extracting multiple tissue specimens from a suspected tumor site may prove impracticable. In these embodiments, the dosage of the compounds of the invention in the prophylactic and/or therapeutic regimen for a human patient is extrapolated from doses in animal models that are effective to reduce the cancer stem cell population in those animal models. In the animal models, the prophylactic and/or therapeutic regimens are adjusted so as to achieve a reduction in the amount of cancer stem cells found in a test specimen extracted from an animal after undergoing the prophylactic and/or therapeutic regimen, as compared with a reference sample. The reference sample can be a specimen extracted from the same animal, prior to receiving the prophylactic and/or therapeutic regimen. In specific embodiments, or the amount of cancer stem cells in the test specimen is at least 2%, 5%, 10%, 15%, 20%, 30%, 40%, 50% or 60% lower than in the reference sample. The doses effective in reducing the amount of cancer stem cells in the animals can be normalized to body surface area (e.g., mg/m²) to provide an equivalent human dose.

The prophylactic and/or therapeutic regimens disclosed herein comprise administration of compounds of the invention or pharmaceutical compositions thereof to the patient in a single dose or in multiple doses (e.g., 1, 2, 3, 4, 5, 6, 7, 8, 10, 15, 20, or more doses).

In one embodiment, the prophylactic and/or therapeutic regimens comprise administration of the compounds of the invention or pharmaceutical compositions thereof in multiple doses. When administered in multiple doses, the compounds or pharmaceutical compositions are administered with a frequency and in an amount sufficient to prevent, treat, and/or manage the condition. In one embodiment, the frequency of administration ranges from once a day up to about once every eight weeks. In another embodiment, the frequency of administration ranges from about once a week up to about once every six weeks. In another embodiment, the frequency of administration ranges from about once every three weeks up to about once every four weeks. In another embodiment, the compounds of the invention are delivered continuously to the patient, e.g. intravenously or using a drug pump.

In some embodiments of the invention, the dosage of a compound of the invention or pharmaceutical composition thereof administered is at least 1.5, 1.6, 1.8, 2, 2.5, 3, 4, 5, 6, 7, 8, 10, 20, 30, 40, 50, 60, 70, 80, 90 or 100 times lower than the maximum tolerated dose (MTD) over a period of three months, four months, six months, nine months, 1 year, 2 years, 3 years, 4 years or more.

In some embodiments of the invention, the dosage of a compound of the invention or pharmaceutical composition thereof administered is at least 1.1, 1.2, 1.3, 1.4, 1.5, 1.6, 1.8, 2, 2.5, 3, 4, 5, 6, 7, 8, 10, 20, 30, 40, 50, 60, 70, 80, 90 or 100 times lower than the human equivalent dose (HED) of the no observed adverse effect level (NOAEL) over a period of three months, four months, six months, nine months, 1 year, 2 years, 3 years, 4 years or more. In specific embodiments, the NOAELs are determined in mice, hamsters, rats, ferrets, guinea pigs, rabbits, dogs, primates, primates (monkeys, marmosets, squirrel monkeys, baboons), micropigs or minipigs. See the discussion regarding the NOAELs and HEDs in Section 5.3, supra.

In some embodiments of the invention, the dosage of a compound of the invention or pharmaceutical composition thereof administered is at least 1.1, 1.2, 1.3, 1.4, 1.5, 1.6, 1.8, 2, 2.5, 3, 4, 5, 6, 7, 8, 10, 20, 30, 40, 50, 60, 70, 80, 90 or 100 times lower than the dosage known or used by one skilled in the art.

Generally, the dosage of a compound of the invention administered to a subject to prevent, treat, and/or manage cancer is in the range of 0.01 to 500 mg/kg, and more typically, in the range of 0.1 mg/kg to 100 mg/kg, of the subject's body weight. In one embodiment, the dosage administered to a subject is in the range of 0.1 mg/kg to 50 mg/kg, or 1 mg/kg to 50 mg/kg, of the subject's body weight, more preferably in the range of 0.1 mg/kg to 25 mg/kg, or 1 mg/kg to 25 mg/kg, of the patient's body weight.

In a specific embodiment, the dosage of a compound of the invention administered to a subject to prevent, treat, and/or manage cancer in a patient is 500 mg/kg or less, preferably 250 mg/kg or less, 100 mg/kg or less, 95 mg/kg or less, 90 mg/kg or less, 85 mg/kg or less, 80 mg/kg or less, 75 mg/kg or less, 70 mg/kg or less, 65 mg/kg or less, 60 mg/kg or less, 55 mg/kg or less, 50 mg/kg or less, 45 mg/kg or less, 40 mg/kg or less, 35 mg/kg or less, 30 mg/kg or less, 25 mg/kg or less, 20 mg/kg or less, 15 mg/kg or less, 10 mg/kg or less, 5 mg/kg or less, 2.5 mg/kg or less, 2 mg/kg or less, 1.5 mg/kg or less, or 1 mg/kg or less of a patient's body weight.

In another specific embodiment, the dosage of a compound of the invention administered to a subject to prevent, treat, and/or manage cancer in a patient is a unit dose of 0.1 to 50 mg, 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

In a specific embodiment, the dosage of a compound of the invention administered to a subject to prevent, treat, and/or manage cancer in a patient is in the range of 0.01 to 10 g/m², and more typically, in the range of 0.1 g/m² to 7.5 g/m², of the subject's body surface. In one embodiment, the dosage administered to a subject is in the range of 0.5 g/m² to 5 g/m², or 1 g/m² to 5 g/m² of the subject's body's surface area.

In other embodiments, the prophylactic and/or therapeutic regimen comprises administering to a patient one or more doses of an effective amount of a compound of the invention, wherein the dose of an effective amount achieves a plasma level of at least 0.1 μg/mL, at least 0.5 μg/mL, at least 1 μg/mL, at least 2 μg/mL, at least 5 μg/mL, at least 6 μg/mL, at least 10 μg/mL, at least 15 μg/mL, at least 20 μg/mL, at least 25 μg/mL, at least 50 μg/mL, at least 100 μg/mL, at least 125 μg/mL, at least 150 μg/mL, at least 175 μg/mL, at least 200 μg/mL, at least 225 μg/mL, at least 250 μg/mL, at least 275 μg/mL, at least 300 μg/mL, at least 325 μg/mL, at least 350 μg/mL, at least 375 μg/mL, or at least 400 μg/mL of the compound of the invention.

In other embodiments, the prophylactic and/or therapeutic regimen comprises administering to a patient a plurality of doses of an effective amount of a compound of the invention, wherein the plurality of doses maintains a plasma level of at least 0.1 μg/mL, at least 0.5 μg/mL, at least 1 μg/mL, at least 2 μg/mL, at least 5 μg/mL, at least 6 μg/mL, at least 10 μg/mL, at least 15 μg/mL, at least 20 μg/mL, at least 25 μg/mL, at least 50 μg/mL, at least 100 μg/mL, at least 125 μg/mL, at least 150 μg/mL, at least 175 μg/mL, at least 200 μg/mL, at least 225 μg/mL, at least 250 μg/mL, at least 275 μg/mL, at least 300 μg/mL, at least 325 μg/mL, at least 350 μg/mL, at least 375 μg/mL, or at least 400 μg/mL of the compound of the invention for at least 1 month, 2 months, 3 months, 4 months, 5 months, 6 months, 7 months, 8 months, 9 months, 10 months, 11 months, 12 months, 15 months, 18 months, or 24 months.

In certain embodiments of the invention, the prophylactic and/or therapeutic regimen comprises administration of cantharidin, norcantharidin or sodium cantharidate. In specific embodiments, the prophylactic and/or therapeutic regimen comprises administering to a patient a unit dose of 0.1 to 50 mg, 0.1 to 25 mg, or 0.1 to 20 mg of cantharidin, norcantharidin or sodium cantharidate. In specific embodiments, the prophylactic and/or therapeutic regimen comprises administering to a patient a daily dosage of 0.1 to 50 mg/day, 0.1 to 25 mg/day, or 0.1 to 20 mg/day of cantharidin, norcantharidin or sodium cantharidate.

In some embodiments, the prophylactic and/or therapeutic regimen comprises administration of a compound of the invention in combination with one or more additional anticancer therapeutics. See Section 5.3.2. Preferably, the dosages of the one or more additional anticancer therapeutics used in the combination therapy is lower than those which have been or are currently being used to prevent, treat, and/or manage cancer. The recommended dosages of the one or more additional anticancer therapeutics currently used for the prevention, treatment, and/or management of cancer can be obtained from any reference in the art including, but not limited to, Hardman et al., eds., Goodman & Gilman's The Pharmacological Basis Of Basis Of Therapeutics, 10th ed., Mc-Graw-Hill, New York, 2001; Physician's Desk Reference (60^{th} ed., 2006), which is incorporated herein by reference in its entirety.

In some embodiments, the prophylactic and/or therapeutic regimen comprises administration of a compound of the invention as single active agent or in combination at lower dosage known or used by one skilled in the art.

In certain embodiments the compounds of the invention are administered at lower dose for a longer time to target cancer stem cells. Specific dosage regimens are set forth in this section.

The compound of the invention and the one or more additional anticancer therapeutics can be administered separately, simultaneously, or sequentially. In various embodiments, the compound of the invention and the additional anticancer therapeutic are administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In preferred embodiments, two or more anticancer therapeutics are administered within the same patient visit.

In certain embodiments, the compound of the invention and the additional anticancer therapeutic are cyclically administered. Cycling therapy involves the administration of one anticancer therapeutic for a period of time, followed by the administration of a second anticancer therapeutic for a period of time and repeating this sequential administration, i.e., the cycle, in order to reduce the development of resistance to one or both of the anticancer therapeutics, to avoid or reduce the side effects of one or both of the anticancer therapeutics, and/or to improve the efficacy of the therapies.

In a preferred embodiment, the anticancer therapeutics are administered concurrently to a subject in separate compositions. The combination anticancer therapeutics of the invention may be administered to a subject by the same or different routes of administration.

In a specific embodiment, cycling therapy involves the administration of a first anticancer therapeutic for a period of time, followed by the administration of a second anticancer therapeutic for a period of time, optionally, followed by the administration of a third anticancer therapeutic for a period of time and so forth, and repeating this sequential administration, i.e., the cycle in order to reduce the development of resistance to one of the anticancer therapeutics, to avoid or reduce the side effects of one of the anticancer therapeutics, and/or to improve the efficacy of the anticancer therapeutics.

When a compound of the invention and the additional anticancer therapeutic are administered to a subject concurrently, the term “concurrently” is not limited to the administration of the anticancer therapeutics at exactly the same time, but rather, it is meant that they are administered to a subject in a sequence and within a time interval such that they can act together (e.g., synergistically to provide an increased benefit than if they were administered otherwise). For example, the anticancer therapeutics may be administered at the same time or sequentially in any order at different points in time; however, if not administered at the same time, they should be administered sufficiently close in time so as to provide the desired therapeutic effect, preferably in a synergistic fashion. The combination anticancer therapeutics of the invention can be administered separately, in any appropriate form and by any suitable route. When the components of the combination anticancer therapeutics are not administered in the same pharmaceutical composition, it is understood that they can be administered in any order to a subject in need thereof. For example, a compound of the invention can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, I week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the additional anticancer therapeutic, to a subject in need thereof. In various embodiments, the anticancer therapeutics are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one embodiment, the anticancer therapeutics are administered within the same office visit. In another embodiment, the combination anticancer therapeutics of the invention are administered at 1 minute to 24 hours apart.

5.3.2 Other Therapies

The present invention also provides methods for preventing, treating, and/or managing cancer, the methods comprising administering to a patient (e.g., a human patient) in need thereof, a prophylactically or a therapeutically effective regimen, the regimen comprising administering to the patient a compound of the invention and one or more additional therapies, said additional therapy not being compounds of the invention. The compound of the invention and the additional therapy can be administered separately, concurrently, or sequentially. The combination of agents can act additively or synergistically.

Any therapy (e.g., therapeutic or prophylactic agent) which is useful, has been used, or is currently being used for the prevention, treatment, and/or management of cancer can be used in compositions and method of the invention. Therapies (e.g., therapeutic or prophylactic agents) include, but are not limited to, peptides, antibodies, polypeptides, fusion proteins, nucleic acid molecules, small molecules, mimetic agents, synthetic drugs, inorganic molecules, vaccines, antibodies and organic molecules. Non-limiting examples of cancer therapies include chemotherapies, radiation therapies, hormonal therapies, small molecule therapies, toxin therapies, demethylation therapies, histone deacetylase inhibitor therapies, targeted therapies, epigenetic therapies, differentiation therapies, antiangiogenic therapies, biologic therapies, immunotherapies, or surgery. In certain embodiments, a prophylactically and/or therapeutically effective regimen of the invention comprises the administration of a combination of therapies.

Any therapy (e.g. therapeutic or prophylactic agent) which is acting on a target or is a compound belonging to one of the classes named below in this paragraph may be used in compositions and methods of the invention. Non limiting examples of agents, such as those that target or affect cancer stem cells, include: inhibitors of interleukin-3 receptor (IL-3R) and CD123 (including peptides, peptide-conjugates, antibodies, antibody-conjugates, antibody fragments, and antibody fragment-conjugates that target IL-3R or CD123), cantharidin, norcantharidin and analogs and derivatives thereof, Notch pathway inhibitors including gamma secretase inhibitors, sonic hedgehog/smoothened pathway inhibitors including cyclopamine and analogs thereof, antibodies to CD96, certain NF-kB/proteasome inhibitors including parthenolide and analogs thereof, certain triterpenes including celastrol, certain mTOR inhibitors, compounds and antibodies that target the urokinase receptor, sinefungin, certain inosine monophosphate dehydrogenase (IMPDH) inhibitors, PPAR-alpha and PPAR-gamma agonists and antagonists (including pioglitazone, tesaslitazar, muraglitazar, peliglitazar, lobeglitazone, balaglitazone, ragaglitazar, rosiglitazone, farglitazar, sodelglitazar, reglitazar, naveglitazar, oxeglitazar, metaglidasen, netoglitazone, darglitazone, englitazone, thiazolidinediones, aleglitazar, edaglitazone, rivoglitazone, troglitazone, imiglitazar, and sipoglitazar) telomerase inhibitors, antibodies to EpCAM (ESA), GSK-3 beta agonists and antagonists (including Lithium, 6-bromoinirubin-3′-oxime (BIO), TDZD8), Wnt pathway inhibitors including antibodies to frizzled or small molecules that inhibit disheveled/frizzled or beta catenin, anti-CD20 antibodies and conjugates (e.g. Rituxan, Bexxar, Zevalin) for novel use in multiple myeloma or melanoma, anti-CD133 antibody, anti-CD44 antibody, antibodies to IL-4, certain differentiation agents such as versnarinone, compounds that target CD33 such as an antibody or betulinic acid, compounds that target lactadherin such as an antibody, small molecules or antibodies that target CXCR4 or SDF-1, small molecules or antibodies that target multi-drug resistance pumps, inhibitors of survivin, inhibitors of XIAP, small molecules that target Bcl-2, antibodies to CLL-1, furin inhibitors (such as cucurbitacins).

An additional non-limiting list of compounds that could also be used to target cancer stem cells includes i) antibodies, antibody fragments, and proteins that are either naked or conjugated to a therapeutic moiety that target certain cell surface targets on cancer stem cells, or ii) small molecules known in the art including ones that can be further optimized (e.g., via chemistry) or identified via a cancer stem cell-based screen (e.g. such as one that would determine whether a compound impairs proliferation or viability of a cancer stem cell through standard methods, the cell surface and intracellular targets including (not meant to be exhaustive) are: Rex1 (Zfp42), CTGF, Activin A, Wnt, FGF-2, HIF-1, AP-2gamma, Bmi-1, nucleostemin, hiwi, Moz-TIF2, Nanog, beta-arrestin-2, Oct-4, Sox2, stella, GDF3, RUNX3, EBAF, TDGF-1, nodal, ZFPY, PTNE, Evi-1, Pax3, Mcl-1, c-kit, Lex-1, Zfx, lactadherin, aldehyde dehydrogenase, BCRP, telomerase, CD133, Bcl-2, CD26, Gremlin, and FoxC2.

Examples of cancer therapies include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthracyclin; anthramycin; asparaginase; asperlin; azacitidine (Vidaza); azetepa; azotomycin; batimastat; benzodepa; bicalutamide; bisantrene hydrochloride; bisnafide dimesylate; bisphosphonates (e.g., pamidronate (Aredria), sodium clondronate (Bonefos), zoledronic acid (Zometa), alendronate (Fosamax), etidronate, ibandornate, cimadronate, risedromate, and tiludromate); bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine (Ara-C); dacarbazine; dactinomycin; daunorubicin hydrochloride; decitabine (Dacogen); demethylation agents; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; EphA2 inhibitors; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; herceptin; histone deacetylase inhibitors (HDACs); hydroxyurea; idarubicin hydrochloride; ifosfamide; ilmofosine; imatinib mesylate (Gleevec, Glivec); interleukin II (including recombinant interleukin II, or rIL2), interferon alpha-2a; interferon alpha-2b; interferon alpha-nl; interferon alpha-n3; interferon beta-I a; interferon gamma-I b; iproplatin; irinotecan hydrochloride; lanreotide acetate; lenalidomide (Revlimid); letrozole; leuprolide acetate; liarozole hydrochloride; lometrexol sodium; lomustine; losoxantrone hydrochloride; masoprocol; maytansine; mechlorethamine hydrochloride; anti-CD2 antibodies (e.g., siplizumab (MedImmune Inc.; International Publication No. WO 02/098370, which is incorporated herein by reference in its entirety)); megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxaliplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other examples of cancer therapies include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; HMG CoA reductase inhibitors (e.g., atorvastatin, cerivastatin, fluvastatin, lescol, lupitor, lovastatin, rosuvastatin, and simvastatin); hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; LFA-3TIP (Biogen, Cambridge, Mass.; International Publication No. WO 93/0686 and U.S. Pat. No. 6,162,432); liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene prophylactically and/or therapeutically effective regimens; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone BI; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; gamma secretase inhibitors, single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; 5-fluorouracil; leucovorin; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; thalidomide; velaresol; veramine; verdins; verteporfin; vinorelbine; vinxaltine; anti-integrin antibodies (e.g., anti-integrin αᵥβ₃ antibodies); vorozole; zanoterone; zeniplatin; zilascorb; and zinostatin stimalamer.

In some embodiments, the therapy(ies) used in combination with a compound of the invention is an immunomodulatory agent. Non-limiting examples of immunomodulatory agents include proteinaceous agents such as cytokines, peptide mimetics, and antibodies (e.g., human, humanized, chimeric, monoclonal, polyclonal, Fvs, ScFvs, Fab or F(ab)₂ fragments or epitope binding fragments), nucleic acid molecules (e.g., antisense nucleic acid molecules and triple helices), small molecules, organic compounds, and inorganic compounds. In particular, immunomodulatory agents include, but are not limited to, methotrexate, leflunomide, cyclophosphamide, cytoxan, Immuran, cyclosporine A, minocycline, azathioprine, antibiotics (e.g., FK506 (tacrolimus)), methylprednisolone (MP), corticosteroids, steroids, mycophenolate mofetil, rapamycin (sirolimus), mizoribine, deoxyspergualin, brequinar, malononitriloamindes (e.g., leflunamide), T cell receptor modulators, cytokine receptor modulators, and modulators mast cell modulators. Other examples of immunomodulatory agents can be found, e.g., in U.S. Publication No. 2005/0002934 A1 at paragraphs 259-275 which is incorporated herein by reference in its entirety. In one embodiment, the immunomodulatory agent is a chemotherapeutic agent. In an alternative embodiment, the immunomodulatory agent is an immunomodulatory agent other than a chemotherapeutic agent. In some embodiments, the therapy(ies) used in accordance with the invention is not an immunomodulatory agent.

In some embodiments, the therapy(ies) used in combination with a compound of the invention is an anti-angiogenic agent. Non-limiting examples of anti-angiogenic agents include proteins, polypeptides, peptides, fusion proteins, antibodies (e.g., human, humanized, chimeric, monoclonal, polyclonal, Fvs, ScFvs, Fab fragments, F(ab)₂ fragments, and antigen-binding fragments thereof) such as antibodies that specifically bind to TNF-α, nucleic acid molecules (e.g., antisense molecules or triple helices), organic molecules, inorganic molecules, and small molecules that reduce or inhibit angiogenesis. Other examples of anti-angiogenic agents can be found, e.g., in U.S. Publication No. 2005/0002934 A1 at paragraphs 277-282, which is incorporated by reference in its entirety. In other embodiments, the therapy(ies) used in accordance with the invention is not an anti-angiogenic agent.

In some embodiments, the therapy(ies) used in combination with a compound of the invention is an inflammatory agent. Non-limiting examples of anti-inflammatory agents include any anti-inflammatory agent, including agents useful in therapies for inflammatory disorders, well-known to one of skill in the art. Non-limiting examples of anti-inflammatory agents include non-steroidal anti-inflammatory drugs (NSAIDs), steroidal anti-inflammatory drugs, anticholinergics (e.g., atropine sulfate, atropine methylnitrate, and ipratropium bromide (ATROVENT™)), beta2-agonists (e.g., abuterol (VENTOLIN™ and PROVENTIL™), bitolterol (TORNALATE™), levalbuterol (XOPONEX™), metaproterenol (ALUPENT™), pirbuterol (MAXAIR™), terbutlaine (BRETHAIRE™ and BRETHINE™), albuterol (PROVENTIL™, REPETABS™, and VOLMAX™), formoterol (FORADIL AEROLIZER™), and salmeterol (SEREVENT™ and SEREVENT DISKUS™)), and methylxanthines (e.g., theophylline (UNIPHYL™, THEO-DUR™, SLO-BID™, AND TEHO-42™)). Examples of NSAIDs include, but are not limited to, aspirin, ibuprofen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™). Such NSAIDs function by inhibiting a cyclooxygenase enzyme (e.g., COX-1 and/or COX-2). Examples of steroidal anti-inflammatory drugs include, but are not limited to, glucocorticoids, dexamethasone (DECADRON™), corticosteroids (e.g., methylprednisolone (MEDROL™)), cortisone, hydrocortisone, prednisone (PREDNISONE™ and DELTASONE™), prednisolone (PRELONE™ and PEDIAPRED™), triamcinolone, azulfidine, and inhibitors of eicosanoids (e.g., prostaglandins, thromboxanes, and leukotrienes. Other examples of anti-inflammatory agents can be found, e.g., in U.S. Publication No. 005/0002934 A1 at paragraphs 290-294, which is incorporated by reference in its entirety. In other embodiments, the therapy(ies) used in accordance with the invention is not an anti-inflammatory agent.

In certain embodiments, the therapy(ies) used is an alkylating agent, a nitrosourea, an antimetabolite, and anthracyclin, a topoisomerase II inhibitor, or a mitotic inhibitor. Alkylating agents include, but are not limited to, busulfan, cisplatin, carboplatin, cholormbucil, cyclophosphamide, ifosfamide, decarbazine, mechlorethamine, mephalen, and themozolomide. Nitrosoureas include, but are not limited to carmustine (BCNU) and lomustine (CCNU). Antimetabolites include but are not limited to 5-fluorouracil, capecitabine, methotrexate, gemcitabine, cytarabine, and fludarabine. Anthracyclins include but are not limited to daunorubicin, doxorubicin, epirubicin, idarubicin, and mitoxantrone. Topoisomerase II inhibitors include, but are not limited to, topotecan, irinotecan, etopiside (VP-16), and teniposide. Mitotic inhibitors include, but are not limited to taxanes (paclitaxel, docetaxel), and the vinca alkaloids (vinblastine, vincristine, and vinorelbine).

In some embodiments, the therapy(ies) used in combination with a compound of the invention is an agent that targets cancer stem cells. In certain embodiments, the agent is a small molecule or a biologic including a peptide- or antibody-based compound. In certain embodiments, the agent is attached directly or indirectly to a therapeutic moiety other than a compound of the invention. A non-limiting list of therapeutic moieties includes those listed above in section 3.1, including, but not limited to, alkylating agents, anti-metabolites, plant alkaloids, cytotoxic agents, chemotherapeutic agents (e.g., a steroid, cytosine arabinoside, fluoruracil, methotrexate, aminopterin, mitomycin C, demecolcine, etoposide, mithramycin, calicheamicin, CC-1065, chlorambucil or melphalan), radionuclides, therapeutic enzymes, cytokines, toxins including plant-derived toxins, fungus-derived toxins, bacteria-derived toxin (e.g., deglycosylated ricin A chain, a ribosome inactivating protein, alpha-sarcin, aspergillin, restirictocin, a ribonuclease, a diphtheria toxin, Pseudomonas exotoxin, a bacterical endotoxin or the lipid A moiety of a bacterial endotoxin), growth modulators and RNase. In some embodiments, the agent used is an agent that binds to a marker, e.g., antigen on cancer stem cells. In a specific embodiment, the agent binds to an antigen that is expressed at a greater level on cancer stem cells than on normal stem cells. In another specific embodiment, the agent binds to an antigen that is expressed at the same level on cancer stem cells as on normal stem cells.

In a specific embodiment, the agent binds to a cancer stem cell antigen. In other embodiments, the therapy(ies) used in accordance with the invention is an agent that binds to a marker on cancer stem cells. Non-limiting examples of antigens on cancer stem cells that can be used to target cancer stem cells such as CD123. Other non limiting examples of cell surface antigens present on a cancer stem cell include CD44, CLL1, CD133, CD34, CD19, CD20, RC2, and α₂β₁. In one embodiment, the agent that binds to a marker on cancer stem cells is a peptide or an antibody that is either naked or conjugated to a therapeutic moiety. In another embodiment, the agent that binds to a marker on cancer stem cells is composed, in whole or in part, of a ligand (e.g., interleukin 3). In certain embodiments, the antibody or ligand is attached directly or indirectly to a therapeutic moiety. Non-limiting examples of therapeutic moieties include, but are not limited to, therapeutic enzymes, chemotherapeutic agents, cytokines, radionuclides, antimetabolites, toxins and RNase.

In certain embodiments, antibodies that bind to a marker on cancer stem cells are substantially non-immunogenic in the treated subject. Strategies to prepare non-immunogenic antibodies include, but are not limited to, chimerizing the antibody, humanizing the antibody, and isolating antibodies from the same species as the subject receiving the therapy. See, for example, paragraphs 539-573 of U.S. Publication No. 2005/0002934 A1, which is incorporated by reference in its entirety. Antibodies that bind to markers on cancer stem cells can be produced using techniques known in the art.

In certain embodiments, antibodies or fragments thereof that bind to a marker on cancer stem cells are substantially non-immunogenic in the treated subject. Methods for obtaining non-immunogenic antibodies include, but are not limited to, chimerizing the antibody, humanizing the antibody, and isolating antibodies from the same species as the subject receiving the therapy. Antibodies or fragments thereof that bind to markers in cancer stem cells can be produced using techniques known in the art. See, for example, paragraphs 539-573 of U.S. Publication No. 2005/0002934 A1, which is incorporated by reference in its entirety.

In some embodiments, a compound of the invention is used in combination with radiation therapy comprising the use of x-rays, gamma rays and other sources of radiation to destroy cancer stem cells and/or cancer cells. In specific embodiments, the radiation therapy is administered as external beam radiation or teletherapy, wherein the radiation is directed from a remote source. In other embodiments, the radiation therapy is administered as internal therapy or brachytherapy wherein a radioactive source is placed inside the body close to cancer stem cells, cancer cells or a tumor mass.

Currently available cancer therapies and their dosages, routes of administration and recommended usage are known in the art and have been described in such literature as the Physician's Desk Reference (60^{th} ed., 2006). In accordance with the present invention, the dosages and frequency of administration of chemotherapeutic agents are described in the Section 5.3.1.

5.3.3 Target Cancers

Any type of cancer can be prevented, treated and/or managed in accordance with the invention. Non-limiting examples of cancers that can be prevented, treated and/or managed in accordance with the invention cancers include: leukemias, such as but not limited to, acute leukemia, acute lymphocytic leukemia, acute myelocytic leukemias, such as, myeloblastic, promyelocytic, myelomonocytic, monocytic, and erythroleukemia leukemias and myelodysplastic syndrome; chronic leukemias, such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to ductal carcinoma, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to papillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, prostatic intraepithelial neoplasia, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypernephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

The prophylactically and/or therapeutically effective regimens are also useful in the treatment, prevention and/or management of a variety of cancers or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T cell lymphoma, Burkitt's lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscarama, and osteosarcoma; and other tumors, including melanoma, xeroderma pigmentosum, keratoactanthoma, seminoma, thyroid follicular cancer and teratocarcinoma. In some embodiments, cancers associated with aberrations in apoptosis are prevented, treated and/or managed in accordance with the methods of the invention. Such cancers may include, but are not limited to, follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders of the skin, lung, liver, bone, brain, stomach, colon, breast, prostate, bladder, kidney, pancreas, ovary, and/or uterus are prevented, treated and/or managed in accordance with the methods of the invention. In other specific embodiments, a sarcoma, or melanoma is prevented, treated and/or managed in accordance with the methods of the invention.

In a specific embodiment, the cancer being prevented, treated, and/or managed in accordance with the invention is leukemia, lymphoma, myeloma or myelodysplastic syndrome.

Non-limiting examples of leukemias and other blood-borne cancers that can be prevented, treated, and/or managed with the methods of the invention include acute lymphoblastic leukemia “ALL”, acute lymphoblastic B-cell leukemia, acute lymphoblastic T-cell leukemia, acute myeloblastic leukemia “AML”, acute promyelocytic leukemia “APL”, acute monoblastic leukemia, acute erythroleukemic leukemia, acute megakaryoblastic leukemia, acute myelomonocytic leukemia, acute nonlymphocyctic leukemia, acute undifferentiated leukemia, myelodysplastic syndrome (“MDS”), chronic myelocytic leukemia “CML”, chronic lymphocytic leukemia “CLL”, and hairy cell leukemia.

Non-limiting examples of lymphomas that can be prevented, treated, and/or managed in accordance with the methods of the invention include Hodgkin's disease, non-Hodgkin's Lymphoma, Multiple myeloma, Waldenstrom's macroglobulinemia, Heavy chain disease, and Polycythemia vera.

In another embodiment, the cancer being prevented, treated, and/or managed in accordance with the invention is a solid tumor. Examples of solid tumors that can be prevented, treated, and/or managed in accordance with the methods of the invention include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiform, astrocytoma, medulloblastoma, craniopharyngioma, ependymoma, pinealoma, hemangioblastoma, acoustic neuroma, oligodendroglioma, meningioma, skin cancer, melanoma, neuroblastoma, and retinoblastoma.

5.3.4 Patient Population

In accordance with the invention, a prophylactically and/or therapeutically effective regimen of the invention is administered to subjects with or expected to develop cancer (e.g., subjects with a genetic predisposition for a particular type of cancer, subject that have been exposed to a carcinogen, or subjects that are in remission from a particular cancer). In a specific embodiment, the subject has been diagnosed with cancer using techniques known to one of skill in the art including, but not limited to, physical examination (e.g., prostate examination, breast examination, lymph nodes examination, abdominal examination, skin surveillance), visual methods (e.g., colonoscopy, bronchoscopy, endoscopy), PAP smear analyses (cervical cancer), stool guaiac analyses, blood tests (e.g., complete blood count (CBC) test, prostate specific antigen (PSA) test, carcinoembryonic antigen (CEA) test, cancer antigen (CA)-125 test, alpha-fetoprotein (AFP)), karyotyping analyses, bone marrow analyses (e.g., in cases of hematological malignancies), histology, cytology, a sputum analysis and imaging methods (e.g., computed tomography (CT), magnetic resonance imaging (MRI), ultrasound, X-ray imaging, mammography, bone scans). Subjects may or may not have been previously treated for cancer.

The prophylactically and/or therapeutically regimens may be used as any line of cancer therapy, e.g., a first line, second line or third line of cancer therapy. In a specific embodiment, the subject to receive or receiving a compound of the invention is receiving or has received other cancer therapies. In another embodiment the subject to receive a compound of the invention is receiving other cancer therapies before any adverse effects or intolerance of these other cancer therapies occurs. In an alternative embodiment, the subject to receive or receiving a compound, of the invention has not received or is not receiving other cancer therapies.

In one embodiment, a compound of the invention is administered to a subject that is undergoing or has undergone surgery to remove a tumor or neoplasm. In a specific embodiment, a compound of the invention is administered to a subject concurrently or following surgery to remove a tumor or neoplasm. In another embodiment, a compound of the invention is administered to a subject before surgery to remove a tumor or neoplasm and, in some embodiments, during and/or after surgery.

In one embodiment, a compound of the invention is administered to a subject after a course of therapy with the goal of killing cancer cells. In some embodiments, the course of therapy involves the administration of bolus doses of chemotherapeutic agents and/or bolus doses of radiation therapy. In a specific embodiment, a compound of the invention is administered to a subject after the subject has received a course of therapy involving maximum tolerated doses or no observed adverse effect level doses of one or more chemotherapeutic agents and/or radiation therapy.

In certain embodiments, a compound of the invention is administered to a subject as an alternative to chemotherapy, radioimmunotherapy, antibody therapy, pro-drug activating enzyme therapy, toxin therapy, protein therapy, radiation therapy, small molecule therapy, hormonal therapy, targeted therapy, epigenetic therapy, demethylation therapy, histone deacetylase inhibitor therapy, differentiation therapy, antiangiogenic therapy and/or biologic therapy, including immunotherapy where the therapy has proven or may prove too toxic, i.e., results in unacceptable or unbearable side effects, for the subject. In some embodiments, a compound of the invention is administered to a subject that is susceptible to adverse reactions from other cancer therapies. The subject may e.g., have a suppressed immune system (e.g., post-operative patients, chemotherapy patients, and patients with immunodeficiency disease), have an impaired renal or liver function, be elderly, be a child, be an infant, have a neuropsychiatric disorder, take a psychotropic drug, have a history of seizures, or be on medication that would negatively interact with the cancer therapies.

In a specific embodiment, a compound of the invention is administered to subjects that will, are or have undergone radiation therapy. Among these subjects are those that have received chemotherapy, hormonal therapy and/or biological therapy including immunotherapy as well as those who have undergone surgery.

In another embodiment, a compound of the invention is administered to subjects that will, are or have received hormonal therapy and/or biological therapy including immunotherapy. Among these subjects are those that have received chemotherapy and/or radiation therapy as well as those who have undergone surgery.

In certain embodiments, a compound of the invention is administered to a subject refractory to one or, more therapies. In one embodiment, that a cancer is refractory to a therapy means that at least some significant portion of the cancer cells are not killed or their cell division arrested. The determination of whether the cancer cells are refractory can be made either in vivo or in vitro by any method known in the art for assaying the effect of a therapy on cancer cells, using the art-accepted meanings of “refractory” in such a context. See, e.g., Section 5.5 for non-limiting examples of methods for determining the effect of a therapy on cancer cells. In various embodiments, a cancer is refractory where the amount of cancer cells has not been significantly reduced, or has increased. In other embodiments, that a cancer is refractory means that cancer stem cells are adequately stabilized, reduced. or eradicated. The determination of whether the cancer stem cells are refractory can be made either in vivo or in vitro by any methods known in the art or described herein. See, e.g., Section 5.4 for non-limiting examples methods for determining the effectiveness of a therapy on cancer stem cells.

In some embodiments, a compound of the invention is administered to reverse resistance or to increase sensitivity of cancer cells to certain hormonal, radiation and chemotherapeutic agents thereby sensitizing the cancer cells to one or more of these agents, which can then be administered (or continue to be administered) to treat or manage cancer, including to prevent metastasis. In a specific embodiment, the compound of the invention is administered to patients with increased levels of the cytokine IL-6, which has been associated with the development of cancer cell resistance to different treatment regimens, such as chemotherapy and hormonal therapy.

In some embodiments, a compound of the invention is administered to a subject with a mean absolute lymphocyte count of at least approximately 400 cells/mm³, at 500 cells/mm³, at least approximately 600 cells/mm³, at least approximately 700 cells/mm³, at least approximately 800 cells/mm³, at least approximately 900 cells/mm³, at least approximately 1000 cells/mm³, at least approximately 1100 cells/mm³, at least approximately 1200 cells/mm³. In other embodiments, a prophylactically and/or therapeutically effective regimen of the invention is administered to a subject with a mean absolute lymphocyte count of approximately 400 cells/mm³ to approximately 1200 cells/mm³, approximately 500 cells/mm³ to approximately 1200 cells/mm³, approximately 600 cells/mm³ to approximately 1200 cells/mm³, approximately 700 cells/mm³ to approximately 1200 cells/mm³, approximately 800 cells/mm³ to approximately 1200 cells/mm³, approximately 900 cells/mm³ to approximately 1200 cells/mm³, approximately 1000 cells/mm³ to approximately 12000 cells/mm³ In a more specific embodiment, the regimen results in a mean absolute lymphocyte count of at least approximately 400 cells/mm³. The mean absolute lymphocyte count can be determined by methods set forth in Section 5.6, infra. In some embodiments, the regimen comprises monitoring the mean absolute lymphocyte count in the human subject.

In some embodiments, a regimen of the invention is administered to a subject with a mean absolute neutrophil count of at least approximately 1000 cells/mm³, at least approximately 1200 cells/mm³, at least approximately 1500 cells/mm³, or at least approximately 2000 cells/mm³. In another embodiments, a regimen of the invention is administered to a subject with a mean absolute neutrophil count of approximately 1000 cells/mm³ to approximately 2500 cells/mm³. In a specific embodiment, the mean absolute neutrophil count is determined by the methods described in Section 5.6, infra. In some embodiments, the regimen comprises monitoring the absolute neutrophil count.

In some embodiments, a compound of the invention is administered to a subject that is in remission. In a specific embodiment, the subject is cancer-free, i.e., no cancer is detectable using a method (e.g. CT, MRI) described herein or known to one of skill in the art.

In some embodiments, a compound of the invention is administered to a subject that failed treatment, relapsed or is refractory.

5.4 Methods of Monitoring Cancer Stem Cells

As part of the prophylactically effective and/or therapeutically effective regimens of the invention, the cancer stem cell population can be monitored to assess the efficacy of a therapy as well as to determine prognosis of a subject with cancer or the efficacy of a therapeutically or prophylactically effective regimen. In certain embodiments of the prophylactically effective and/or therapeutically effective therapies or regimens of the invention, the therapies or regimens result in a stabilization or reduction in the cancer stem cell population in the patient. In one embodiment, the subject undergoing the regimen is monitored to assess whether the regimen has resulted in a stabilization or reduction in the cancer stem cell population in the subject.

In some embodiments, the amount of cancer stem cells in a subject is determined using a technique well-known to one of skill in the art or described below in § 5.7.2.

In accordance with the invention, cancer stem cells comprise a unique subpopulation (often 0.1-10% or so) of a tumor that, in contrast to the remaining 90% or so of the tumor (i.e., the tumor bulk), are relatively more tumorigenic and relatively more slow-growing or quiescent. Given that conventional therapies and regimens have, in large part, been designed to attack rapidly proliferating cells (i.e., those cancer cells that comprise the tumor bulk), slower growing cancer stem cells may be relatively more resistant than faster growing tumor bulk to conventional therapies and regimens. This would explain another reason for the failure of standard oncology treatment regimens to ensure long-term benefit in most patients with advanced stage cancers. In a specific embodiment, a cancer stem cell(s) is the founder cell of a tumor (i.e., it is the progenitor of cancer cells). In some embodiments, a cancer stem cell(s) has one, two, three, or more or all of the following characteristics or properties: (i) can harbor the ability to initiate a tumor and/or to perpetuate tumor growth, (ii) can be generally relatively less mutated than the bulk of a tumor (e.g. due to slower growth and thus fewer DNA replication-dependent errors, improved DNA repair, and/or epigenetic/non-mutagenic changes contributing to their malignancy), (iii) can have many features of a normal stem cell(s) (e.g., similar cell surface antigen and/or intracellular expression profile, self-renewal programs, multi-drug resistance, an immature phenotype, etc., characteristic of normal stem cells) and may be derived from a normal stem cell(s), (iv) can be potentially responsive to its microenvironment (e.g., the cancer stem cells may be capable of being induced to differentiate and/or divide asymmetrically), (v) can be the source of metastases, (vi) can be slow-growing or quiescent, (vii) can be symmetrically-dividing, (viii) can be tumorigenic (e.g. as determined by NOD/SCID implantation experiments), (ix) can be relatively resistant to traditional therapies (i.e. chemoresistant), and (x) can comprise a subpopulation of a tumor (e.g. relative to the tumor bulk).

In other embodiments, the amount of cancer stem cells in a sample from a subject is determined/assessed using a technique described herein or well-known to one of skill in the art. Such samples include, but are not limited to, biological samples and samples derived from a biological sample. In certain embodiments, in addition to the biological sample itself or in addition to material derived from the biological sample such as cells, the sample used in the methods of this invention comprises added water, salts, glycerin, glucose, an antimicrobial agent, paraffin, a chemical stabilizing agent, heparin, an anticoagulant, or a buffering agent. In certain embodiments, the biological sample is blood, serum, urine, bone marrow or interstitial fluid. In another embodiment, the sample is a tissue sample. In a particular embodiment, the tissue sample is breast, brain, skin, colon, lung, liver, ovarian, pancreatic, prostate, renal, bone or skin tissue. In a specific embodiment, the tissue sample is a biopsy of normal or tumor tissue. The amount of biological sample taken from the subject will vary according to the type of biological sample and the method of detection to be employed. In a particular embodiment, the biological sample is blood, serum, urine, or bone marrow and the amount of blood, serum, urine, or bone marrow taken from the subject is 0.1 ml, 0.5 ml, 1 ml, 5 ml, 8 ml, 10 ml or more. In another embodiment, the biological sample is a tissue and the amount of tissue taken from the subject is less than 10 milligrams, less than 25 milligrams, less than 50 milligrams, less than 1 gram, less than 5 grams, less than 10 grams, less than 50 grams, or less than 100 grams.

In accordance with the methods of the invention, a sample derived from a biological sample is one in which the biological sample has been subjected to one or more pretreatment steps prior to the detection and/or measurement of the cancer stem cell population in the sample. In certain embodiments, a biological fluid is pretreated by centrifugation, filtration, precipitation, dialysis, or chromatography, or by a combination of such pretreatment steps. In other embodiments, a tissue sample is pretreated by freezing, chemical fixation, paraffin embedding, dehydration, permeabilization, or homogenization followed by centrifugation, filtration, precipitation, dialysis, or chromatography, or by a combination of such pretreatment steps. In certain embodiments, the sample is pretreated by removing cells other than stem cells or cancer stem cells from the sample, or removing debris from the sample prior to the determination of the amount of cancer stem cells in the sample according to the methods of the invention.

The samples for use in the methods of this invention may be taken from any animal subject, preferably mammal, most preferably a human. The subject from which a sample is obtained and utilized in accordance with the methods of this invention includes, without limitation, an asymptomatic subject, a subject manifesting or exhibiting 1, 2, 3, 4 or more symptoms of cancer, a subject clinically diagnosed as having cancer, a subject predisposed to cancer, a subject suspected of having cancer, a subject undergoing therapy for cancer, a subject that has been medically determined to be free of cancer (e.g., following therapy for the cancer), a subject that is managing cancer, or a subject that has not been diagnosed with cancer. In certain embodiments, the term “has no detectable cancer” as used herein, refers to a subject or subjects in which there is no detectable cancer by conventional methods, e.g. MRI. In other embodiments, the term refers to a subject or subjects free from any disorder.

In certain embodiments, the amount of cancer stem cells in a subject or a sample from a subject assessed prior to therapy or regimen (e.g. at baseline) or at least 1, 2, 4, 6, 7, 8, 10, 12, 14, 15, 16, 18, 20, 30, 60, 90 days, 6 months, 9 months, 12 months, >12 months after the subject begins receiving the therapy or regimen. In certain embodiments, the amount of cancer stem cells is assessed after a certain number of doses (e.g., after 2, 5, 10, 20, 30 or more doses of a therapy). In other embodiments, the amount of cancer stem cells is assessed after 1 week, 2 weeks, 1 month, 2 months, 1 year, 2 years, 3 years, 4 years or more after receiving one or more therapies.

In certain embodiments, a positive or negative control sample is a sample that is obtained or derived from a corresponding tissue or biological fluid or tumor as the sample to be analyzed in accordance with the methods of the invention. This sample may come from the same patient or different persons and at the same or different time points.

For clarity of disclosure, and not by way of limitation, the following pertains to analysis of a blood sample from a patient. However, as one skilled in the art will appreciate, the assays and techniques described herein can be applied to other types of patient samples, including a body fluid (e.g. blood, bone marrow, plasma, urine, bile, ascitic fluid), a tissue sample suspected of containing material derived from a cancer (e.g. a biopsy) or homogenate thereof. The amount of sample to be collected will vary with the particular type of sample and method of determining the amount of cancer stem cells used and will be an amount sufficient to detect the cancer stem cells in the sample.

A sample of blood may be obtained from a patient having different developmental or disease stages. Blood may be drawn from a subject from any part of the body (e.g., a finger, a hand, a wrist, an arm, a leg, a foot, an ankle, a stomach, and a neck) using techniques known to one of skill in the art, in particular methods of phlebotomy known in the art. In a specific embodiment, venous blood is obtained from a subject and utilized in accordance with the methods of the invention. In another embodiment, arterial blood is obtained and utilized in accordance with the methods of the invention. The composition of venous blood varies according to the metabolic needs of the area of the body it is servicing. In contrast, the composition of arterial blood is consistent throughout the body. For routine blood tests, venous blood is generally used.

The amount of blood collected will vary depending upon the site of collection, the amount required for a method of the invention, and the comfort of the subject. In some embodiments, any amount of blood is collected that is sufficient to detect the amount of cancer stem cells. In a specific embodiment, I cc or more of blood is collected from a subject.

The amount of cancer stem cells in a sample can be expressed as the percentage of, e.g., overall cells, overall cancer cells or overall stem cells in the sample, or quantitated relative to area (e.g. cells per high power field), or volume (e.g. cells per ml), or architecture (e.g. cells per bone specula in a bone marrow specimen).

In some embodiments, the sample may be a blood sample, bone marrow sample, or a tissue/tumor biopsy sample, wherein the amount of cancer stem cells per unit of volume (e.g., 1 mL) or other measured unit (e.g., per unit field in the case of a histological analysis) is quantitated. In certain embodiments, the cancer stem cell population is determined as a portion (e.g., a percentage) of the cancerous cells present in the blood or bone marrow or tissue/tumor biopsy sample or as a subset of the cancerous cells present in the blood or bone marrow or tissue/tumor biopsy sample. The cancer stem cell population, in other embodiments, can be determined as a portion (e.g., percentage) of the total cells. In yet other embodiments, the cancer stem cell population is determined as a portion (e.g., a percentage) of the total stem cells present in the blood sample.

In other embodiments, the sample from the patient is a tissue sample (e.g., a biopsy from a subject with or suspected of having cancerous tissue), where the amount of cancer stem cells can be measured, for example, by immunohistochemistry or flow cytometry, or on the basis of the amount of cancer stem cells per unit area, volume, or weight of the tissue. In certain embodiments, the cancer stem cell population (the amount of cancer stem cells) is determined as a portion (e.g., a percentage) of the cancerous cells present in the tissue sample or as a subset of the cancerous cells present in the tissue sample. In yet other embodiments, the cancerous stem cell population (the amount of cancer stem cells) is determined as a portion (e.g., a percentage) of the overall cells or stem cell cells in the tissue sample.

The amount of cancer stem cells in a test sample can be compared with the amount of cancer stem cells in reference sample(s) to assess the efficacy of the regimen. In one embodiment, the reference sample is a sample obtained from the subject undergoing therapy at an earlier time point (e.g., prior to receiving the regimen as a baseline reference sample, or at an earlier time point while receiving the therapy). In this embodiment, the therapy desirably results in a decrease in the amount of cancer stem cells in the test sample as compared with the reference sample. In another embodiment, the reference sample is obtained from a healthy, subject who has no detectable cancer, or from a patient that is in remission for the same type of cancer. In this embodiment, the therapy desirably results in the test sample having an equal amount of cancer stem cells, or less than the amount of cancer stem cells than are detected in the reference sample.

In other embodiments, the cancer stem cell population in a test sample can be compared with a predetermined reference range and/or a previously detected amount of cancer stem cells determined for the subject to gauge the subject's response to the regimens described herein. In a specific embodiment, a stabilization or reduction in the amount of cancer stem cells relative to a predetermined reference range and/or earlier (previously detected) cancer stem cell amount determined for the subject indicates an improvement in the subject's prognosis or a positive response to the regimen, whereas an increase relative to the predetermined reference range and/or earlier cancer stem cell amount indicates the same or worse prognosis, and/or a failure to respond to the regimen. The cancer stem cell amount can be used in conjunction with other measures to assess the prognosis of the subject and/or the efficacy of the regimen. In a specific embodiment, the predetermined reference range is based on the amount of cancer stem cells obtained from a patient or population(s) of patients suffering from the same type of cancer as the patient undergoing the therapy.

Generally, since stem cell antigens can be present on both cancer stem cells and normal stem cells, a sample from the cancer-afflicted patient will have a higher stem cell count than a sample from a healthy, subject who has no detectable cancer due to the presence of the cancer stem cells. The therapy will desirably result in a cancer stem cell count for the test sample (e.g., the sample from the patient undergoing therapy) that decreases and becomes increasingly closer to the stem cell count in a reference sample that is sample from a healthy, subject who has no detectable cancer.

If the reduction in the amount of cancer stem cells is determined to be inadequate upon comparing the amount of cancer stem cells in the sample from the subject undergoing the regimen with the reference sample, then the medical practitioner has a number of possible options to adjust the regimen. For instance, the medical practitioner can then increase either the dosage or intensity of the therapy administered, the frequency of the administration, the duration of administration, combine the therapy with another therapy(ies), change the management altogether including halting therapy, or any combination thereof.

In certain embodiments, the dosage, frequency and/or duration of administration of a therapy is modified as a result of the change in the amount of cancer stem cells detected in or from the treated patient. For example, if a subject receiving therapy for leukemia has a cancer stem cell measurement of 2.5% of his tumor prior to therapy and 5% after 6 weeks of therapy, then the therapy or regimen may be altered or stopped because the increase in the percentage of cancer stem cells indicates that the therapy or regimen is not optimal. Alternatively, if another subject with leukemia has a cancer stem cell measurement of 2.5% of his tumor prior to therapy and 1% after 6 weeks of therapy, then the therapy or regimen may be continued because the decrease in the percentage of cancer stem cells indicates that the therapy or regimen is effective.

The amount of cancer stem cells can be monitored/assessed using standard techniques known to one of skill in the art. Cancer stem cells can be monitored by, e.g., obtaining a sample, such as a tissue/tumor sample, blood sample or a bone marrow sample, from a subject and detecting cancer stem cells in the sample. The amount of cancer stem cells in a sample (which may be expressed as percentages of, e.g., overall cells or overall cancer cells) can be assessed by detecting the expression of antigens on cancer stem cells. Techniques known to those skilled in the art can be used for measuring these activities. Antigen expression can be assayed, for example, by immunoassays including, but not limited to, western blots, immunohistochemistry, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, immunofluorescence, protein A immunoassays, flow cytometry, and FACS analysis. In such circumstances, the amount of cancer stem cells in a test sample from a subject may be determined by comparing the results to the amount of stem cells in a reference sample (e.g., a sample from a subject who has no detectable cancer) or to a predetermined reference range, or to the patient him/herself at an earlier time point (e.g. prior to, or during therapy).

In a specific embodiment, the cancer stem cell population in a sample from a patient is determined by flow cytometry. This method exploits the differential expression of certain surface markers on cancer stem cells relative to the bulk of the tumor. Labeled antibodies (e.g., fluorescent antibodies) can be used to react with the cells in the sample, and the cells are subsequently sorted by FACS or flow cytometry methods. In some embodiments, a combination of cell surface markers are utilized in order to determine the amount of cancer stem cells in the sample. For example, both positive and negative cell sorting may be used to assess the amount of cancer stem cells in the sample. Cancer stem cells for specific tumor types can be determined by assessing the expression of markers on cancer stem cells. In certain embodiments, the tumors harbor cancer stem cells and their associated markers as set forth in Table 2 below, which provides a non-limiting list of cancer stem cell phenotypes associated with various types of cancer.

TABLE 2

| Tumor | Cancer Stem Cell Phenotype |
|---|---|
| Leukemia (AML) | CD34+/CD38− |
| Breast | CD44+/CD24− |
| Brain | CD133+ |
| Leukemia (ALL) | CD34+/CD10−/CD19− |
| Ovarian | CD44+/CD24− |
| Multiple Myeloma | CD138−/CD34−/CD19+ |
| Chronic myelogenous leukemia | CD34+/CD38− |
| Melanoma | CD20+ |
| Ependymoma | CD133+/RC2+ |
| Prostate | CD44+/α₂β₁^{hi}/CD133+ |

Additional cancer stem cell markers include, but are not limited to, CD123, CLL-1, combinations of SLAMs (signaling lymphocyte activation molecule family receptors; see Yilmaz et al., “SLAM family markers are conserved among hematopoietic stem cells from old and reconstituted mice and markedly increase their purity,” Hematopoiesis 107: 924-930 (2006)), such as CD150, CD244, and CD48, and those markers disclosed in U.S. Pat. No. 6,004,528 to Bergstein, in pending U.S. patent application Ser. No. 09/468,286, and in U.S. Patent Application Publication Nos. 2006/0083682, 2007/0036800, 2007/0036801, 2007/0036802, 2007/0041984, 2007/0036803, and 2007/0036804, each of which are incorporated herein by reference in their entirety. See, e.g., Table 1 of U.S. Pat. No. 6,004,528 and Tables 1, 2, and 3 of U.S. patent application Ser. No. 09/468,286 and U.S. Patent Application Publication Nos. 2006/0083682, 2007/0036800, 2007/0036801, 2007/0036802, 2007/0041984, 2007/0036803, and 2007/0036804.

In a specific embodiment the cancer stem population in a sample, e.g., a tissue sample, such as a solid tumor biopsy, is determined using immunohistochemistry techniques. This method exploits the differential expression of certain surface markers on cancer stem cells relative to the bulk of the tumor. Labeled antibodies (e.g., fluorescent antibodies) can be used to react with the cells in the sample, and the tissue is subsequently stained. In some embodiments, a combination of certain cell surface markers are utilized in order to determine the amount of cancer stem cells in the sample. Cancer stem cells for specific tumor types can be determined by assessing the expression of certain markers that are specific to cancer stem cells. In certain embodiments, the tumors harbor cancer stem cells and their associated markers as set forth in Table 2 above.

Suitable cancer stem cell antigens may be identified: (i) through publicly available information, such as published and unpublished expression profiles including cell surface antigens of cancer stem cells of a particular tumor type or adult stem cells for a particular tissue type (e.g. Table 2), and/or (ii) by cloning cancer stem cells or adult stem cells of a particular tumor or tissue type, respectively, in order to determine their expression profiles and complement of cell surface antigens. Cloning of normal stem cells is a technique routinely employed in the art (Uchida et al., “Heterogeneity of hematopoeitic stem cells”, Curr. Opin. Immunol, 5:177-184 (1993)). In fact, this same technique is used to identify normal stem cells and cancer stem cells. Moreover, assumption that a proportion of normal stem cell gene products, e.g. cell surface antigens, will also be present on cancer stem cells derived from the same tissue type has proven an effective way to identify cancer stem cell gene products and cancer stem cells. For example, knowledge that the normal hematopoietic stem cell was CD34+/CD38− resulted in the determination that acute myeloid leukemia (AML) stem cells is similarly CD34+/CD38−. This indeed was confirmed by standard stem cell cloning techniques (See Bonnet et al., “Human acute myeloid leukemia is organized as a hierarchy that originates from a primitive hematopoietic cell,” Nat Med 3:730-737 (1997)). Brain cancer stem cells were similarly isolated using a marker of normal (brain) stem cells, in this case CD133 (See Singh et al. Identification of human brain tumor initiating cells. Nature 432(7015):396-401 (2004)).

In certain embodiments using flow cytometry of a sample, the Hoechst dye protocol can be used to identify cancer stem cells in tumors. Briefly, two Hoechst dyes of different colors (typically red and blue) are incubated with tumor cells. The cancer stem cells, in comparison with bulk cancer cells, over-express dye efflux pumps on their surface that allow these cells to pump the dye back out of the cell. Bulk tumor cells largely have fewer of these pumps, and are therefore relatively positive for the dye, which can be detected by flow cytometry. Typically a gradient of dye positive (“dye⁺”) vs. dye negative (“dye-”) cells emerges when the entire population of cells is observed. Cancer stem cells are contained in the dye- or dye low (dyelow) population. For an example of the use of the Hoechst dye protocol to characterize a stem cell or cancer stem cell population see Goodell et al., “A leukemic stem cell with intrinsic drug efflux pump capacity in acute myeloid leukemia,” Blood, 98(4):1166-1173 (2001) and Kondo et al., “Persistence of a small population of cancer stem-like cells in the C6 glioma cell line,” Proc. Natl. Acad. Sci. USA 101:781-786 (2004). In this way, flow cytometry could be used to measure cancer stem cell amount pre- and post-therapy to assess the change in cancer stem cell amount arising from a given therapy or regimen.

In other embodiments using flow cytometry of a sample, the cells in the sample may be treated with a substrate for aldehyde dehydrogenase that becomes fluorescent when catalyzed by this enzyme. For instance, the sample can be treated with BODIPY®-aminoacetaldehyde which is commercially available from StemCell Technologies Inc. as Aldefluor®. Cancer stem cells express high levels of aldehyde dehydrogenase relative to bulk cancer cells and therefore become brightly fluorescent upon reaction with the substrate. The cancer stem cells, which become fluorescent in this type of experiment, can then be detected and counted using a standard flow cytometer. In this way, flow cytometry could be used to measure cancer stem cell amount pre- and post-therapy to assess the change in cancer stem cell amount arising from a given therapy or regimen.

In other embodiments, a sample (e.g., a tumor or normal tissue sample, blood sample or bone marrow sample) obtained from the patient is cultured in in vitro systems to assess the cancer stem cell population or amount of cancer stem cells. For example, tumor samples can be cultured on soft agar, and the amount of cancer stem cells can be correlated to the ability of the sample to generate colonies of cells that can be visually counted. Colony formation is considered a surrogate measure of stem cell content, and thus, can be used to quantitate the amount of cancer stem cells. For instance, with hematological cancers, colony-forming assays include colony forming cell (CFC) assays, long-term culture initiating cell (LTC-IC) assays, and suspension culture initiating cell (SC-IC) assays. In this way, the colony-forming or related assay could be used to measure cancer stem cell amount pre- and post-therapy to assess the change in cancer stem cell amount arising from a given therapy or regimen.

In other embodiments, sphere formation is measured to determine the amount of cancer stem cells in a sample (e.g., cancer stem cells form three-dimensional clusters of cells, called spheres) in appropriate media that is conducive to forming spheres. Spheres can be quantitated to provide a measure of cancer stem cells. See Singh et al., “Identification of a Cancer Stem Cell from Human Brain Tumors,” Cancer Res 63: 5821-5828 (2003). Secondary spheres can also be measured. Secondary spheres are generated when the spheres that form from the patient sample are broken apart, and then allowed to reform. In this way, the sphere-forming assay could be used to measure cancer stem cell amount pre- and post-therapy to assess the change in cancer stem cell amount arising from a given therapy or regimen.

In other embodiments, the amount of cancer stem cells in a sample can be determined with a cobblestone assay. Cancer stem cells from certain hematological cancers form “cobblestone areas” (CAs) when added to a culture containing a monolayer of bone marrow stromal cells. For instance, the amount of cancer stem cells from a leukemia sample can be assessed by this technique. The tumor samples are added to the monolayer of bone marrow stromal cells. The leukemia cancer stem cells, more so than the bulk leukemia cells, have the ability to migrate under the stromal layer and seed the formation of a colony of cells which can be seen visually under phase contrast microscopy in approximately 10-14 days as CAs. The number of CAs in the culture is a reflection of the leukemia cancer stem cell content of the tumor sample, and is considered a surrogate measure of the amount of stem cells capable of engrafting the bone marrow of immunodeficient mice. This assay can also be modified so that the CAs can be quantitated using biochemical labels of proliferating cells instead of manual counting, in order to increase the throughput of the assay. See Chung et al., “Enforced expression of an Flt3 internal tandem duplication in human CD34+ cells confers properties of self-renewal and enhanced erythropoiesis.” Blood 105(1):77-84 (2005). In this way, the cobblestone assay could be used to measure cancer stem cell amount pre- and post-therapy to assess the change in cancer stem cell amount arising from a given therapy or regimen.

In other embodiments, a sample (e.g., a tumor or normal tissue sample, blood sample or bone marrow sample) obtained from the patient is analyzed in in vivo systems to determine the cancer stem cell population or amount of cancer stem cells. In certain embodiments, for example, in vivo engraftment is used to quantitate the amount of cancer stem cells in a sample. In vivo engraftment involves implantation of a human specimen with the readout being the formation of tumors in an animal such as in immunocompromised or immunodeficient mice (such as NOD/SCID mice). Typically, the patient sample is cultured or manipulated in vitro and then injected into the mice. In these assays, mice can be injected with a decreasing amount of cells from patient samples, and the frequency of tumor formation can be plotted vs. the amount of cells injected to determine the amount of cancer stem cells in the sample. Alternatively, the rate of growth of the resulting tumor can be measured, with larger or more rapidly advancing tumors indicating a higher cancer stem cell amount in the patient sample. In this way, an in vivo engraftment model/assay could be used to measure cancer stem cell amount pre- and post-therapy to assess the change in cancer stem cell amount arising from a given therapy or regimen.

In certain in vivo techniques, an imaging agent, or diagnostic moiety, is used which binds to molecules on cancer cells or cancer stem cells, e.g., cancer cell or cancer stem cell surface antigens. For instance, a fluorescent tag, radionuclide, heavy metal, or photon-emitter is attached to an antibody (including an antibody fragment) that binds to a cancer stem cell surface antigen. Exemplary cancer stem cell surface antigens are listed above in Table 2. The medical practitioner can infuse the labeled antibody into the patient either prior to, during, or following treatment, and then the practitioner can place the patient into a total body scanner/developer which can detect the attached label (e.g., fluorescent tag, radionuclide, heavy metal, photon-emitter). The scanner/developer (e.g., CT, MRI, or other scanner, e.g. detector of fluorescent label, that can detect the label) records the presence, amount/quantity, and bodily location of the bound antibody. In this manner, the mapping and quantitation of tag (e.g. fluorescence, radioactivity, etc.) in patterns (i.e., different from patterns of normal stem cells within a tissue) within a tissue or tissues indicates the treatment efficacy within the patient's body when compared to a reference control such as the same patient at an earlier time point or a patient or healthy individual who has no detectable cancer. For example, a large signal (relative to a reference range or a prior treatment date, or prior to treatment) at a particular location indicates the presence of cancer stem cells. If this signal is increased relative to a prior date it suggests a worsening of the disease and failure of therapy or regimen. Alternatively, a signal decrease indicates that the therapy or regimen has been effective.

In a specific embodiment, the amount of cancer stem cells is detected in vivo in a subject according to a method comprising the steps of: (a) administering to the subject an effective amount of a labeled cancer stem cell marker binding agent that specifically binds to a cell surface marker found on the cancer stem cells, and (b) detecting the labeled agent in the subject following a time interval sufficient to allow the labeled agent to concentrate at sites in the subject where the cancer stem cell surface marker is expressed. In accordance with this embodiment, the cancer stem cell surface marker-binding agent is administered to the subject according to any suitable method in the art, for example, parenterally (such as intravenously), or intraperitoneally. In accordance with this embodiment, the effective amount of the agent is the amount which permits the detection of the agent in the subject. This amount will vary according to the particular subject, the label used, and the detection method employed. For example, it is understood in the art that the size of the subject and the imaging system used will determine the amount of labeled agent needed to detect the agent in a subject using an imaging means. In the case of a radiolabeled agent for a human subject, the amount of labeled agent administered is measured in terms of radioactivity, for example from about 5 to 20 millicuries of ⁹⁹Tc. The time interval following the administration of the labeled agent which is sufficient to allow the labeled agent to concentrate at sites in the subject where the cancer stem cell surface marker is expressed will vary depending on several factors, for example, the type of label used, the mode of administration, and the part of the subject's body that is imaged. In a particular embodiment, the time interval that is sufficient is 6 to 48 hours, 6 to 24 hours, or 6 to 12 hours. In another embodiment the time interval is 5 to 20 days or 5 to 10 days. The presence of the labeled cancer stem cell surface marker-binding agent can be detected in the subject using imaging means known in the art. In general, the imaging means employed depend upon the type of label used. Skilled artisans will be able to determine the appropriate means for detecting a particular label. Methods and devices that may be used include, but are not limited to, computed tomography (CT), whole body scan such as position emission tomography (PET), magnetic resonance imaging (MRI), and sonography. In a specific embodiment, the cancer stem cell surface marker-binding agent is labeled with a radioisotope and is detected in the patient using a radiation responsive surgical instrument (Thurston et al., U.S. Pat. No. 5,441,050). In another embodiment, the cancer stem cell surface marker-binding agent is labeled with a fluorescent compound and is detected in the patient using a fluorescence responsive scanning instrument. In another embodiment, the cancer stem cell surface marker-binding agent is labeled with a positron emitting metal and is detected in the patient using positron emission-tomography. In yet another embodiment, the cancer stem cell surface marker-binding agent is labeled with a paramagnetic label and is detected in a patient using magnetic resonance imaging (MRI).

Any in vitro or in vivo (ex vivo) assays known to those skilled in the art that can detect and/or quantify cancer stem cells can be used to monitor cancer stem cells in order to evaluate the prophylactic and/or therapeutic utility of a cancer therapy or regimen disclosed herein for cancer or one or more symptoms thereof; or these assays can be used to assess the prognosis of a patient. The results of these assays then may be used to possibly maintain or alter the cancer therapy or regimen.

The amount of cancer stem cells in a specimen can be compared to a predetermined reference range and/or an earlier amount of cancer stem cells previously determined for the subject (either prior to, or during therapy) in order to gauge the subject's response to the treatment regimens described herein. In a specific embodiment, a stabilization or reduction in the amount of cancer stem cells relative to a predetermined reference range and/or earlier cancer stem cell amount previously determined for the subject (prior to, during and/or after therapy) indicates that the therapy or regimen was effective and thus possibly an improvement in the subject's prognosis, whereas an increase relative to the predetermined reference range and/or cancer stem cell amount detected at an earlier time point indicates that the therapy or regimen was ineffective and thus possibly the same or a worsening in the subject's prognosis. The cancer stem cell amount can be used with other standard measures of cancer to assess the prognosis of the subject and/or efficacy of the therapy or regimen: such as response rate, durability of response, relapse-free survival, disease-free survival, progression-free survival, and overall survival. In certain embodiments, the dosage, frequency and/or duration of administration of a therapy is modified as a result of the determination of the amount or change in relative amount of cancer stem cells at various time points which may include prior to, during, and/or following therapy.

The present invention also relates to methods for determining that a cancer therapy or regimen is effective at targeting and/or impairing cancer stem cells by virtue of monitoring cancer stem cells over time and detecting a stabilization or decrease in the amount of cancer stem cells during and/or following the course of the cancer therapy or regimen.

In a certain embodiment, a therapy or regimen may be marketed as an anti-cancer stem cell therapy or regimen based on the determination that a therapy or regimen is effective at targeting and/or impairing cancer stem cells by virtue of having monitored or detected a stabilization or decrease in the amount of cancer stem cells during therapy.

5.5 Methods of Monitoring Cancer Cells

As part of the prophylactically effective regimens and/or therapeutically effective regimens of the invention, the amount of cancer cells (alone or in combination with the amount of cancer stem cells) can be monitored/assessed using standard techniques known to one of skill in the art. In certain embodiments of the prophylactically effective regimens and/or therapeutically effective regimens of the invention, the regimens result in a stabilization or reduction in the amount (expressed, e.g., as a percentage) of cancer cells in the subject. In one embodiment, the subject undergoing the regimen is monitored to determine whether the regimen has resulted in a stabilization or reduction in the amount (expressed, e.g., as a percentage) of cancer cells in the subject.

In some embodiments, the amount of cancer cells is assessed in a subject using techniques described herein or known to one of skill in the art. In other embodiments, the amount of cancer cells is detected in a sample. Such samples include, but are not limited to, biological samples and samples derived from a biological sample. In certain embodiments, in addition to the biological sample itself or in addition to material derived from the biological sample such as cells, the sample used in the methods of this invention comprises added water, salts, glycerin, glucose, an antimicrobial agent, paraffin, a chemical stabilizing agent, heparin, an anticoagulant, or a buffering agent. In certain embodiments, the biological sample is blood, serum, urine, bone marrow or interstitial fluid. In another embodiment, the sample is a tissue sample. In a particular embodiment, the tissue sample is breast, colon, lung, liver, ovarian, pancreatic, prostate, renal, bone or skin tissue. In a specific embodiment, the tissue sample is a biopsy, including a tumor biopsy. The amount of biological sample taken from the subject will vary according to the type of biological sample and the method of detection to be employed. In a particular embodiment, the biological sample is blood, serum, or urine and the amount of blood, serum, or urine taken from the subject is 0.1 ml, 0.5 ml, 1 ml, 5 ml, 10 ml or more. In another embodiment, the biological sample is a tissue and the amount of tissue taken from the subject is less than 10 milligrams, less than 25 milligrams, less than 50 milligrams, less than 1 gram, less than 5 grams, less than 10 grams, less than 50 grams, or less than 100 grams.

In accordance with the methods of the invention, a sample derived from a biological sample is one in which the biological sample has been subjected to one or more pretreatment steps prior to the detection and/or measurement of the cancer cell population in the sample. In certain embodiments, a biological fluid is pretreated by centrifugation, filtration, precipitation, dialysis, or chromatography, or by a combination of such pretreatment steps. In other embodiments, a tissue sample is pretreated by freezing, chemical fixation, paraffin embedding, dehydration, permeabilization, or homogenization followed by centrifugation, filtration, precipitation, dialysis, or chromatography, or by a combination of such pretreatment steps. In certain embodiments, the sample is pretreated by removing cells other than cancer cells from the sample, or removing debris from the sample prior to the determination of the amount of cancer cells in the sample according to the methods of the invention.

The samples for use in the methods of this invention may be taken from any animal subject, preferably a mammal, most preferably a human. The subject from which a sample is obtained and utilized in accordance with the methods of this invention includes, without limitation, an asymptomatic subject, a subject manifesting or exhibiting 1, 2, 3, 4 or more symptoms of cancer, a subject clinically diagnosed as having cancer, a subject predisposed to cancer, a subject suspected of having cancer, a subject undergoing therapy for cancer, a subject that has been medically determined to be free of cancer (e.g., following therapy for the cancer), a subject that is managing cancer, or a subject that has not been diagnosed with cancer.

In certain embodiments, the amount of cancer cells is assessed in a subject or a sample from a subject at least 1, 2, 4, 6, 8, 10, 12, 14, 15, 16, 18, 20, or 30, 60, 90 days 6 months, 9 months, 12 months, >12 months after the subject begins receiving the regimen. In certain embodiments, the amount of cancer cells is assessed after a number of doses (e.g., after 1, 2, 5, 10, 20, 30 or more doses of a therapy). In other embodiments, the amount of cancer cells is assessed after 2 weeks, 1 month, 2 months, 1 year, 2 years, 3 years, 4 years or more after receiving one or more therapies.

The amount of cancer cells in a sample can be expressed as the percentage of, e.g., overall cells in the sample. In some embodiments, the sample is a blood sample or bone marrow sample, wherein the amount of cancer cells per unit of volume (e.g., 1 mL) or other measured unit (e.g., per unit field in the case of a histological analysis) is quantitated. The cancer cell population, in certain embodiments, can be determined as a percentage of the total blood cells.

In other embodiments, the sample from the patient is a tissue sample (e.g., a biopsy from a subject with or suspected or having cancerous tissue), where the amount of cancer cells can be measured, for example, by immunohistochemistry or on the basis of the amount of cancer cells per unit weight of the tissue.

The amount of cancer cells in the test sample can be compared with the amount of cancer cells measured in a reference sample(s) to assess the efficacy of the regimen. In one embodiment, the reference sample is a sample from the subject undergoing therapy, at an earlier time point (e.g., prior to receiving the regimen as a baseline reference sample, or at an earlier time point while receiving the therapy). In this embodiment, the therapy desirably results in a decrease in the amount of cancer cells in the test sample as compared with the reference sample. In another embodiment, the reference sample is obtained from a healthy, subject who has no detectable cancer, or from a patient that is in remission for the same type of cancer. In this embodiment, the therapy desirably results in the test sample having an equal amount of cancer cells as detected in the reference sample (e.g., no detectable cancer cells).

If the reduction in the amount of cancer cells is judged too small, then the medical practitioner has a number of options to adjust the regimen. For instance, the medical practitioner can then either increase the dosage of the therapy administered, the frequency of the administration, the duration of administration, combine the therapy with another therapy(ies), halt the therapy, or any combination thereof.

The amount of cancer cells can be monitored/assessed using standard techniques known to one of skill in the art. Cancer cells can be monitored by, e.g., obtaining a sample, such as a tumor sample, blood sample or bone marrow sample, from a subject and detecting cancer cells in the sample. The amount of cancer cells in a sample (which may be expressed as a percentage) can be assessed by detecting the expression of antigens on cancer cells and/or by detecting the proliferation of cancer cells. Techniques known to those of skilled in the art can be used for measuring these activities. For example, cellular proliferation can be assayed by 3H-thymidine incorporation assays and trypan blue cell counts. Antigen expression can be assayed, for example, by immunoassays including, but are not limited to western blots, immunohistochemistry radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, fluorescence-activated cell sorter (FACS) analysis, flow cytometry and immunofluorescence.

The amount of cancer cells can be compared to a predetermined reference range and/or an earlier amount of cancer cells determined for the subject to gauge the subject's response to the regimens described herein. In a specific embodiment, a reduction in the amount of cancer cells relative to a predetermined reference range and/or earlier cancer cell amount determined for the subject indicate an improvement in the subject's prognosis or response to a therapy, whereas an increase relative to the predetermined reference range and/or earlier cancer cell numbers indicates the same or worse prognosis, or failure to respond to a therapy. In certain embodiments, the dosage, frequency and/or duration of administration of a therapy is modified as a result of the change in the amount of cancer cells.

In some embodiments, the cancer cell population can be monitored/assessed using gross measurements of the cancer cell population. For example, in some embodiments, the cancer cell population is determined using imaging methods such as computed tomography (CT), magnetic resonance imaging (MRI), ultrasound, X-ray imaging, mammography, radionuclide imaging, PET scan, palpitation, direct measurement (e.g. with a ruler) or bone scans.

In embodiments of the invention comprising treatment of solid tumors, the bulk size of the tumor may provide an estimate of the cancer cell population. A number of known methods can be used to assess the bulk size of the tumor. Non-limiting examples of such methods include imaging methods (e.g., computed tomography (CT), magnetic resonance imaging (MRI), PET scans, palpitation, direct measurement (e.g. with a ruler), ultrasound, X-ray imaging, mammography, bone scans and radioisotope imaging), visual methods (e.g., colonoscopy, bronchoscopy, endoscopy), physical examination (e.g., prostate examination, breast examination, lymph nodes examination, abdominal examination, general palpation), blood tests (e.g., prostate specific antigen (PSA) test, carcinoembryonic antigen (CEA) test, cancer antigen (CA)-125 test, alpha-fetoprotein (AFP)), bone marrow analyses (e.g., in cases of hematological malignancies), histopathology, cytology and flow cytometry.

In some embodiments, the bulk tumor size can be measured by assessments based on the size of tumor lesions determined from imaging methods. In specific embodiments, the assessments are performed in accordance with the Response Evaluation Criteria In Solid Tumors (RECIST) Guidelines, which are set forth in Therasse, P. et al., “New Guidelines to Evaluate the Response to Treatment in Solid Tumors,” J. of the Nat. Canc. Inst. 92(3), 205-216 (2000). For instance, in specific embodiments, lesions in the subject that are representative of bulk tumor size are selected so that they are at least=20 mm in their longest diameter at baseline (prior to treatment) when conventional imaging techniques are used (e.g., conventional CT scan, MRI or x-ray) and lesions that are at least =10 mm in their longest diameter at baseline should be selected when spiral CT scanning is used.

5.6 Methods of Monitoring Lymphocyte Cell Count, Neutrophil Cell Count, Platelet Count and Hemoglobin

As part of the prophylactically effective regimens and/or therapeutically effective regimens of the invention, the peripheral blood lymphocyte counts can be monitored/assessed using standard techniques known to one of skill in the art. Peripheral blood lymphocytes counts in a subject can be determined by, e.g., obtaining a sample of peripheral blood from said subject, separating the lymphocytes from other components of peripheral blood such as plasma using e.g., Ficoll-Hypaque (Pharmacia) gradient centrifugation, and counting the lymphocytes using trypan blue. Peripheral blood T-cell counts in subject can be determined by, e.g., separating the lymphocytes from other components of peripheral blood such as plasma using, e.g., a use of Ficoll-Hypaque (Pharmacia) gradient centrifugation. Labeling the T-cells with an antibody directed to a T-cell antigen such as CD3, CD4, and CD8 which is conjugated to a FACS detectable agent, such as FITC or phycoerythrin, and measuring the number of T-cells by FACS. Further, the effect on a particular subset of T cells (e.g., CD2⁺, CD4⁺, CD8⁺, CD25⁺, CD45RO⁺, CD45RA⁺, or CD8⁺RA⁺) or NK cells can be determined using standard techniques known to one of skill in the art such as FACS.

The subject's absolute neutrophil count (ANC) can be monitored/assessed using standard techniques known to one of skill in the art. In some embodiments, the regimen includes monitoring the patient's ANC in order to avoid the risk of the patient developing neutropenia.

The ANC can be calculated from measurements of the total number of white blood cells (WBC) and the numbers of neutrophils and bands (immature neutrophils). The ANC can be determined manually by trained medical technologists or by automated ANC results obtained from automated hematology analyzers.

The subject's platelet count (PLT) can be monitored/assessed using standard techniques known to one of skill in the art. In some embodiments, the regimen includes monitoring the patient's platelet count in order to avoid the risk of the patient developing thrombocytopenia or becoming blood transfusion dependent. Transfusions can be given as determined by the physician.

The subject's hemoglobin (Hgb) can be monitored/assessed using standard techniques known to one of skill in the art. In some embodiments, the regimen includes monitoring the patient's hemoglobin in order to avoid the risk of the patient developing anemia or becoming transfusion dependent. Transfusions or growth factors (e.g. erythropoietin) can be given as determined by the physician.

5.7 Biological Assays

5.7.1 In Vitro Assays

The compounds, pharmaceutical compositions and regimens of the invention can be tested in vitro and/or in vivo for their ability to reduce the number of cancer cells and/or cancer stem cells, or inhibit their proliferation. The ability of a compound or a regimen of the invention to reduce the number of cancer cells, cancer stem cells and/or immune cells (e.g., lymphocytes) or inhibit their proliferation can be assessed by: detecting the expression of antigens on cancer cells, cancer stem cells, and immune cells; detecting the proliferation or viability of cancer cells, cancer stem cells and immune cells; detecting the effector function of cancer cells and cancer stem cells. Techniques known to those of skilled in the art can be used for measuring these activities. For example, cellular proliferation can be assayed by 3H-thymidine incorporation assays and trypan blue cell counts. Antigen expression can be assayed, for example, by immunoassays including, but are not limited to, competitive and non-competitive assay systems using techniques such as western blots, immunohistochemistry radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, flow cytometry, and FACS analysis.

A compound, pharmaceutical composition, or regimen of the invention is preferably tested in vitro and then in vivo for the desired therapeutic or prophylactic activity prior to use in humans. For example, assays which can be used to determine whether administration of a specific compound is indicated include cell culture assays in which a patient tissue sample (e.g., a cancer stem cell or cancer cell) is grown in culture and exposed to, or otherwise contacted with, a compound of the invention, and the effect of such compound upon the tissue sample is observed. The tissue sample can be obtained by biopsy from the patient. This test allows the identification of the therapeutically most effective therapy (e.g., prophylactic or therapeutic agent) for each individual patient.

Determination of cell viability using the XTT assay: In some cases, CD34+ cells are isolated from human cord blood using magnetic beads coated with anti-CD34 antibody. Isolated cells are then counted and aliquoted into 96-well plates and then incubated in the presence of varying concentrations of cantharidin or norcantharidin. Cell viability it measured by the addition of the XTT calorimetric reagent. Viability is determined by the absorbance of treated cultures at approximately 450-500 nm compared to untreated cultures. In other cases, the cells used in the assay may be a leukemia cell line, such as MV4; 11. The assay can also be used to determine the time course of cell killing by various compounds by performing the XTT assay on cultures that are incubated with the compounds for varying periods of time.

Cobblestone assay: The cobblestone area-forming cell (CAFC) assay exploits a reproducible visual end point for the quantitation of cancer stem cells. Leukemia samples are added to adherent cultures of stromal cells, some embodiments MS-5 stromal cells. The cancer stem cells in the culture will migrate below the MS-5 stromal cells and form a colony of cells called a cobblestone that can be visual quantitated. To test the effect of cantharidin or norcantharidin on the cancer stem cell population using this assay, cells are first cultured in the presence of the drug. In some embodiments the cells are cultured for 16 hours. After this incubation, the cells are added to the stromal cultures. A reduction in the cobblestone area formation in cultures that were treated with the drug compared to the untreated cells represents cancer stem cell activity for the drug.

5.7.2 In Vivo Assays

The compounds, pharmaceutical compositions, and regimens of the invention can be tested in suitable animal model systems prior to use in humans. Such animal model systems include, but are not limited to, rats, mice, chicken, cows, monkeys, pigs, dogs, rabbits, etc. Any animal system well-known in the art may be used. Several aspects of the procedure may vary; said aspects include, but are not limited to, the temporal regime of administering the therapeutic modalities (e.g., prophylactic and/or therapeutic agents), whether such therapeutic modalities are administered separately or as an admixture, and the frequency of administration of the therapeutic modalities.

Animal models for cancer can be used to assess the efficacy of a compound or a combination therapy of the invention. Examples of animal models for lung cancer include, but are not limited to, lung cancer animal models described by Zhang & Roth (1994, In Vivo 8(5):755-69) and a transgenic mouse model with disrupted p53 function (see, e.g., Morris et al. J La. State Med. Soc. 1998, 150(4):179-85). An example of an animal model for breast cancer includes, but is not limited to, a transgenic mouse that overexpresses cyclin D1 (see, e.g., Hosokawa et al., Transgenic Res. 2001, 10(5), 471-8. An example of an animal model for colon cancer includes, but is not limited to, a TCR b and p53 double knockout mouse (see, e.g., Kado et al., Cancer Res. 2001, 61(6):2395-8). Examples of animal models for pancreatic cancer include, but are not limited to, a metastatic model of PancO2 murine pancreatic adenocarcinoma (see, e.g., Wang et al., Int. J. Pancreatol. 2001, 29(1):37-46) and nu-nu mice generated in subcutaneous pancreatic tumours (see, e.g., Ghaneh et al., Gene Ther. 2001, 8(3):199-208). Examples of animal models for non-Hodgkin's lymphoma include, but are not limited to, a severe combined immunodeficiency (“SCID”) mouse (see, e.g., Bryant et al., Lab Invest. 2000, 80(4), 553-73) and an IgHmu-HOX 11 transgenic mouse (see, e.g., Hough et al., Proc. Natl. Acad. Sci. USA 1998, 95(23), 13853-8. An example of an animal model for esophageal cancer includes, but is not limited to, a mouse transgenic for the human papillomavirus type 16 E7 oncogene (see, e.g., Herber et al., J. Virol. 1996, 70(3):1873-81). Examples of animal models for colorectal carcinomas include, but are not limited to, APC mouse models (see, e.g., Fodde & Smits, Trends Mol. Med. 2001, 7(8):369-73 and Kuraguchi et al., Oncogene 2000, 19(50), 5755-63).

In some embodiments of the invention, the efficacy of the therapeutic regimen in reducing the amount of cancer stem cells in animals (including humans) undergoing treatment can be evaluated using in vivo techniques. In these embodiments, an imaging agent is used which binds to biological molecules on cancer cells or cancer stem cells, e.g., cancer stem cell surface antigens. For instance, a fluorescent tag, heavy metal, photon emitter or radionuclide is covalently attached to an antibody (including an antibody fragment) that specifically binds to a cancer stem cell surface antigen. Exemplary cancer stem cell surface antigens are listed herein. The medical practitioner can infuse the labeled antibody into the patient either prior to, during, or following untreated or undergoing treatment, and then the practitioner can place the patient into a total body scanner/developer which can detect the attached label (e.g., fluorescent tag or radionuclide). The scanner/developer (e.g., CT or MRI, other scanner, e.g., detector of fluorescent label, that can detect the label) records the presence, amount/quantity and bodily location, and amount of the bound antibody based on the signal generated by the imaging agent. In this manner, the mapping and quantitation of tag (e.g., fluorescence, radioactivity, ect.) in patterns (i.e., different from patterns of normal stem cells within a tissue) within a tissue or tissues indicates the treatment efficacy within the patient's body when compared to a reference control such as the same patient at an earlier time point or a patient who has no detectable cancer. For example, a large signal (relative to a reference range or a prior treatment date, or to prior treatment) at a particular location indicates the presence of cancer stem cells. If this signal is increased relative to a prior treatment it suggests a worsening of the disease and failure of therapy or regimen. Alternatively, a signal decrease indicates that therapy or regimen is working.

Similarly, in some embodiments of the invention, the efficacy of the therapeutic regimen in reducing the amount of cancer cells in animals (including humans) undergoing treatment can be evaluated using in vivo techniques. In one embodiment, the medical practitioner performs the imaging technique with labeled molecule that specifically binds the surface of a cancer cell, e.g., a cancer cell surface antigen. See Section 5.4, supra, lists certain cancer cell surface antigens. In this manner, the mapping and quantitation of tag (e.g., fluorescence, radioactivity) in patterns within a tissue or tissues indicates the treatment efficacy within the body of the patient undergoing treatment.

In a specific embodiment, the amount of cancer stem cells is detected in vivo in a subject according to a method comprising the steps of: (a) administering to the subject an effective amount of a labeled cancer stem cell marker binding agent that specifically binds to a cell surface marker found on the cancer stem cells, and (b) detecting the labeled agent in the subject following a time interval sufficient to allow the labeled agent to concentrate at sites in the subject where the cancer stem cell surface marker is expressed. In accordance with this embodiment, the cancer stem cell surface marker-binding agent is administered to the subject according to any suitable method in the art, for example, parenterally (e.g. intravenously), or intraperitoneally. In accordance with this embodiment, the effective amount of the agent is the amount which permits the detection of the agent in the subject. This amount will vary according to the particular subject, the label used, and the detection method employed. For example, it is understood in the art that the size of the subject and the imaging system used will determine the amount of labeled agent needed to detect the agent in a subject using imaging. In the case of a radiolabeled agent for a human subject, the amount of labeled agent administered is measured in terms of radioactivity, for example from about 5 to 20 millicuries of 99Tc. The time interval following the administration of the labeled agent which is sufficient to allow the labeled agent to concentrate at sites in the subject where the cancer stem cell surface marker is expressed will vary depending on several factors, for example, the type of label used, the mode of administration, and the part of the subject's body that is imaged. In a particular embodiment, the time interval that is sufficient is 6 to 48 hours, 6 to 24 hours, or 6 to 12 hours. In another embodiment the time interval is 5 to 20 days or 5 to 10 days. The presence of the labeled cancer stem cell surface marker-binding agent can be detected in the subject using imaging means known in the art. In general, the imaging means employed depend upon the type of label used. Skilled artisans will be able to determine the appropriate means for detecting a particular label. Methods and devices that may be used include, but are not limited to, computed tomography (CT), whole body scan such as position emission tomography (PET), magnetic resonance imaging (MRI), fluorescence, chemiluminescence, and sonography. In a specific embodiment, the cancer stem cell surface marker-binding agent is labeled with a radioisotope and is detected in the patient using a radiation responsive surgical instrument (Thurston et al., U.S. Pat. No. 5,441,050). In another embodiment, the cancer stem cell surface marker-binding agent is labeled with a fluorescent compound and is detected in the patient using a fluorescence responsive scanning instrument. In another embodiment, the cancer stem cell surface marker-binding agent is labeled with a positron emitting metal and is detected in the patient using positron emission-tomography. In yet another embodiment, the cancer stem cell surface marker-binding agent is labeled with a paramagnetic label and is detected in a patient using magnetic resonance imaging (MRI).

Further, any assays known to those skilled in the art can be used to evaluate the prophylactic and/or therapeutic utility of a compound or pharmaceutical composition disclosed herein for cancer or one or more symptoms thereof.

5.7.3 Assessing Toxicity

The toxicity and/or efficacy of compounds, pharmaceutical compositions, and regimens of the invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Therapeutic regimens that exhibit large therapeutic indices are preferred. While therapeutic regimens that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the therapies for use in humans. The dosage of such agents lies preferably within a range of circulating concentrations that include the ED₅₀ with little or no toxicity to normal tissues. The dosage may vary within this range depending upon the dosage form employed and the route of administration utilized. For any therapy used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels of compounds in plasma may be measured, for example, by high performance liquid chromatography.

5.8 Articles of Manufacture

The present invention also encompasses a finished packaged and labeled pharmaceutical product. This article of manufacture includes the appropriate unit dosage form in an appropriate vessel or container such as a glass vial or other container that is hermetically sealed. The pharmaceutical product may contain, for example, a compound of the invention in a unit dosage form in a first container, and in a second container, sterile water for injection. In some embodiments the ways of injection include, but are not limited, to intravenous, subcutaneous, intradermal, intramuscular and intratumoral. Alternatively, the unit dosage form may be a solid suitable for oral, transdermal, intranasal, rectal or topical delivery.

In a specific embodiment, the unit dosage form is suitable for intravenous, intramuscular, intranasal, oral, topical, rectal or subcutaneous delivery. Thus, the invention encompasses solutions, preferably sterile, suitable for each delivery route.

As with any pharmaceutical product, the packaging material and container are designed to protect the stability of the product during storage and shipment. Further, the products of the invention include instructions for use or other informational material that advise the physician, technician or patient on how to appropriately prevent or treat the disease or disorder in question. In other words, the article of manufacture includes instruction means indicating or suggesting a dosing regimen including, but not limited to, actual doses, monitoring procedures, cancer cell counts, cancer stem cell counts, and other monitoring information.

Specifically, the invention provides an article of manufacture comprising packaging material, such as a box, bottle, tube, vial, container, sprayer, insufflator, intravenous (i.v.) bag, envelope and the like; and at least one unit dosage form of a pharmaceutical agent contained within said packaging material, wherein said pharmaceutical agent comprises a compound of the invention, and wherein said packaging material includes instruction means which indicate that said compound can be used to prevent, manage, treat, and/or ameliorate one or more symptoms associated with cancer, or one or more symptoms thereof by administering specific doses and using specific dosing regimens as described herein.

In specific embodiments, the article of manufacture include labeled antibodies that selectively or specifically bind to stem cells, and preferably, that selectively or specifically bind to cancer stem cells. As such, the article contains a method to adjust the dosages used in the therapeutic regimens, and to monitor the efficacy of the therapeutic regimen.

### 6. EXAMPLES

### 6.1 Example 1

### Cytotoxicity of Cantharidin and Norcantharidin Against Leukemia Stem Cells

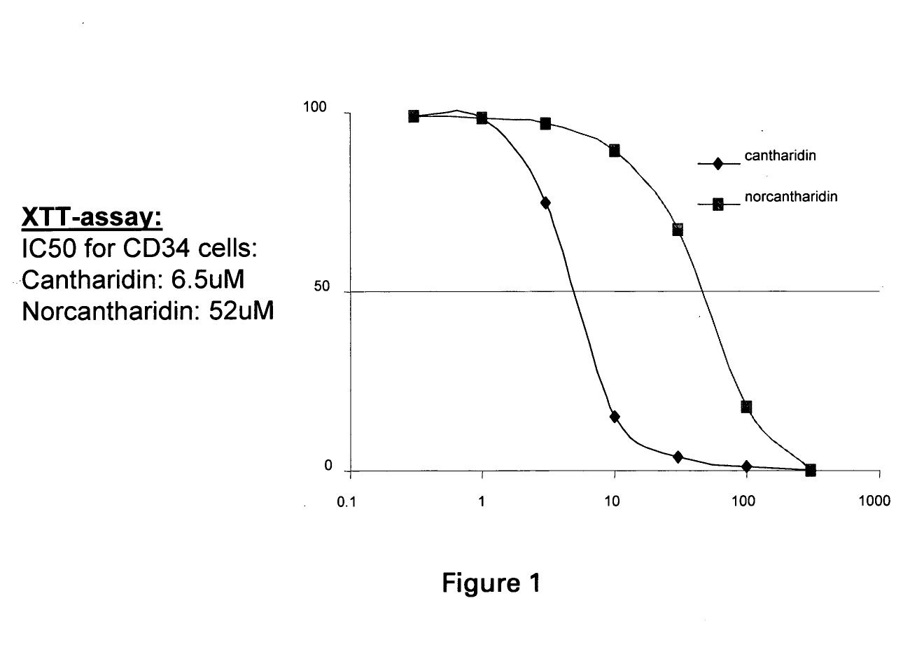

CD34⁺ cells were obtained from normal human cord blood by magnetic bead selection using anti-CD34 antibody coated beads. The cytotoxicity of cantharidin and norcantharidin against these cells was measured by a colorimetric assay using XTT (sodium 3′-{1-[(phenylamino)-carbonyl]-3,4-tetrazolium}-bis(4-methoxy-6-nitro) benzene-sulfonic acid hydrate). CD34⁺ cells were plated in 96-well plates and the following day, cells were exposed to varying doses of cantharidin and norcantharidin. 50 μL of 1 mg/mL XTT and 0.025 mM phenazine methosulfate (PMS) were added. The absorbance of the supernatant was measured at 450 and 630 nm of wells without drug (with cells) as 100% and wells without cells as 0%. FIG. 1 represents the dose response curve for CD34⁺ cells in the presence of cantharidin and norcantharidin. CD34⁺ cells were extremely sensitive to both compounds with an IC₅₀ of 6.5 μM for cantharidin, and, 52 μM for norcantharidin. In all experiments with cantharidin and norcantharidin, the range of concentrations tested were selected because they were estimated to encompass the estimated concentration of drug in human serum that is achieved when the drug is administered to patients.

### 6.2 Example 2

### Cobblestone Area Forming Cell Assay (CAFC) Comparing Cytotoxicity of Cantharidin and Norcantharidin Against Stem Cells from a Leukemic Patient and Stem Cells from Cord Blood (Normal Stem Cells)

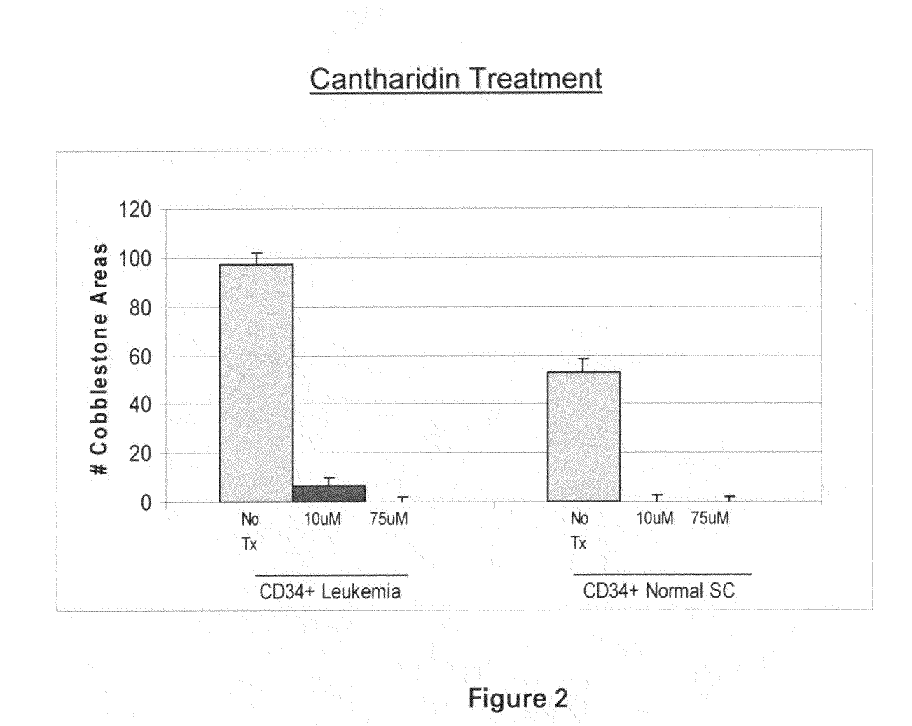

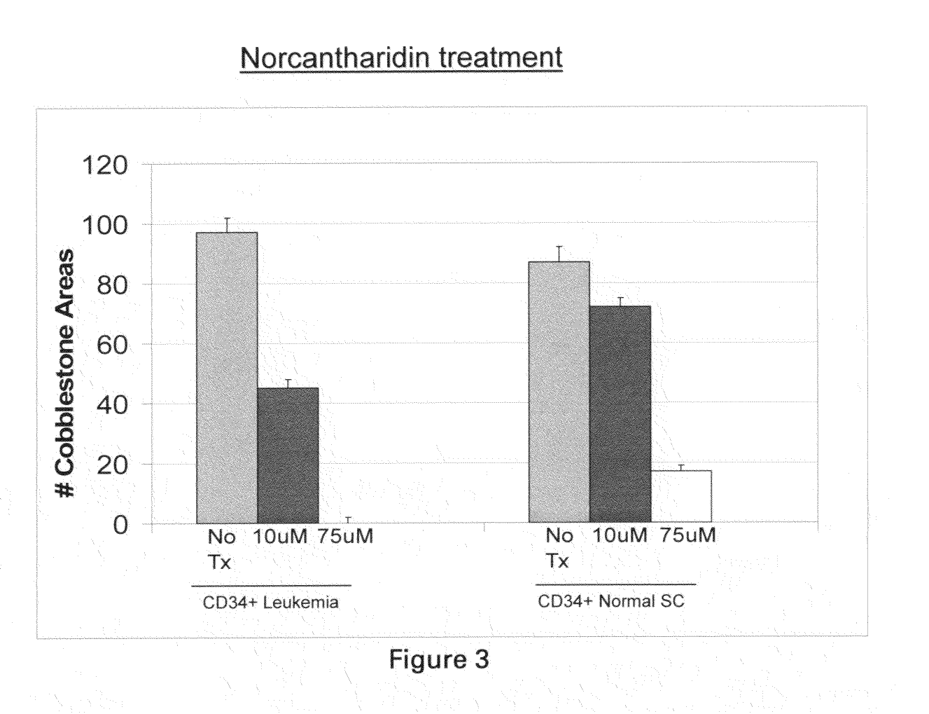

Cells were treated with 10 μM and 75 μM cantharidin or norcantharidin overnight. To assay for stem cells by the cobblestone area forming cell (CAFC) assay, CD34⁺ cells subsequent to drug treatment were co-cultured with the MS-5 monolayer in X-Eagle minimum essential medium (α-MEM) containing 10% heat-inactivated FCS, 10% horse serum, 1×10⁻⁶ M hydrocortisone, 2 mM L-glutamine, and 100 U/mL penicillin/streptomycin. After 5 weeks in culture, total cobblestone areas were counted. FIGS. 2 and 3 show bar graphs representing cobblestone area counts in the presence of no drug (control) and 10 and 75 μM of cantharidin and norcantharidin, respectively, using CD34+ cells obtained from a leukemia patient, and CD34+normal stem cells isolated from human cord blood. The control sample shows a significantly greater number of cobblestone areas as compared to samples where cells were treated with the drugs, thereby demonstrating potent activity of cantharidin and norcantharidin against leukemic cancer stem cells, and increased sensitivity of leukemia cancer stem cells relative to normal stem cells.

### 6.3 Example 3

### Cytotoxicity of Cantharidin and Norcantharidin Against Cancer Cells (MV4; 11 Leukemic Cells)

The cytotoxicity of Cantharidin and Norcantharidin against MV4; 11 leukemia cells was measured by a calorimetric assay using XTT. MV4; 11 cells were plated in 96-well plates and the following day, cells were exposed to varying doses of cantharidin and norcantharidin. After 5 days of exposure to drugs, 50 μL of 1 mg/mL XTT and 0.025 mM phenazine methosulfate (PMS) were added. The absorbance of the supernatant was measured at 450 and 630 nm of wells not treated with drug (with cells) as 100% and wells without cells as 0%. MV4; 11 cells were extremely sensitive to both compounds with IC₅₀ of 7.5 μM for cantharidin and 24 μM for norcantharidin.

### 6.4 Example 4

### Time Course Cell Viability Study of Cancer Cells (MV4;11 Leukemic Cells) in the Presence of Cantharidin and Norcantharidin

The XTT assay was used to determine the time course of killing of MV4; 11 cells incubated with cantharidin and norcantharidin over time.

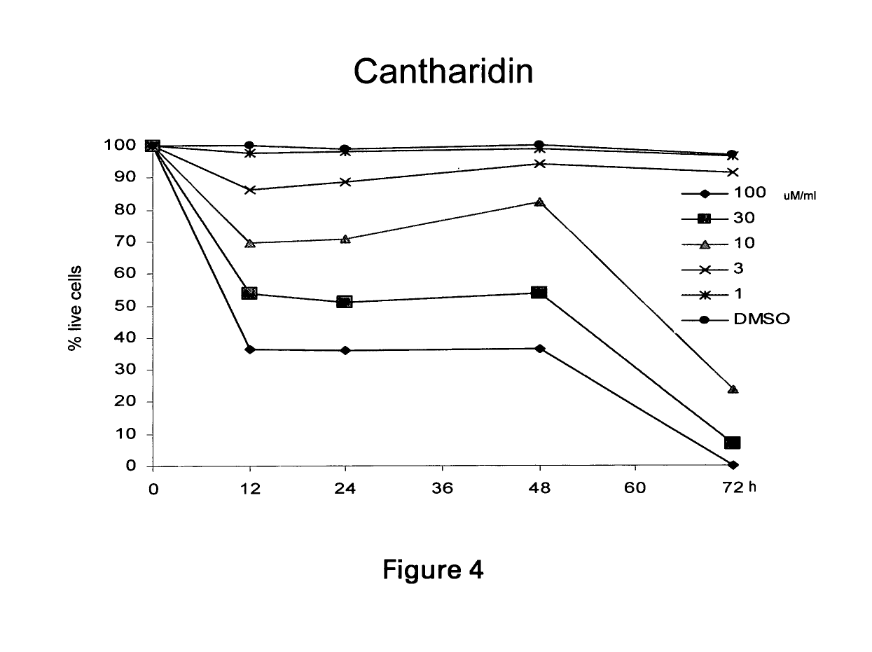

Cantharidin was dissolved in DMSO at different concentrations to ensure that final concentrations of DMSO are equivalent in all wells. To control for potential cytotoxic effects of DMSO itself, an equivalent amount of DMSO containing no drug was used as a control. FIG. 4 shows the time course of MV4; 11 cell viability in the presence of varying concentrations of cantharidin. This experiment demonstrates that over an extended period of time, similar cytotoxic effects can be achieved with lower concentrations of the drug, i.e. at 72 hours, in the presence of both 100 μM and 30 μM, cell viability is less than 10%.

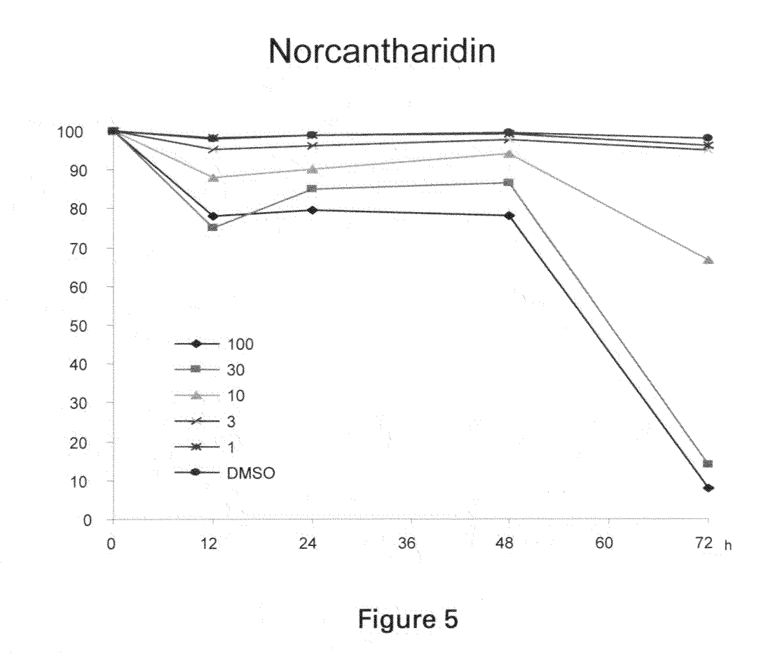

Norcantharidin was dissolved in DMSO at different concentrations to ensure that final concentrations of DMSO are equivalent in all wells. To control for potential cytotoxic effects of DMSO itself, an equivalent amount of DMSO containing no drug was used as a control. FIG. 5 shows the time course of MV4; 11 cell viability in the presence of varying concentrations of norcantharidin. This experiment demonstrates that over an extended period of time, similar cytotoxic effects can be achieved with lower concentrations of the drug, i.e. at 72 hours, in the presence of both, 100 μM and 30 μM, cell viability is at less than 10%.

### 6.5 Example 5

### Cobblestone Area Forming Cell Assay (CAFC) of Normal and Leukemia Stem Cells Treated with Cantharidin and Standard Chemotherapy

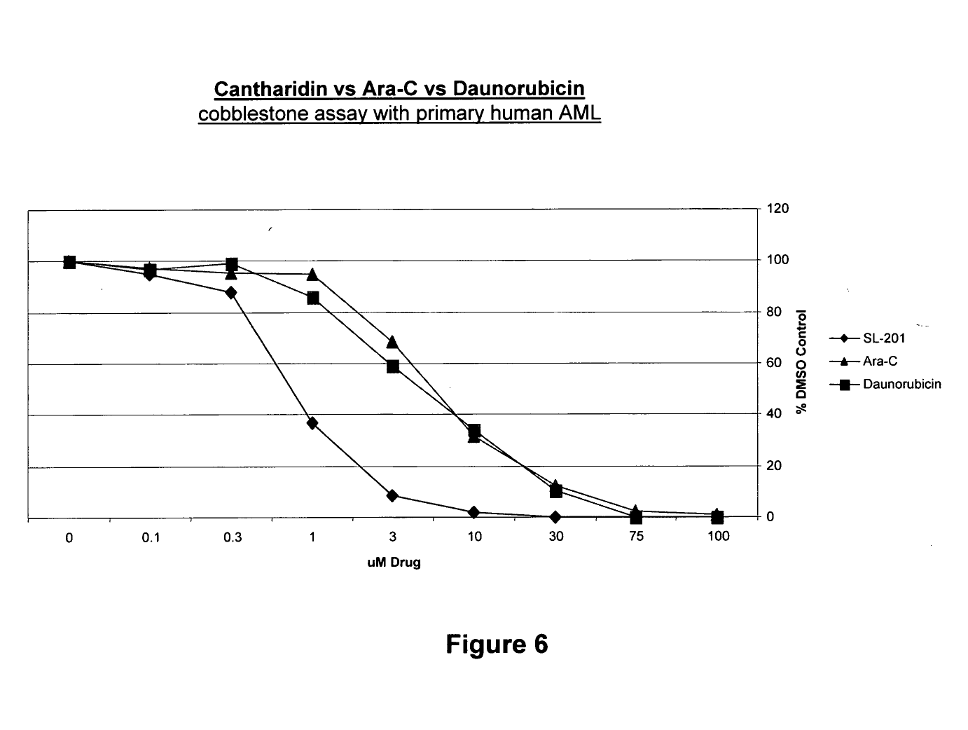

Samples of primary human cord blood as well as from the bone marrow of a patient with acute myelgenous leukemia (AML) were tested. Cantharidin and norcantharidin were dissolved in DMSO, and Ara-c and daunorubicin were prepared according to the supplier's instructions. In addition, untreated controls (diluent alone) for both normal and leukemic CD34+ cells were also tested. Cells were treated with varying concentrations of drug for 16 hours. To assay for stem cell activity by the cobblestone area forming cell (CAFC) assay, cells were washed in media, and then co-cultured with an MS-5 stromal monolayer in λ-Eagle minimum essential medium (α-MEM) containing 10% heat-inactivated FCS, 10% horse serum, 1×10⁻⁶ M hydrocortisone, 2 mM L-glutamine, and 100 U/mL penicillin/streptomycin. After 5 weeks in culture, total cobblestone areas were counted visually using a microscope. Leukemia stem cells were found to be more sensitive to cantharidin then cytarabine or daunorubicin at each of the concentrations tested. FIG. 6 shows a dose response curve of leukemia stem cells treated with cantharidin and standard chemotherapy (Ara-C and Daunorubicin) as measured by the Cobblestone Area Forming Cell Assay (CAFC).

### 7. EQUIVALENTS

The present invention is not to be limited in scope by the specific embodiments described which are intended as single illustrations of individual aspects of the invention, and functionally equivalent methods and components are within the scope of the invention. Indeed, various modifications of the invention, in addition to those shown and described herein, will become apparent to those skilled in the art from the foregoing description and accompanying drawings using no more than routine experimentation. Such modifications and equivalents are intended to fall within the scope of the appended claims.

All publications, patents and patent applications mentioned in this specification are herein incorporated by reference into the specification to the same extent as if each individual publication, patent or patent application was specifically and individually indicated to be incorporated herein by reference.

Citation or discussion of a reference herein shall not be construed as an admission that such is prior art to the present invention.

What is claimed is:

1. A method of treating cancer in a human patient, comprising administering to a human patient in need thereof a therapeutically effective regimen, the regimen comprising administering an effective amount of a compound of formula I, II, or III 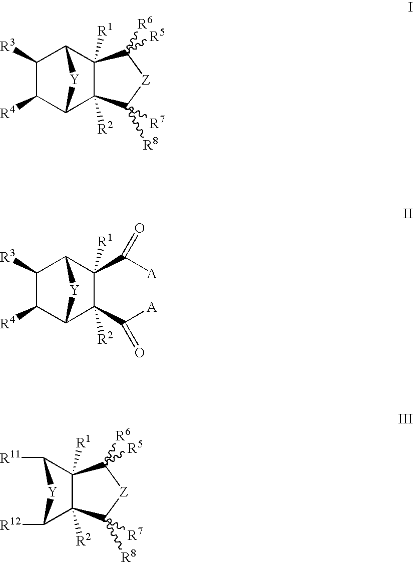
wherein
R¹ and R² are independently H or CH₃;
R³ and R⁴ are independently H, C₁-C₆ alkyl, aryl, or ara (C₁-C₁₀)alkyl; or together R³ and R⁴ form a bond (i.e., to form a cyclohexenyl ring);
R⁵, R⁶, R⁷, and R⁸ are independently H or OH, or R⁵ and R⁶, or R⁷ and R⁸ together with the carbon to which they are attached, form C═O;
R¹¹ and R¹² are independently H, C₁-C₁₀ alkyl, aryl, or ara(C₁-C₁₀)alkyl;
Y is O, N, or S;
A is OH or OR¹⁰, wherein R¹⁰ is C₁-C₆ alkyl;
Z is O, S, SR¹⁴, N—R⁹, CH₂OR¹², CHQ, or an amino acid;
R¹⁴ is C₁-C₁₀ alkyl, aryl, or ara(C₁-C₁₀)alkyl;
R⁹ is C₁-C₁₀ alkyl, H, OH, or Q;
R¹² is H or C₁-C₁₀ alkyl, aryl, or ara(C₁-C₁₀)alkyl;
wherein when Z is an amino acid, the α-amino group is a ring atom in a five-membered ring of formula I or III;
wherein Q is H or a moiety having the formula 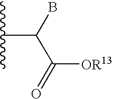
wherein R¹³ is C₁-C₁₀ alkyl or H; and B is (CH₂)ₙW, CH═CH—W, or CH₂OW, wherein W is an ionisable residue; or
a pharmaceutically acceptable salt thereof,
to the human patient, wherein the human patient has been diagnosed with cancer, and wherein said cancer is a hematological cancer.

2. The method of claim 1, wherein the regimen comprises the administration of the compound of formula I, II or III over a period of 1 to 12 months.

3. The method of claim 1, wherein the regimen results in a reduction in the amount of cancer cells.

4. The method of claim 1, wherein the method further comprises monitoring the amount of cancer cells.

5. The method of claim 4, wherein said monitoring comprises detecting in a specimen from said patient the amount of cancer cells in said specimen.

6. The method of claim 5, wherein said specimen is a blood specimen, a bone marrow sample, a normal tissue biopsy, or a tumor biopsy.

7. The method of claim 4, wherein the regimen further comprises administering an additional effective amount of the compound of formula I, II or III to the human patient subsequent to monitoring.

8. The method of claim 1, wherein the regimen results in a reduction in the amount of cancer stem cells.

9. The method of claim 1, wherein the method further comprises monitoring the amount of cancer stem cells.

10. The method of claim 9, wherein said monitoring comprises detecting in a specimen from said patient the amount of cancer stem cells in said specimen.

11. The method of claim 10, wherein said specimen is a blood specimen, a bone marrow sample, a normal tissue biopsy, or a tumor biopsy.

12. The method of claim 1, wherein the regimen comprises intravenous or subcutaneous administration of the compound of formula I, II or III.

13. The method of claim 12, wherein the regimen comprises intravenous administration in a dose of 50 mg/kg or less.

14. The method of claim 12, wherein the regimen comprises subcutaneous administration in a dose of 50 mg/kg or less.

15. The method of claim 1, wherein the regimen further comprises the administration of an additional therapy, and wherein the compound of formula I, II or III and the additional therapy are administered separately, concurrently, or sequentially.

16. The method of claim 16, wherein the additional therapy is chemotherapy, small molecule therapy, radioimmunotherapy, toxin therapy, prodrug-activating enzyme therapy, antibody therapy, surgical therapy, immunotherapy, anti-angiogenic therapy, targeted therapy, radiation therapy, biologic therapy, epigenetic therapy, hormonal therapy, differentiation therapy or any combination thereof.

17. The method of claim 1, wherein the patient has received a therapy for the treatment of cancer prior to the administration of the therapeutically effective regimen of the compound of formula I, II or III.

18. The method of claim 17, wherein the therapy is chemotherapy, small molecule therapy, radioimmunotherapy, toxin therapy, prodrug-activating enzyme therapy, antibody therapy, surgical therapy, immunotherapy, anti-angiogenic therapy, targeted therapy, radiation therapy, biologic therapy, epigenetic therapy, hormonal therapy, differentiation therapy or any combination thereof.

19. The method of claim 1, wherein the compound is of formula I,
R¹ and R² are both CH₃, R³ and R⁴ are both H,
R⁵ and R⁶ together with the carbon to which they are attached form C═O,
R⁷ and R⁸ together with the carbon to which they are attached form C═O, and
Y and Z are both O.

20. The method of claim 19, wherein the compound of formula I is administered to the human patient at a dose ranging from 0.1 to 25 mg/kg.

21. The method of claim 1, wherein the compound is of formula I,
R¹, R², R³, and R⁴ are H,
R⁵ and R⁶ together with the carbon to which they are attached form C═O,
R⁷ and R⁸ together with the carbon to which they are attached form C═O, and
Y and Z are both O.

22. The method of claim 21, wherein the compound of formula I is administered to the human patient at a dose ranging from 0.1 to 25 mg/kg.

23. The method of claim 1, wherein the compound is of formula II,
R¹ and R² are CH₃, R³ and R⁴ are H,
Y is O, and
A is OH;
or a pharmaceutically acceptable salt thereof.

24. The method of claim 23, wherein the compound is disodium cantharidate.

25. The method of claim 24, wherein the compound is administered to the human patient at a dose ranging from 0.1 to 25 mg/kg.

26. The method of claim 1, wherein the regimen comprises the administration of the compound of formula I, II or III in combination with an additional therapy and, wherein the compound of formula I, II or III and the additional therapy are administered separately, concurrently, or sequentially.

27. A method of treating cancer in a human patient, comprising administering to a human patient in need thereof a therapeutically effective regimen, the regimen comprising administering an effective amount of a compound of formula I, II or III 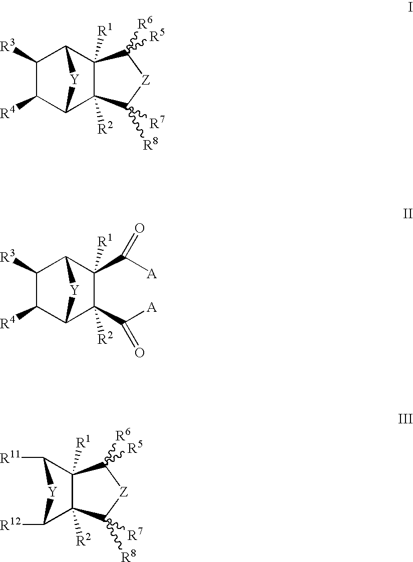
wherein
R¹ and R² are independently H or CH₃;
R³ and R⁴ are independently H, C₁-C₆ alkyl, aryl, or ara (C₁-C₁₀)alkyl; or together R³ and R⁴ form a bond (i.e., to form a cyclohexenyl ring);
R⁵, R⁶, R⁷, and R⁸ are independently H or OH, or R⁵ and R⁶, or R⁷ and R⁸ together with the carbon to which they are attached, form C═O;
R¹¹ and R¹² are independently H, C₁-C₁₀ alkyl, aryl, or ara(C₁-C₁₀)alkyl;
Y is O, N, or S;
A is OH or OR¹⁰, wherein R¹⁰ is C₁-C₆ alkyl;
Z is O, S, SR¹⁴, N—R⁹, CH₂OR¹², CHQ, or an amino acid;
R¹⁴ is C₁-C₁₀ alkyl, aryl, or ara(C₁-C₁₀)alkyl;
R⁹ is C₁-C₁₀ alkyl, H, OH, or Q;
R¹² is H or C₁-C₁₀ alkyl, aryl, or ara(C₁-C₁₀)alkyl;
wherein when Z is an amino acid, the α-amino group is a ring atom in a five-membered ring of formula I or III;
wherein Q is H or a moiety having the formula 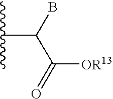
wherein R¹³ is C₁-C₁₀ alkyl or H; and B is (CH₂)ₙW, CH═CH—W, or CH₂OW, wherein W is an ionisable residue; or
a pharmaceutically acceptable salt thereof,
to the patient, wherein the human patient has been diagnosed with cancer, wherein said cancer is a hematological cancer, and wherein the patient has not previously received therapy for said cancer.

28. The method of claim 27, wherein the regimen comprises the administration of the compound of formula I, II or III over a period of 1 to 12 months.

29. The method of claim 27, wherein the regimen results in a reduction in the amount of cancer cells.

30. The method of claim 27, wherein the method further comprises monitoring the amount of cancer cells.

31. The method of claim 30, wherein said monitoring comprises detecting in a specimen from said patient the amount of cancer cells in said specimen.

32. The method of claim 31, wherein said specimen is a blood specimen, a bone marrow sample, a normal tissue biopsy, or a tumor biopsy.

33. The method of claim 27, wherein the regimen results in a reduction in the amount of cancer stem cells.

34. The method of claim 27, wherein the method further comprises monitoring the amount of cancer stem cells.

35. The method of claim 34, wherein said monitoring comprises detecting in a specimen from said patient the amount of cancer stem cells in said specimen.

36. The method of claim 35, wherein said specimen is a blood specimen, a bone marrow sample, a normal tissue biopsy or a tumor biopsy.

37. The method of claim 27, wherein the regimen comprises intravenous or subcutaneous administration of the compound of formula I, II or III.

38. The method of claim 37, wherein the regimen comprises intravenous administration in a dose of 50 mg/kg or less.

39. The method of claim 37, wherein the regimen comprises subcutaneous administration in a dose of 50 mg/kg or less.

40. The method of claim 27, wherein the compound is of formula I,
R¹ and R² are both CH₃, R³ and R⁴ are both H,
R⁵ and R⁶ together with the carbon to which they are attached form C═O,
R⁷ and R⁸ together with the carbon to which they are attached form C═O, and
Y and Z are both O.

41. The method of claim 40, wherein the compound of formula I is administered to the human patient at a dose ranging from 0.1 to 25 mg/kg.

42. The method of claim 27, wherein the compound is of formula I,
R¹, R², R³, and R⁴ are H,
R⁵ and R⁶ together with the carbon to which they are attached form C═O,
R⁷ and R⁸ together with the carbon to which they are attached form C═O, and
Y and Z are both O.

43. The method of claim 42, wherein the compound of formula I is administered to the human patient at a dose ranging from 0.1 to 25 mg/kg.

44. The method of claim 27, wherein the compound is of formula II,
R¹ and R² are CH₃, R³ and R⁴ are H,
Y is O, and
A is OH;
or a pharmaceutically acceptable salt thereof.

45. The method of claim 44, wherein the compound is disodium cantharidate.

46. The method of claim 45, wherein the compound is administered to the human patient at a dose ranging from 0.1 to 25 mg/kg.

47. The method of claim 27, wherein the regimen further comprises the administration of an additional therapy, and wherein the compound of formula I, II or III and the additional therapy are administered separately, concurrently, or sequentially.

48. The method of claim 47, wherein the additional therapy is chemotherapy, small molecule therapy, radioimmunotherapy, toxin therapy, prodrug-activating enzyme therapy, antibody therapy, surgical therapy, immunotherapy, anti-angiogenic therapy, targeted therapy, radiation therapy, biological therapy, epigenetic therapy, hormonal therapy, differentiation therapy or any combination thereof.

49. The method of claim 27, wherein the regimen comprises the administration of the compound of formula I, II or III in combination with an additional therapy, wherein the compound of formula I, II or III and the additional therapy are administered separately, concurrently, or sequentially.

50. A method of treating a solid tumor in a human patient, comprising administering to a human patient in need thereof a therapeutically effective regimen, the regimen comprising administering an effective amount of a compound of formula I, II or III 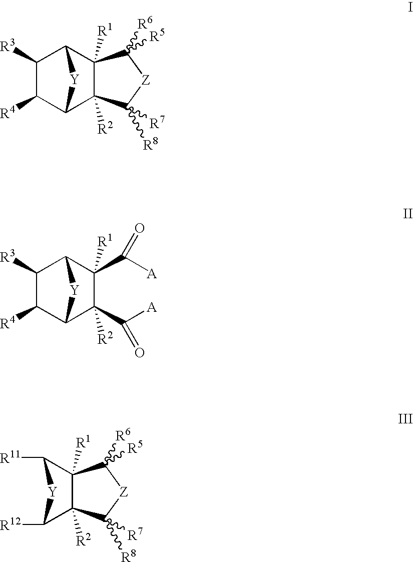
wherein
R¹ and R² are independently H or CH₃;
R³ and R⁴ are independently H, C₁-C₆ alkyl, aryl, or ara (C₁-C₁₀)alkyl; or together R³ and R⁴ form a bond (i.e., to form a cyclohexenyl ring);
R⁵, R⁶, R⁷, and R⁸ are independently H or OH, or R⁵ and R⁶, or R⁷ and R⁸ together with the carbon to which they are attached, form C═O;
R¹¹ and R¹² are independently H, C₁-C₁₀ alkyl, aryl, or ara(C₁-C₁₀)alkyl;
Y is O, N, or S;
A is OH or OR¹⁰, wherein R¹⁰ is C₁-C₆ alkyl;
Z is O, S, SR¹⁴, N—R⁹, CH₂OR¹², CHQ, or an amino acid;
R¹⁴ is C₁-C₁₀ alkyl, aryl, or ara(C₁-C₁₀)alkyl;
R⁹ is C₁-C₁₀ alkyl, H, OH, or Q;
R¹² is H or C₁-C₁₀ alkyl, aryl, or ara(C₁-C₁₀)alkyl;
wherein when Z is an amino acid, the α-amino group is a ring atom in a five-membered ring of formula I or III;
wherein Q is H or a moiety having the formula 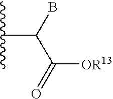
wherein R¹³ is C₁-C₁₀ alkyl or H; and B is (CH₂)ₙW, CH═CH—
W, or CH₂OW, wherein W is an ionisable residue; or
a pharmaceutically acceptable salt thereof,
to the patient, wherein the human patient has been diagnosed with a solid tumor, and wherein the patient has undergone prior therapy for cancer.

51. The method of claim 50, wherein said solid tumor is fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, melanoma, neuroblastoma, or retinoblastoma.

52. A method of treating cancer in a human patient, comprising administering to a human patient in need thereof a therapeutically effective regimen, the regimen comprising administering an effective amount of a compound of formula I, II or III 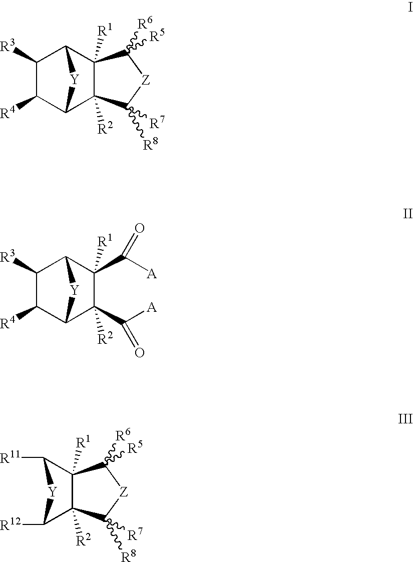
wherein
R¹ and R² are independently H or CH₃;
R³ and R⁴ are independently H, C₁-C₆ alkyl, aryl, or ara (C₁-C₁₀)alkyl; or together R³ and R⁴ form a bond (i.e., to form a cyclohexenyl ring);
R⁵, R⁶, R⁷, and R⁸ are independently H or OH, or R⁵ and R⁶, or R⁷ and R⁸ together with the carbon to which they are attached, form C═O;
R¹¹ and R¹² are independently H, C₁-C₁₀ alkyl, aryl, or ara(C₁-C₁₀)alkyl;
Y is O, N, or S;
A is OH or OR¹⁰, wherein R¹⁰ is C₁-C₆ alkyl;
Z is O, S, SR¹⁴, N—R⁹, CH₂OR¹², CHQ, or an amino acid;
R¹⁴ is C₁-C₁₀ alkyl, aryl, or ara(C₁-C₁₀)alkyl;
R⁹ is C₁-C₁₀ alkyl, H, OH, or Q;
R¹² is H or C₁-C₁₀ alkyl, aryl, or ara(C₁-C₁₀)alkyl;
wherein when Z is an amino acid, the α-amino group is a ring atom in a five-membered ring of formula I or III;
wherein Q is H or a moiety having the formula 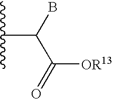
wherein R¹³ is C₁-C₁₀ alkyl or H; and B is (CH₂)ₙW, CH═CH—
W, or CH₂OW, wherein W is an ionisable residue; or
a pharmaceutically acceptable salt thereof,
to the patient, wherein the human patient has undergone prior therapy for cancer.

53. A method of preventing cancer in a human patient, comprising administering to a human subject in need thereof a prophylactically effective regimen of a compound of formula I, II or III 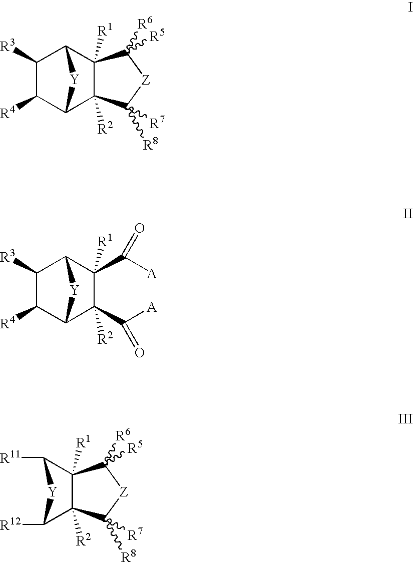
wherein
R¹ and R² are independently H or CH₃;
R³ and R⁴ are independently H, C₁-C₆ alkyl, aryl, or ara (C₁-C₁₀)alkyl; or together R³ and R⁴ form a bond (i.e., to form a cyclohexenyl ring);
R⁵, R⁶, R⁷, and R⁸ are independently H or OH, OR⁵ and R⁶, or R⁷ and R⁸ together with the carbon to which they are attached, form C═O;
R¹¹ and R¹² are independently H, C₁-C₁₀ alkyl, aryl, or ara(C₁-C₁₀)alkyl;
Y is O, N, or S;
A is OH or OR¹⁰, wherein R¹⁰ is C₁-C₆ alkyl;
Z is O, S, SR¹⁴, N—R⁹, CH₂OR¹², CHQ, or an amino acid;
R¹⁴ is C₁-C₁₀ alkyl, aryl, or ara(C₁-C₁₀)alkyl;
R⁹ is C₁-C₁₀ alkyl, H, OH, or Q;
R¹² is H or C₁-C₁₀ alkyl, aryl, or ara(C₁-C₁₀)alkyl;
wherein when Z is an amino acid, the α-amino group is a ring atom in a five-membered ring of formula I or III;
wherein Q is H or a moiety having the formula 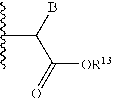
wherein R¹³ is C₁-C₁₀ alkyl or H; and B is (CH₂)ₙW, CH═CH—W, or CH₂OW, wherein W is an ionisable residue; or
a pharmaceutically acceptable salt thereof,
to the patient, wherein the human patient is in remission from the cancer.

54. A method of treating kidney cancer in a human patient comprising administering to a human patient in need thereof a therapeutically effective regimen, the regimen comprising administering a compound of formula I, II or III 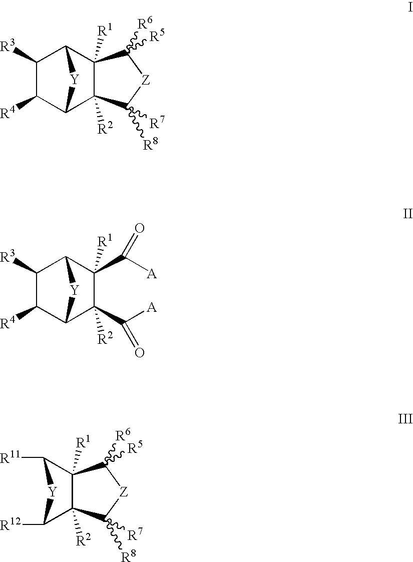
wherein
R¹ and R² are independently H or CH₃;
R³ and R⁴ are independently H, C₁-C₆ alkyl, aryl, or ara (C₁-C₁₀)alkyl; or together R³ and R⁴ form a bond (i.e., to form a cyclohexenyl ring);
R⁵, R⁶, R⁷, and R⁸ are independently H or OH, or R⁵ and R⁶, or R⁷ and R⁸ together with the carbon to which they are attached, form C═O;
R¹¹ and R¹² are independently H, C₁-C₁₀ alkyl, aryl, or ara(C₁-C₁₀)alkyl;
Y is O, N, or S;
A is OH or OR¹⁰, wherein R¹⁰ is C₁-C₆ alkyl;
Z is O, S, SR¹⁴, N—R⁹, CH₂OR¹², CHQ, or an amino acid;
R¹⁴ is C₁-C₁₀ alkyl, aryl, or ara(C₁-C₁₀)alkyl;
R⁹ is C₁-C₁₀ alkyl, H, OH, or Q;
R¹² is H or C₁-C₁₀ alkyl, aryl, or ara(C₁-C₁₀)alkyl;
wherein when Z is an amino acid, the α-amino group is a ring atom in a five-membered ring of formula I or III;
wherein Q is H or a moiety having the formula 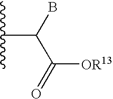
wherein R¹³ is C₁-C₁₀ alkyl or H; and B is (CH₂)ₙW, CH═CH—W, or CH₂OW, wherein W is an ionisable residue; or
a pharmaceutically acceptable salt thereof,
to the patient, wherein the patient has been diagnosed with kidney cancer.

55. A method of treating pancreatic cancer in a human patient, comprising administering to a human patient in need thereof a therapeutically effective regimen, the regimen comprising administering of a compound of formula I, II or III 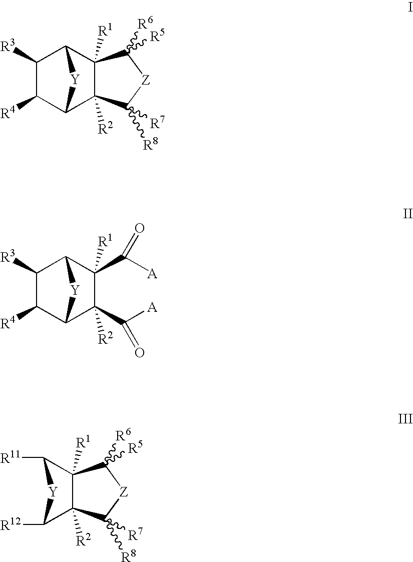
wherein
R¹ and R² are independently H or CH₃;
R³ and R⁴ are independently H, C₁-C₆ alkyl, aryl, or ara (C₁-C₁₀)alkyl; or together R³ and R⁴ form a bond (i.e., to form a cyclohexenyl ring);
R⁵, R⁶, R⁷, and R⁸ are independently H or OH, or R⁵ and R⁶, or R⁷ and R⁸ together with the carbon to which they are attached, form C═O;
R¹ and R² are independently H, C₁-C₁₀ alkyl, aryl, or ara(C₁-C₁₀)alkyl;
Y is O, N, or S;
A is OH or OR¹⁰, wherein R¹⁰ is C₁-C₆ alkyl;
Z is O, S, SR¹⁴, N—R⁹, CH₂OR², CHQ, or an amino acid;
R¹⁴ is C₁-C₁₀ alkyl, aryl, or ara(C₁-C₁₀)alkyl;
R⁹ is C₁-C₁₀ alkyl, H, OH, or Q;
R¹² is H or C₁-C₁₀ alkyl, aryl, or ara(C₁-C₁₀)alkyl;
wherein when Z is an amino acid, the α-amino group is a ring atom in a five-membered ring of formula I or III;
wherein Q is H or a moiety having the formula 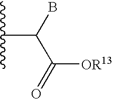
wherein R¹³ is C₁-C₁₀ alkyl or H; and B is (CH₂)ₙW, CH═CH—W, or CH₂OW, wherein W is an ionisable residue; or
a pharmaceutically acceptable salt thereof,
to the patient, wherein the patient has been diagnosed with pancreatic cancer.

56. A method of treating bone cancer in a human patient, comprising administering to a human patient in need thereof a therapeutically effective regimen, the regimen comprising administering a compound of formula I, II or III 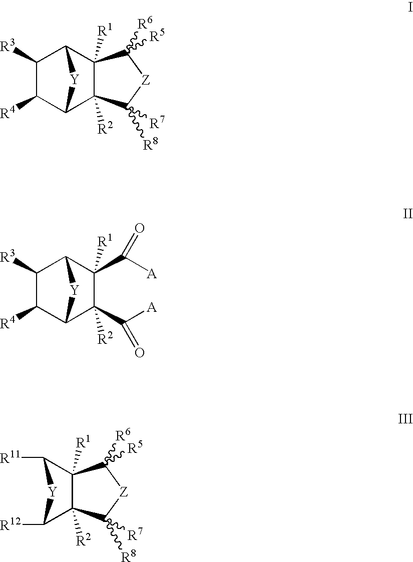
wherein
R¹ and R² are independently H or CH₃;
R³ and R⁴ are independently H, C₁-C₆ alkyl, aryl, or ara (C₁-C₁₀)alkyl; or together R³ and R⁴ form a bond (i.e., to form a cyclohexenyl ring);
R⁵, R⁶, R⁷, and R⁸ are independently H or OH, or R⁵ and R⁶, or R⁷ and R⁸ together with the carbon to which they are attached, form C═O;
R¹¹ and R¹² are independently H, C₁-C₁₀ alkyl, aryl, or ara(C₁-C₁₀)alkyl;
Y is O, N, or S;
A is OH or OR¹⁰, wherein R¹⁰ is C₁-C₆ alkyl;
Z is O, S, SR¹⁴, N—R⁹, CH₂OR¹², CHQ, or an amino acid;
R¹⁴ is C₁-C₁₀ alkyl, aryl, or ara(C₁-C₁₀)alkyl;
R⁹ is C₁-C₁₀ alkyl, H, OH, or Q;
R¹² is H or C₁-C₁₀ alkyl, aryl, or ara(C₁-C₁₀)alkyl;
wherein when Z is an amino acid, the α-amino group is a ring atom in a five-membered ring of formula I or III;
wherein Q is H or a moiety having the formula 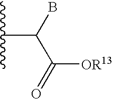
wherein R¹³ is C₁-C₁₀ alkyl or H; and B is (CH₂)ₙW, CH═CH—W, or CH₂OW, wherein W is an ionisable residue; or
a pharmaceutically acceptable salt thereof,
to the patient, wherein the patient has been diagnosed with bone cancer.

57. A method of treating breast cancer in a human patient, comprising administering to a human patient in need thereof a therapeutically effective regimen, the regimen comprising administering a compound of formula I, II or III 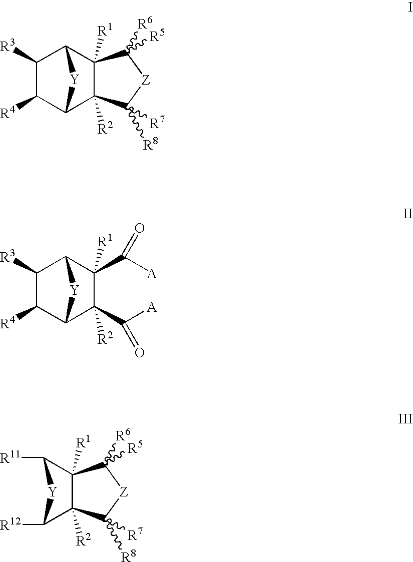
wherein
R¹ and R² are independently H or CH₃;
R³ and R⁴ are independently H, C₁-C₆ alkyl, aryl, or ara (C₁-C₁₀)alkyl; or together R³ and R⁴ form a bond (i.e., to form a cyclohexenyl ring);
R⁵, R⁶, R⁷, and R⁸ are independently H or OH, or R⁵ and R⁶, or R⁷ and R⁸ together with the carbon to which they are attached, form C═O;
R¹¹ and R¹² are independently H, C₁-C₁₀ alkyl, aryl, or ara(C₁-C₁₀)alkyl;
Y is O, N, or S;
A is OH or OR¹⁰, wherein R¹⁰ is C₁-C₆ alkyl;
Z is O, S, SR¹⁴, N—R⁹, CH₂OR¹², CHQ, or an amino acid;
R¹⁴ is C₁-C₁₀ alkyl, aryl, or ara(C₁-C₁₀)alkyl;
R⁹ is C₁-C₁₀ alkyl, H, OH, or Q;
R¹² is H or C₁-C₁₀ alkyl, aryl, or ara(C₁-C₁₀)alkyl;
wherein when Z is an amino acid, the α-amino group is a ring atom in a five-membered ring of formula I or III;
wherein Q is H or a moiety having the formula 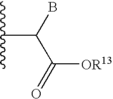
wherein R¹³ is C₁-C₁₀ alkyl or H; and B is (CH₂)ₙW, CH═CH—W, or CH₂OW, wherein W is an ionisable residue; or
a pharmaceutically acceptable salt thereof,
to the patient, wherein the patient has been diagnosed with breast cancer.

58. A method of treating ovarian cancer in a human patient, comprising administering to a human subject in need thereof a therapeutically effective regimen, the regimen comprising administering a compound of formula I, II or III 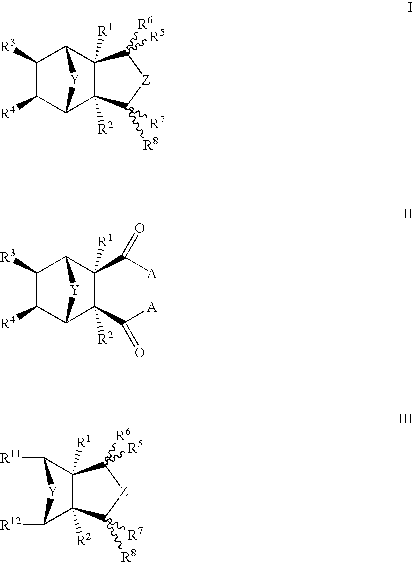
wherein
R¹ and R² are independently H or CH₃;
R³ and R⁴ are independently H, C₁-C₆ alkyl, aryl, or ara (C₁-C₁₀)alkyl; or together R³ and R⁴ form a bond (i.e., to form a cyclohexenyl ring);
R⁵, R⁶, R⁷, and R⁸ are independently H or OH, or R⁵ and R⁶, or R⁷ and R⁸ together with the carbon to which they are attached, form C═O;
R¹¹ and R¹² are independently H, C₁-C₁₀ alkyl, aryl, or ara(C₁-C₁₀)alkyl;
Y is O, N, or S;
A is OH or OR¹⁰, wherein R¹⁰ is C₁-C₆ alkyl;
Z is O, S, SR¹⁴, N—R⁹, CH₂OR¹², CHQ, or an amino acid;
R¹⁴ is C₁-C₁₀ alkyl, aryl, or ara(C₁-C₁₀)alkyl;
R⁹ is C₁-C₁₀ alkyl, H, OH, or Q;
R¹² is H or C₁-C₁₀ alkyl, aryl, or ara(C₁-C₁₀)alkyl;
wherein when Z is an amino acid, the α-amino group is a ring atom in a five-membered ring of formula I or III;
wherein Q is H or a moiety having the formula 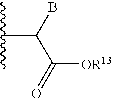
wherein R¹³ is C₁-C₁₀ alkyl or H; and B is (CH₂)ₙW, CH═CH—W, or CH₂OW, wherein W is an ionisable residue; or
a pharmaceutically acceptable salt thereof,
to the patient, wherein the patient has been diagnosed with ovarian cancer.

59. A method of treating prostate cancer in a human patient, comprising administering to a human subject in need thereof a therapeutically effective regimen, the regimen comprising administering a compound of formula I, II or III 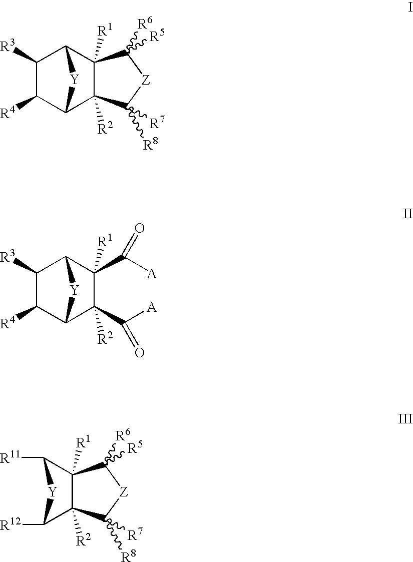
wherein
R¹ and R² are independently H or CH₃;
R³ and R⁴ are independently H, C₁-C₆ alkyl, aryl, or ara (C₁-C₁₀)alkyl; or together R³ and R⁴ form a bond (i.e., to form a cyclohexenyl ring);
R⁵, R⁶, R⁷, and R⁸ are independently H or OH, or R⁵ and R⁶, or R⁷ and R⁸ together with the carbon to which they are attached, form C═O;
R¹¹ and R¹² are independently H, C₁-C₁₀ alkyl, aryl, or ara(C₁-C₁₀)alkyl;
Y is O, N, or S;
A is OH or OR¹⁰, wherein R¹⁰ is C₁-C₆ alkyl;
Z is O, S, SR¹⁴, N—R⁹, CH₂OR², CHQ, or an amino acid;
R¹⁴ is C₁-C₁₀ alkyl, aryl, or ara(C₁-C₁₀)alkyl;
R⁹ is C₁-C₁₀alkyl, H, OH, or Q;
R¹² is H or C₁-C₁₀ alkyl, aryl, or ara(C₁-C₁₀)alkyl;
wherein when Z is an amino acid, the α-amino group is a ring atom in a five-membered ring of formula I or III;
wherein Q is H or a moiety having the formula 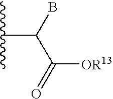
wherein R¹³ is C₁-C₁₀ alkyl or H; and B is (CH₂)ₙW, CH═CH—W, or CH₂OW, wherein W is an ionisable residue; or
a pharmaceutically acceptable salt thereof,
to the patient, wherein the patient has been diagnosed with prostate cancer.

60. A method of treating cervical cancer in a human patient, comprising administering to a human patient in need thereof a therapeutically effective regimen, the regimen comprising administering a compound of formula I, II or III 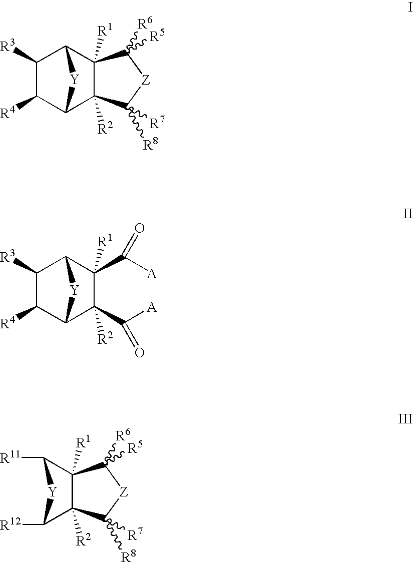
wherein
R¹ and R² are independently H or CH₃;
R³ and R⁴ are independently H, C₁-C₆ alkyl, aryl, or ara (C₁-C₁₀)alkyl; or together R³ and R⁴ form a bond (i.e., to form a cyclohexenyl ring);
R⁵, R⁶, R⁷, and R⁸ are independently H or OH, or R⁵ and R⁶, or R⁷ and R⁸ together with the carbon to which they are attached, form C═O;
R¹¹ and R¹² are independently H, C₁-C₁₀ alkyl, aryl, or ara(C₁-C₁₀)alkyl;
Y is O, N, or S;
A is OH or OR¹⁰, wherein R¹⁰ is C₁-C₆ alkyl;
Z is O, S, SR¹⁴, N—R⁹, CH₂OR¹², CHQ, or an amino acid;
R¹⁴ is C₁-C₁₀ alkyl, aryl, or ara(C₁-C₁₀)alkyl;
R⁹ is C₁-C₁₀ alkyl, H, OH, or Q;
R¹² is H or C₁-C₁₀ alkyl, aryl, or ara(C₁-C₁₀)alkyl;
wherein when Z is an amino acid, the α-amino group is a ring atom in a five-membered ring of formula I or III;
wherein Q is H or a moiety having the formula 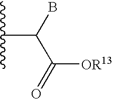
wherein R¹³ is C₁-C₁₀ alkyl or H; and B is (CH₂)ₙW, CH═CH—W, or CH₂OW, wherein W is an ionisable residue; or
a pharmaceutically acceptable salt thereof,
to the patient, wherein the patient has been diagnosed with cervical cancer.

61. A method of treating uterine cancer in a human patient, comprising administering to a human patient in need thereof a therapeutically effective regimen, the regimen comprising administering a compound of formula I, II or III 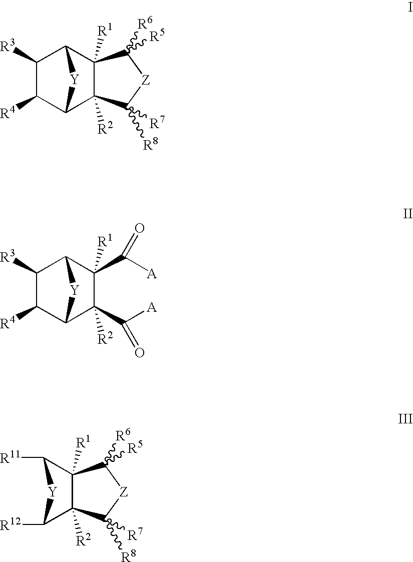
wherein
R¹ and R² are independently H or CH₃;
R³ and R⁴ are independently H, C₁-C₆ alkyl, aryl, or ara (C₁-C₁₀)alkyl; or
together R³ and R⁴ form a bond (i.e., to form a cyclohexenyl ring);
R⁵, R⁶, R⁷, and R⁸ are independently H or OH, or R⁵ and R⁶, or R⁷ and R⁸ together with the carbon to which they are attached, form C═O;
R¹¹ and R¹² are independently H, C₁-C₁₀ alkyl, aryl, or ara(C₁-C₁₀)alkyl;
Y is O, N, or S;
A is OH or OR¹⁰, wherein R¹⁰ is C₁-C₆ alkyl;
Z is O, S, SR¹⁴, N—R⁹, CH₂OR¹², CHQ, or an amino acid;
R¹⁴ is C₁-C₁₀ alkyl, aryl, or ara(C₁-C₁₀)alkyl;
R⁹ is C₁-C₁₀ alkyl, H, OH, or Q;
R¹² is H or C₁-C₁₀ alkyl, aryl, or ara(C₁-C₁₀)alkyl;
wherein when Z is an amino acid, the α-amino group is a ring atom in a five-membered ring of formula I or III;
wherein Q is H or a moiety having the formula 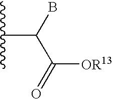
wherein R¹³ is C₁-C₁₀ alkyl or H; and B is (CH₂)ₙW, CH═CH—W, or CH₂OW, wherein W is an ionisable residue; or
a pharmaceutically acceptable salt thereof,
to the patient, wherein the patient has been diagnosed with uterine cancer.

62. A method of treating testicular cancer in a human patient, comprising administering to a human patient in need thereof a therapeutically effective regimen, the regimen comprising a compound of formula I, II or III 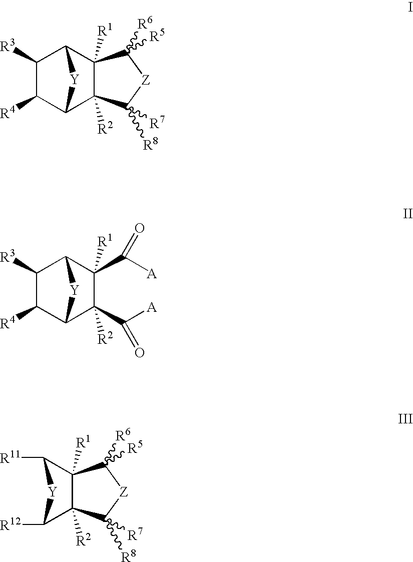
wherein
R¹ and R² are independently H or CH₃;
R³ and R⁴ are independently H, C₁-C₆ alkyl, aryl, or ara (C₁-C₁₀)alkyl; or together R³ and R⁴ form a bond (i.e., to form a cyclohexenyl ring);
R⁵, R⁶, R⁷, and R⁸ are independently H or OH, or R⁵ and R⁶, or R⁷ and R⁸ together with the carbon to which they are attached, form C═O;
R¹¹ and R¹² are independently H, C₁-C₁₀ alkyl, aryl, or ara(C₁-C₁₀)alkyl;
Y is O, N, or S;
A is OH or OR¹⁰, wherein R¹⁰ is C₁-C₆ alkyl;
Z is O, S, SR¹⁴, N—R⁹, CH₂OR¹², CHQ, or an amino acid;
R¹⁴ is C₁-C₁₀ alkyl, aryl, or ara(C₁-C₁₀)alkyl;
R⁹ is C₁-C₁₀ alkyl, H, OH, or Q;
R¹² is H or C₁-C₁₀ alkyl, aryl, or ara(C₁-C₁₀)alkyl;
wherein when Z is an amino acid, the α-amino group is a ring atom in a five-membered ring of formula I or III;
wherein Q is H or a moiety having the formula 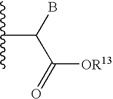
wherein R¹³ is C₁-C₁₀ alkyl or H; and B is (CH₂)ₙW, CH═CH—W, or CH₂OW, wherein W is an ionisable residue; or
a pharmaceutically acceptable salt thereof,
to the patient, wherein the patient has been diagnosed with testicular cancer.

63. A method of treating bladder cancer in a human patient, comprising administering to a human patient in need thereof a therapeutically effective regimen, the regimen comprising administering a compound of formula I, II or III 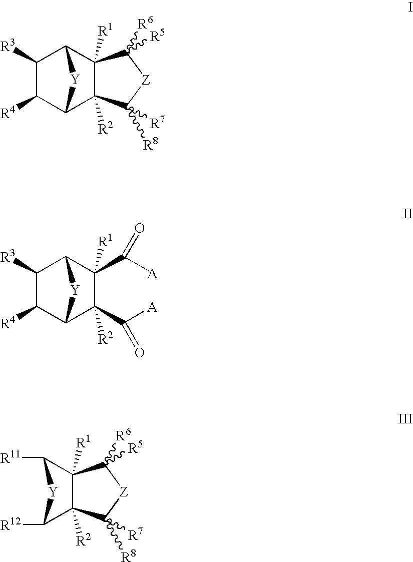
wherein
R¹ and R² are independently H or CH₃;
R³ and R⁴ are independently H, C₁-C₆ alkyl, aryl, or ara (C₁-C₁₀)alkyl; or together R³ and R⁴ form a bond (i.e., to form a cyclohexenyl ring);
R⁵, R⁶, R⁷, and R⁸ are independently H or OH, or R⁵ and R⁶, or R⁷ and R⁸ together with the carbon to which they are attached, form C═O;
R¹¹ and R¹² are independently H, C₁-C₁₀ alkyl, aryl, or ara(C₁-C₁₀)alkyl;
Y is O, N, or S;
A is OH or OR¹⁰, wherein R¹⁰ is C₁-C₆ alkyl;
Z is O, S, SR¹⁴, N—R⁹, CH₂OR¹², CHQ, or an amino acid;
R¹⁴ is C₁-C₁₀ alkyl, aryl, or ara(C₁-C₁₀)alkyl;
R⁹ is C₁-C₁₀ alkyl, H, OH, or Q;
R¹² is H or C₁-C₁₀ alkyl, aryl, or ara(C₁-C₁₀)alkyl;
wherein when Z is an amino acid, the α-amino group is a ring atom in a five-membered ring of formula I or III;
wherein Q is H or a moiety having the formula 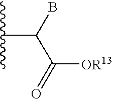
wherein R¹³ is C₁-C₁₀ alkyl or H; and B is (CH₂)ₙW, CH═CH—W, or CH₂OW, wherein W is an ionisable residue; or
a pharmaceutically acceptable salt thereof,
to the patient, wherein the patient has been diagnosed with bladder cancer.

64. A method of treating skin cancer in a human patient, comprising administering to a human patient in need thereof a therapeutically effective regimen, the regimen comprising administering of a compound of formula I, II or III 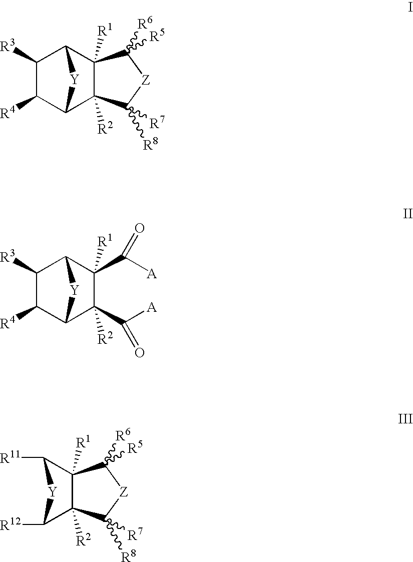
wherein
R¹ and R² are independently H or CH₃;
R³ and R⁴ are independently H, C₁-C₆ alkyl, aryl, or ara (C₁-C₁₀)alkyl; or together R³ and R⁴ form a bond (i.e., to form a cyclohexenyl ring);
R⁵, R⁶, R⁷, and R⁸ are independently H or OH, or R⁵ and R⁶, or R⁷ and R⁸ together with the carbon to which they are attached, form C═O;
R¹¹ and R¹² are independently H, C₁-C₁₀ alkyl, aryl, or ara(C₁-C₁₀)alkyl;
Y is O, N, or S;
A is OH or OR¹⁰, wherein R¹⁰ is C₁-C₆ alkyl;
Z is O, S, SR¹⁴, N—R⁹, CH₂OR¹², CHQ, or an amino acid;
R¹⁴ is C₁-C₁₀ alkyl, aryl, or ara(C₁-C₁₀)alkyl;
R⁹ is C₁-C₁₀ alkyl, H, OH, or Q;
R¹² is H or C₁-C₁₀ alkyl, aryl, or ara(C₁-C₁₀)alkyl;
wherein when Z is an amino acid, the α-amino group is a ring atom in a five-membered ring of formula I or III;
wherein Q is H or a moiety having the formula 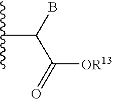
wherein R¹³ is C₁-C₁₀ alkyl or H; and B is (CH₂)ₙW, CH═CH—W, or CH₂OW, wherein W is an ionisable residue; or
a pharmaceutically acceptable salt thereof,
to the patient, wherein the patient has been diagnosed with skin cancer.

65. A method of treating melanoma in a human patient, comprising administering to a human patient in need thereof a therapeutically effective regimen, the regimen comprising administering a compound of formula I, II or III 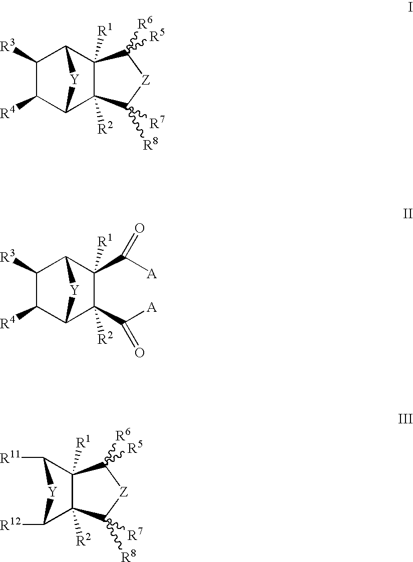
wherein
R¹ and R² are independently H or CH₃;
R³ and R⁴ are independently H, C₁-C₆ alkyl, aryl, or ara (C₁-C₁₀)alkyl; or together R³ and R⁴ form a bond (i.e., to form a cyclohexenyl ring);
R⁵, R⁶, R⁷, and R⁸ are independently H or OH, or R⁵ and R⁶, or R⁷ and R⁸ together with the carbon to which they are attached, form C═O;
R¹¹ and R¹² are independently H, C₁-C₁₀ alkyl, aryl, or ara(C₁-C₁₀)alkyl;
Y is O, N, or S;
A is OH or OR¹⁰, wherein R¹⁰ is C₁-C₆ alkyl;
Z is O, S, SR¹⁴, N—R⁹, CH₂OR¹², CHQ, or an amino acid;
R¹⁴ is C₁-C₁₀ alkyl, aryl, or ara(C₁-C₁₀)alkyl;
R⁹ is C₁-C₁₀ alkyl, H, OH, or Q;
R¹² is H or C₁-C₁₀ alkyl, aryl, or ara(C₁-C₁₀)alkyl;
wherein when Z is an amino acid, the α-amino group is a ring atom in a five-membered ring of formula I or III;
wherein Q is H or a moiety having the formula 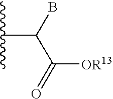
wherein R¹³ is C₁-C₁₀ alkyl or H; and B is (CH₂)ₙW, CH═CH—W, or CH₂OW, wherein W is an ionisable residue; or
a pharmaceutically acceptable salt thereof,
to the patient, wherein the patient has been diagnosed with melanoma.

66. A method of treating neuroblastoma in a human patient, comprising administering to a human subject in need thereof a therapeutically effective regimen, the regimen comprising administering a compound of formula I, II or III 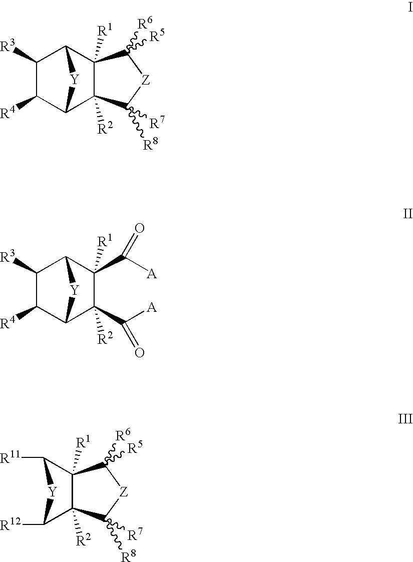
wherein
R¹ and R² are independently H or CH₃;
R³ and R⁴ are independently H, C₁-C₆ alkyl, aryl, or ara (C₁-C₁₀)alkyl; or together R³ and R⁴ form a bond (i.e., to form a cyclohexenyl ring);
R⁵, R⁶, R⁷, and R⁸ are independently H or OH, or R⁵ and R⁶, or R⁷ and R⁸ together with the carbon to which they are attached, form C═O;
R¹¹ and R¹² are independently H, C₁-C₁₀ alkyl, aryl, or ara(C₁-C₁₀)alkyl;
Y is O, N, or S;
A is OH or OR¹⁰, wherein R¹⁰ is C₁-C₆ alkyl;
Z is O, S, SR¹⁴, N—R⁹, CH₂OR¹², CHQ, or an amino acid;
R¹⁴ is C₁-C₁₀ alkyl, aryl, or ara(C₁-C₁₀)alkyl;
R⁹ is C₁-C₁₀ alkyl, H, OH, or Q;
R¹² is H or C₁-C₁₀ alkyl, aryl, or ara(C₁-C₁₀)alkyl;
wherein when Z is an amino acid, the α-amino group is a ring atom in a five-membered ring of formula I or III;
wherein Q is H or a moiety having the formula 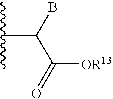
wherein R¹³ is C₁-C₁₀ alkyl or H; and B is (CH₂)ₙW, CH═CH—W, or CH₂OW, wherein W is an ionisable residue; or
a pharmaceutically acceptable salt thereof,
to the patient, wherein the patient has been diagnosed with neuroblastoma.

67. A method of treating lymphoma in a human patient, comprising administering to a human patient a therapeutically effective regimen, the regimen comprising administering a compound of formula I, II or III 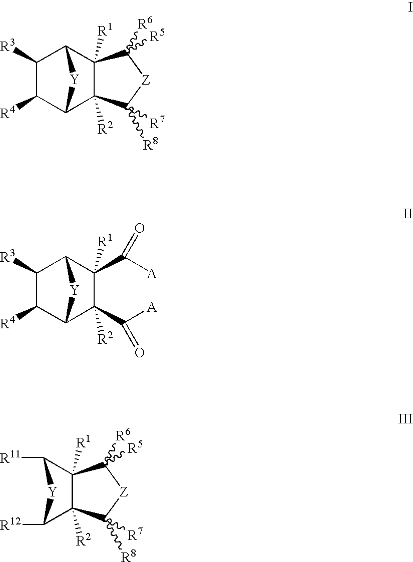
wherein
R¹ and R² are independently H or CH₃;
R³ and R⁴ are independently H, C₁-C₆ alkyl, aryl, or ara (C₁-C₁₀)alkyl; or together R³ and R⁴ form a bond (i.e., to form a cyclohexenyl ring);
R⁵, R⁶, R⁷, and R⁸ are independently H or OH, or R⁵ and R⁶, or R⁷ and R⁸ together with the carbon to which they are attached, form C═O;
R¹¹ and R¹² are independently H, C₁-C₁₀ alkyl, aryl, or ara(C₁-C₁₀)alkyl;
Y is O, N, or S;
A is OH or OR¹⁰, wherein R¹⁰ is C₁-C₆ alkyl;
Z is O, S, SR¹⁴, N—R⁹, CH₂OR², CHQ, or an amino acid;
R¹⁴ is C₁-C₁₀ alkyl, aryl, or ara(C₁-C₁₀)alkyl;
R⁹ is C₁-C₁₀ alkyl, H, OH, or Q;
R¹² is H or C₁-C₁₀ alkyl, aryl, or ara(C₁-C₁₀)alkyl;
wherein when Z is an amino acid, the α-amino group is a ring atom in a five-membered ring of formula I or III;
wherein Q is H or a moiety having the formula 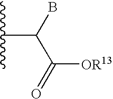
wherein R¹³ is C₁-C₁₀ alkyl or H; and B is (CH₂)ₙW, CH═CH—W, or CH₂OW, wherein W is an ionisable residue; or
a pharmaceutically acceptable salt thereof,
to the patient, wherein the patient has been diagnosed with lymphoma.

68. A method of treating cancer in a human patient, comprising administering to a human subject in need thereof a therapeutically effective regimen, the regimen comprising administering a compound of formula I, II or III 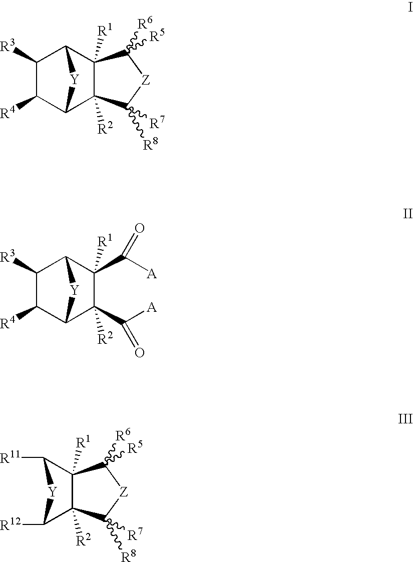
wherein
R¹ and R² are independently H or CH₃;
R³ and R⁴ are independently H, C₁-C₆ alkyl, aryl, or ara (C₁-C₁₀)alkyl; or together R³ and R⁴ form a bond (i.e., to form a cyclohexenyl ring);
R⁵, R⁶, R⁷, and R⁸ are independently H or OH, or R⁵ and R⁶, or R⁷ and R⁸ together with the carbon to which they are attached, form C═O;
R¹¹ and R¹² are independently H, C₁-C₁₀ alkyl, aryl, or ara(C₁-C₁₀)alkyl;
Y is O, N, or S;
A is OH or OR¹⁰, wherein R¹⁰ is C₁-C₆ alkyl
Z is O, S, SR¹⁴, N—R⁹, CH₂OR¹², CHQ, or an amino acid;
R¹⁴ is C₁-C₁₀ alkyl, aryl, or ara(C₁-C₁₀)alkyl;
R⁹ is C₁-C₁₀ alkyl, H, OH, or Q;
R¹² is H or C₁-C₁₀ alkyl, aryl, or ara(C₁-C₁₀)alkyl;
wherein when Z is an amino acid, the α-amino group is a ring atom in a five-membered ring of formula I or III;
wherein Q is H or a moiety having the formula 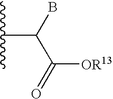
wherein R¹³ is C₁-C₁₀ alkyl or H; and B is (CH₂)ₙW, CH═CH—W, or CH₂OW, wherein W is an ionisable residue; or
a pharmaceutically acceptable salt thereof,
to the patient, wherein the compound is administered to patient at a dose lower than the maximum tolerated dose over a period of 1 to 12 months.

69. A method of treating cancer in a human patient, comprising administering to a human subject in need thereof a therapeutically effective regimen, the regimen comprising administering a compound of formula I, II or III 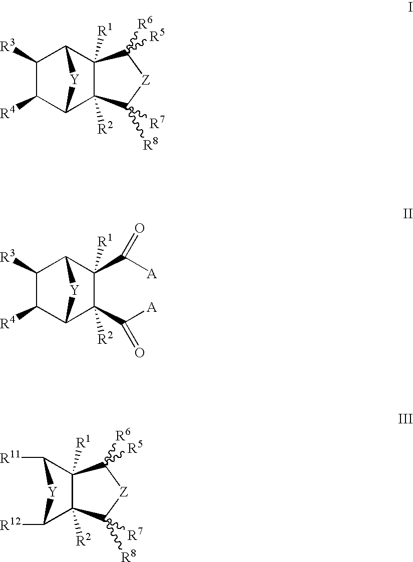
wherein
R¹ and R² are independently H or CH₃;
R³ and R⁴ are independently H, C₁-C₆ alkyl, aryl, or ara (C₁-C₁₀)alkyl; or together R³ and R⁴ form a bond (i.e., to form a cyclohexenyl ring);
R⁵, R⁶, R⁷, and R⁸ are independently H or OH, or R⁵ and R⁶, or R⁷ and R⁸ together with the carbon to which they are attached, form C═O;
R¹¹ and R¹² are independently H, C₁-C₁₀ alkyl, aryl, or ara(C₁-C₁₀)alkyl;
Y is O, N, or S;
A is OH or OR¹⁰, wherein R¹⁰ is C₁-C₆ alkyl;
Z is O, S, SR¹⁴, N—R⁹, CH₂OR¹², CHQ, or an amino acid;
R¹⁴ is C₁-C₁₀ alkyl, aryl, or ara(C₁-C₁₀)alkyl;
R⁹ is C₁-C₁₀ alkyl, H, OH, or Q;
R¹² is H or C₁-C₁₀ alkyl, aryl, or ara(C₁-C₁₀)alkyl;
wherein when Z is an amino acid, the α-amino group is a ring atom in a five-membered ring of formula I or III;
wherein Q is H or a moiety having the formula 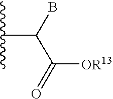
wherein R¹³ is C₁-C₁₀ alkyl or H; and B is (CH₂)ₙW, CH═CH—W, or CH₂OW, wherein W is an ionisable residue; or
a pharmaceutically acceptable salt thereof,
to the patient, wherein the compound is administered to patient at a dose lower than the human equivalent of the no observed adverse effect level (NOAEL) over a period of 1 to 12 months.
